(19) United States
(12) Patent Application Publication
(10) Pub. No.: US 2020/0239575 A1
(43) Pub. Date: Jul. 30, 2020

(54) A FUSION PROTEIN FOR TARGETED THERAPY OF AUTOIMMUNE DISEASE

(71) Applicant: University of Utah Research Foundation, Salt Lake City, UT (US)

(72) Inventors: Mingnan Chen, Salt Lake City, UT (US); Peng Zhao, Salt Lake City, UT (US); Peng Wang, Salt Lake City, UT (US)

(21) Appl. No.: 16/652,629

(22) PCT Filed: Oct. 5, 2018
(86) PCT No.: PCT/US2018/054645

§ 371 Date: Mar. 31, 2020

Related U.S. Application Data
(60) Provisional application No. 62/568,849, filed on Oct. 6, 2017.
(60) Provisional application No. 62/568,880, filed on Oct. 6, 2017.

(51) Int. Cl. C07K 16/28; C07K 14/21; C07K 14/415

(52) U.S. Cl. CPC C07K 16/2818; A61K 2039/505; C07K 14/415; C07K 14/21

(57) ABSTRACT

Disclosed herein, are fusion proteins comprising a targeting moiety, a plasma protein binding domain, and a toxin or biological variant thereof. Also described herein, are methods of administering the fusion proteins to patients with an autoimmune disease.

### CROSS REFERENCE TO RELATED APPLICATIONS

This application claims the benefit of U.S. Provisional Applications Nos. 62/568,949, and 62/568,880 both filed Oct. 6, 2017. The content of these earlier filed applications is hereby incorporated by reference herein in their entirety.

### STATEMENT REGARDING FEDERALLY SPONSORED RESEARCH

This invention was made with government support under grant no. R21EB024083 awared by the National Institutes of Health. The government has certain rights in the invention.

### INCORPORATION OF THE SEQUENCE LISTING

The present application contains a sequence listing that is submitted via EFS-Web concurrent with the filing of this application, containing the file name “21101_0345P1_SL.txt” which is 12,288 bytes in size, created on Sep. 28, 2018, and is herein incorporated by reference in its entirety.

### BACKGROUND

Autoimmune diseases (ADs) affect 50 million Americans and 12.5% people worldwide. For the vast majority of ADs, there is no cure. Rather, symptoms of AD are managed with a tremendous burden of time, effort, and cost.

Type I diabetes (T1D) currently affects the quality of life of 1.25 million Americans. While diet control and insulin administration have been widely used to cope with symptoms of T1D, no treatment is currently available to resolve its the root cause—the autoimmune destruction of insulin-producing pancreatic β-cells (Bluestone, J. A., K. Herold, and G. Eisenbarth, Genetics, pathogenesis and clinical interventions in type[thinsp]1diabetes. Nature, 2010. 464(7293): p. 1293-1300; Atkinson, M. A., G. S. Eisenbarth, and A. W. Michels, Type 1 diabetes. Lancet, 2014. 383(9911): p. 69-82; van Belle, T. L., K. T. Coppieters, and M. G. von Herrath, Type 1 diabetes: etiology, immunology, and therapeutic strategies. Physiol Rev, 2011. 91(1): p. 79-118; Nathan, D. M., Diabetes: Advances in Diagnosis and Treatment. JAMA, 2015. 314(10): p. 1052-62; and Shoda, L. K., et al., A comprehensive review of interventions in the NOD mouse and implications for translation. Immunity, 2005. 23(2): p. 115-26). Depletion of all B or T lymphocytes have been tried as a strategy to stop the destruction (van Belle, T. L., K. T. Coppieters, and M. G. von Herrath, Type 1 diabetes: etiology, immunology, and therapeutic strategies. Physiol Rev, 2011. 91(1): p. 79-118). However, such blunt depletions severely undermine the immune system and hence increase the risks of infections (Elsegeiny, W., et al., Anti-CD20 antibody therapy and susceptibility to Pneumocystis pneumonia. Infect Immun, 2015. 83(5): p. 2043-52) and cancer, not to mention that these depletions are, in principle, unnecessary. Therefore, alternative approaches are needed for treating autoimmune disorders, including T1D.

### SUMMARY

Disclosed herein, are fusion proteins comprising a targeting moiety, a plasma protein binding domain, and a toxin or biological variant thereof.

Disclosed herein are methods of inducing apoptosis, the method comprising: contacting a cell with a composition comprising a fusion protein, wherein the fusion protein comprises a single chain variable fragment (scFv) of an anti-PD-1-antibody, a plasma protein binding domain and a toxin or a biological variant thereof wherein the contacting of the cells with the composition induces apoptosis.

Disclosed herein are methods of preventing or halting cell death of one or more islet cells, the method comprising: contacting one or more lymphocytes with a composition comprising a fusion protein, wherein the fusion protein comprises a single chain variable fragment (scFv) of an anti-PD-1 antibody, a plasma protein binding domain and a toxin or biological variant thereof, wherein the contacting of the one or more lymphoctyes with the composition prevents or halts cell death.

Disclosed herein, are kits comprising a targeting moiety, a plasma protein binding domain, and a toxin or biological variant thereof.

Other features and advantages of the present compositions and methods are illustrated in the description below, the drawings, and the claims.

### BRIEF DESCRIPTION OF THE DRAWINGS

FIG. 1 shows the mechanism of by which a PD-1-positive cell is depleted by a PD-1-targeted toxin, αPD-1-ABD-PE.

FIGS. 2A-D show that αPD-1-ABD-PE is functional in vitro and in vivo. FIG. 2A shows the sequence design of αPD-1-ABD-PE. FIG. 2B The binding of mouse albumin (lane 1, arrow) and αPD-1-ABD-PE (lane 2). A mixture of the albumin and αPD-1-ABD-PE was loaded into the lane 3. The arrow in lane 3 is pointed to the albumin/αPD-1-ABD-PE complex. FIG. 2C shows the cytotoxicity of αPD-1-ABD-PE and its untargeted control, PE, to PD-1-positive cells (EL4) and PD-1-negative cells (B16). FIG. 2D shows that NOD mice treated with αPD-1-ABD-PE resist the diabetes exacerbation effect of αPD-1.

FIG. 3 shows a plan for a TID reversal study.

FIGS. 4A-D show the functionality characterization of αPD-1-ABD-PE. FIG. 4A shows the sequence design of αPD-1-ABD-PE. FIG. 4B is a bar graph showing that αPD-1-ABD-PE has a 11.7-fold greater binding and uptake by EL4 cells (PD-1-positive) than by B16 cells (PD-1-negative). FIG. 4C shows that αPD-1-ABD-PE is more toxic to EL4 cells than to B16 cells by 1000 times (IC₅₀s: EL4, 1.1 nM; B16, 1.9 μM) and that αPD-1-ABD-PE is more toxic to EL4 cells than PE by 1000 times (IC₅₀ of PE to EL4, 1.6 αM). FIG. 4D shows that the elimination half-life of αPD-1-ABD-PE is 58 times of that of αPD-1-PE (n=3).

FIGS. 5A-C show the specificity and the immune compatibility of PD-1-targeted depletion. FIG. 5A shows that αPD-1-ABD-PE reduces the ELA4 fraction of lymphocytes in mice that were transferred with EL4 cells because the toxin is able to deplete PD-1-positive cells. FIG. 5B shows mice that were treated with one dose of αPD-1-ABD-PE had comparable fractions of B220-, CD4-, and CD8-positive cells as PBS-treated mice. FIG. 5C shows that immediately (2 days) after the αPD-1-ABD-PE treatment, mice were able to mount same strength of anti-DNP antibody responses as the PBS-treated mice.

FIG. 6 shows that a single dose of αPD-1-ABD-PE (5 mg/kg) cured mice that had paralyzed hind limbs, a severe presentation of multiple sclerosis.

FIG. 7 shows that five doses of αPD-1-ABD-PE delay the onset of type 1 diabetes cyclophosphamide-induced T1D mice. The arrows indicate the time of treatments.

FIGS. 8A-G shows the configurations of constructs, amino acid sequences of the complementarity-determining regions and the results of using the same. FIG. 8A shows the sequential configurations of the functional domains, αPD-1, ABD, and PE, in αPD-1, ABD-PE, αPD-1-PE, and αPD-1-ABD-PE. The linker, (GGGGS)₃ (SEQ ID NO: 1), is shown as an orange box. FIG. 8B shows the amino acid sequences of αPD-1 V_{H} and V_{L} with their framework regions (FRs) and CDRs highlighted with red text. Two mutations were introduced in the V_{H} and V_{L}, respectively. The two mutations are underlined, V_{H}: R45C; V_{L}: G104C. SEQ ID NO: 20 represents the heavy chain sequence and SEQ ID NO: 21 represents the light chain sequence. FIG. 8C shows the mean fluorescence intensity (MFI) of PD-1⁺ and PD-1⁻ primary T cells after the cells were incubated with Alexa Fluor 647-labeled αPD-1-ABD-PE or Alexa Fluor 647-labeled ABD-PE at 4° C. for 30 minutes. The cells were collected from C57BL/6 mice. The MFI means and their standard deviations (SDs) are indicated (N=6). The MFI was obtained by flow cytometry. The experiment was repeated twice and the data of one repeat is shown here. FIG. 8D shows that the MFI of PD-1⁺ and PD-1⁻ primary B cells after the cells were incubated with the labeled αPD-1-ABD-PE or the labeled ABD-PE at 4° C. for 30 minutes. The cells were collected from C57BL/6 mice. The MFI means and their SDs are indicated (N=6). The experiment was repeated twice and the data of one repeat is shown here. FIG. 8E shows that the MFI of PD-1⁺ and PD-1⁻ primary T cells after the cells were incubated with the labeled αPD-1-ABD-PE or the labeled ABD-PE at 37° C. for 30 minutes. The cells were collected from C57BL/6 mice. The MFI means and their SDs are indicated (N=6). The experiment was repeated twice and the data of one repeat is shown here. FIG. 8F show that the MFI of PD-1⁺ and PD-1⁻ primary B cells after the cells were incubated with the labeled αPD-1-ABD-PE or the labeled ABD-PE at 37° C. for 30 minutes. The cells were collected from C57BL/6 mice. The MFI means and their SDs are indicated (N=6). The experiment was repeated twice and the data of one repeat is shown here. FIG. 8G shows that the MFI of EL4 cells after the cells were incubated with the labeled αPD-1-ABD-PE under the conditions noted in the figure. The MFI means and their SDs are indicated (N=6). The experiment was repeated twice and the data of one repeat is shown here.

FIGS. 9A-B show a photos of an agarose gel and SDS-PAGE gel. FIG. 9A is a photo of an agarose gel that contains the coding genes of αPD-1 (lane 1), ABD-PE (lane 2), αPD-1-PE (lane 3), and αPD-1-ABD-PE (lane 4). The lower bands of each lane were the coding genes after they were cleaved from the pET25b(+) vector (upper bands in each lane) by XbaI and BamHI. FIG. 9B is an SDS-PAGE gel photo of purified αPD-1, ABD-PE, αPD-1-PE, and αPD-1-ABD-PE (5 μg of each sample). Lane 1: αPD-1, lane 2: ABD-PE, lane 3: αPD-1-PE, lane 4: αPD-1-ABD-PE.

FIGS. 10A-I show the results of experiments using EL4 and B16 cells. Two representative histograms resulting from flow cytometry analyses of EL4 (A) and B16 cells (B). These cells were stained with either APC-labeled αPD-1 (full IgG, 100 nM, Red), APC-RatIgG2a isotype control (100 nM, Blue), or nothing (Green). Results reflected from these histograms suggest that the vast majority of EL4 cells express PD-1 on their surface, while almost no B16 cell express PD-1 on their surface. FIG. 10C shows MFI of EL4 cells (PD-1⁺) and B16 cells (PD-1⁻) after the cells were incubated with Alexa Fluor 647-labeled αPD-1-ABD-PE or Alexa Fluor 647-labeled ABD-PE at 4° C. for 30 minutes. The cells were collected from NOD mice. The MFI means and their SDs are indicated (N=3). FIG. 10D shows a dose-response binding study of αPD-1-ABD-PE and αPD-1 (full IgG) with EL4 cells. 1 million EL4 cells was used for the assay at 4° C. for 30 minutes. The binding affinity (Kd) were derived from sigmoidal dose-response analysis of the curves (N=6). FIG. 10E shows MFI of EL4 and B16 cells after the cells were incubated with the labeled αPD-1-ABD-PE or the labeled ABD-PE at 37° C. for 30 minutes. The MFI means and their SDs are indicated (N=3). FIG. 10F shows MFI of PD-1⁺ and PD-1⁻ primary T cells after the cells were incubated with the labeled αPD-1-ABD-PE or the labeled ABD-PE at 4° C. for 30 minutes. The cells were collected from NOD mice. The MFI means and their SDs are indicated (N=6). FIG. 10G shows MFI of PD-1⁺ and PD-1⁻ primary B cells after the cells were incubated with the labeled αPD-1-ABD-PE or the labeled ABD-PE at 4° C. for 30 minutes. The cells were collected from NOD mice. The MFI means and their SDs are indicated (N=6). FIG. 10H MFI of PD-1⁺ and PD-1⁻ primary T cells after the cells were incubated with the labeled αPD-1-ABD-PE or the labeled ABD-PE at 37° C. for 30 minutes. The cells were collected from NOD mice. The MFI means and their SDs are indicated (N=6). FIG. 10I shows MFI of PD-1⁺ and PD-1⁻ primary B cells after the cells were incubated with the labeled αPD-1-ABD-PE or the labeled ABD-PE at 37° C. for 30 minutes. The cells were collected from NOD mice. The MFI means and their SDs are indicated (N=6).

FIGS. 11A-D shows the MFI of PD-1⁺ primary T cells (A, C) and PD-1⁺ primary B cells (B, D) after the cells were incubated with Alexa Fluor 647-labeled αPD-1-ABD-PE under the conditions noted in the figure. The MFI means and their SDs are indicated (N=6). Cells of “a” and “b” were collected from C57BL/6 mice; cells of “c” and “d” were collected from NOD mice.

FIGS. 12A-D show the relative viability of PD-1⁺ and PD-1⁻ primary T and B cells. FIG. 12A shows the relative viability of PD-1⁺ and PD-1⁻ primary T cells after they were incubated with αPD-1-ABD-PE or a control mixture of αPD-1 and ABD-PE for 72 hours. The mean viabilities and their SDs at different concentrations of αPD-1-ABD-PE and the control mixture are shown. The viability data of PD-1⁺ primary T cells after the αPD-1-ABD-PE treatment were fitted to a Sigmoidal dose-response model (N=6) and the IC₅₀ was obtained through the fitting. The cells were collected from C57BL/6 mice. The experiment was repeated twice and the data of one repeat is shown here. FIG. 12B shows the relative viability of PD-1⁺ and PD-1⁻ primary B cells after they were incubated with αPD-1-ABD-PE or a control mixture of αPD-1 and ABD-PE for 72 hours. The mean viabilities and their SDs at different concentrations of αPD-1-ABD-PE and the control mixture were shown (N=6). The cells were collected from C57BL/6 mice. The experiment was repeated twice and the data of one repeat is shown here. FIG. 12C shows the relative viability of wildtype EL4 and PD-1⁻ EL4 cells after they were incubated with αPD-1-ABD-PE or a control mixture of αPD-1 and ABD-PE for 72 hours. The mean viabilities and their SDs at different concentrations of αPD-1-ABD-PE and the control mixture were shown and fitted to a Sigmoidal dose-response model (N=6). The experiment was repeated twice and the data of one repeat is shown here. FIG. 10D show the fractions of transferred EL4 cells among lymphocytes. These lymphocytes were collected from mice at 72 hours after these mice were treated with αPD-1-ABD-PE, a control mixture of αPD-1 and ABD-PE, or PBS. The mean faction values and their SDs are indicated (N=3). The experiment was repeated twice and the data of one repeat is shown here.

FIGS. 13A-C show the relative viability of PD-1⁺ and PD-1⁻ primary T and B cells, and representative scatter plots. The relative viability of PD-1⁺ and PD-1⁻ primary T cells (A) and B cells (B) after they were incubated with αPD-1-ABD-PE or a control mixture of αPD-1 and ABD-PE for 72 hours. The mean viabilities and their SDs at different concentrations of αPD-1-ABD-PE and the control mixture were shown. The viability data of PD-1⁺ primary cells after the αPD-1-ABD-PE treatment were fitted to a sigmoidal dose-response model (N=6) and the IC₅₀ was obtained through the fitting. The cells were collected from NOD mice. FIG. 13C are representative scatterplots showing the PD-1 expression on EL4 and PD-1⁻ EL4 cells. mCherry is the transfection marker. Left: EL4 cells stained with isotype control; middle: EL4 cells stained with αPD-1 (Clone: RMP1-30); right: PD-1-EL4 cell stained with αPD-1.

FIGS. 14A-D shows photos of PAGE gels, pharmokinetic results. FIG. 14A is a photo of native PAGE gel that demonstrates the association between αPD-1-ABD-PE and MSA. Lane 1: αPD-1-ABD-PE, lane 2: MSA, lane 3: a mixture αPD-1-ABD-PE and MSA at the 1-to-1 ratio, lane 4: αPD-1-PE, lane 5: a mixture αPD-1-PE and MSA at the 1-to-1 ratio. FIG. 14B is a photo of native PAGE gel that demonstrates the association between αPD-1-ABD-PE and HSA. Lane 1: αPD-1-ABD-PE, lane 2: HSA, lane 3: a mixture αPD-1-ABD-PE and HSA at the 1-to-1 ratio, lane 4: αPD-1-PE, lane 5: a mixture αPD-1-PE and HSA at the 1-to-1 ratio. FIG. 14C shows the plasma concentration versus time profiles of αPD-1-ABD-PE and αPD-1-PE after the two protein were intraperitoneally injected into mice at the same dose 5 nmol per mouse (N=3). The PK data was analyzed using non-compartmental model. Each dot represents a plasma concentration value at a given time points. FIG. 14D is a table to summarize key PK parameters derived from non-compartmental analysis.

FIGS. 15A-I shows results from NOD mice and cell fractions. FIG. 15A shows diabetes-free survival of NOD mice that were treated with αPD-1-ABD-PE, a control mixture of αPD-1 and ABD-PE, or PBS weekly since there mice were 12 weeks old (N=5). FIG. 15B shows diabetes-free survival of NOD mice that were treated five times with αPD-1-ABD-PE, a control mixture of αPD-1 and ABD-PE, or PBS (N=5). The arrows indicate the five dosing dates of the treatments. Before these treatments, these mice were treated with CP at day 0. FIG. 15C shows The fraction of PD-1⁺ cells among collected pancreatic cells from 18-week old NOD mice after these mice were treated with one dose of αPD-1-ABD-PE, a control mixture of αPD-1 and ABD-PE, or PBS. Each dot represents the fraction value of a single mouse. The fraction means and their SDs are indicated. (N=6; unpaired t-test). FIG. 15D The fraction of PD-1⁺ CD4 T cells among the pancreatic cells described in (c). Each dot represents the fraction result of a single mouse. The fraction means and their SDs are indicated. (N=6; unpaired t-test). FIG. 15E shows the fraction of PD-1⁺ CD8 T cells among the pancreatic cells described in (c). Each dot represents the fraction result of a single mouse. The fraction means and their SDs are indicated. (N=6; unpaired t-test). FIG. 15F shows the fraction of CD4 T cells among the pancreatic cells described in (c). Each dot represents the fraction result of a single mouse. The fraction means and their SDs are indicated. (N=6; unpaired t-test). FIG. 15G shows the fraction of CD8 T cells among the pancreatic cells described in (c). Each dot represents the fraction result of a single mouse. The fraction means and their SDs are indicated. (N=6; unpaired t-test). FIG. 15H shows the fraction of B cells among the pancreatic cells described in (c). Each dot represents the fraction result of a single mouse. The fraction means and their SDs are indicated. (N=6; unpaired t-test). FIG. 15I shows the diabetes-free survival of NOD mice that were first treated with αPD-1-ABD-PE, a control mixture of αPD-1 and ABD-PE, or PBS, and then with αPD-1 (full IgG). (N=5) The survival of αPD-1-ABD-PE treated mice is significantly different to PBS and the control mixture treated mice (P=0.0494 and P=0.0018, respectively).

FIGS. 16A-C show gating strategies used to quantify PD-1+CD4 T cells, Tregs, MOG₃₈₋₄₉-specific CD4 T cells (A), PD-1+CD8 T cells (B), and PD-1+B220 cells (C) by flowcytometry. Dead cells were first stained by 3 μM DAPI for 10 min, and then washed 3 times by a centrifugation (300 g for 5 minutes) to remove free DAPI. Then, cells were stained for markers indicated in the figures by antibodies.

FIGS. 17A-G show scatter plots. FIG. 17A shows the MFI (PD-1 expression) of PD-1⁺ cells in pancreases of 18-week old NOD mice after these mice were treated with one dose of αPD-1-ABD-PE, a control mixture of αPD-1 and ABD-PE, or PBS. Each dot represents the MFI of the cells from a single mouse. The MFI means and their SDs are indicated. (N=6; unpaired t-test). FIGS. 17B-C show Representative scatter plots for Tregs (B) and B cells (C) in a pancreas that was collected from the NOD mice treated with PBS. The cells were stained with αPD-1 to show PD-1⁺ populations. There was no PD-1⁺ Treg or PD-1⁺ B cell population in the pancrease. The plots represent 6 mice that were retreated with PBS. Results for the mice treated with αPD-1-ABD-PE and the control mixture were the same. FIGS. 17D-I show representative scatter plots for T cells (d, f, h) and B cells (e, g, i) in blood (d, e), spleens (f, g), and lymph nodes (h, i) that was collected from NOD mice treated with PBS. The cells were stained with αPD-1 to reveal PD-1⁺ populations. There was no PD-1⁺ T or PD-1⁺ B cell population in these samples. The plots represent 6 mice that were retreated with PBS. Results for the mice treated with αPD-1-ABD-PE and the control mixture were the same.

FIGS. 18A-I shows results from an EAE mouse model and cell fractions. FIG. 18A shows clinical score changes of the mice with EAE that were treated with one dose of αPD-1-ABD-PE, a control mixture of αPD-1 and ABD-PE, or PBS. The shown data are mean scores and their standard deviations at each observation time point after the induction of EAE. (N=6). The X-axis indicates the number of days after EAE induction. FIG. 18B shows clinical score changes of the mice described in (a). The shown data are mean scores and their standard deviations at each observation time point after the treatments. The mean clinical score of the αPD-1-ABD-PE group was different from the score of the PBS group since day 3 post treatment (*P<0.0001; unpaired t-test). The X-axis indicates the number of days since an individual mouse received treatments. The experiment described in “a” and “b” was repeated trice and the data of one repeat is shown here. FIG. 18C shows the fraction of PD-1⁺ cells among the collected mononuclear cells from the CNS of the mice that were treated with one dose of αPD-1-ABD-PE, a control mixture of αPD-1 and ABD-PE, or PBS. Each dot represents the fraction result of a single mouse. The fraction means and their SDs are indicated. (N=6; unpaired t-test). FIG. 18D shows the fraction of PD-1⁺ CD4 T cells among the mononuclear cells described in (c). Each dot represents the fraction result of a single mouse. The fraction means and their SDs are indicated (N=6; unpaired t-test). FIG. 18E shows the fraction of PD-1⁺ CD8 T cells among the mononuclear cells described in (c). Each dot represents the fraction result of a single mouse. The fraction means and their SDs are indicated (N=6; unpaired t-test). FIG. 18F shows the fraction of CD4 T cells among the mononuclear cells described in (c). Each dot represents the fraction result of a single mouse. The fraction means and their SDs are indicated (N=6; unpaired t-test). FIG. 18G shows the fraction of CD8 T cells among the mononuclear cells described in (c). Each dot represents the fraction result of a single mouse. The fraction means and their SDs are indicated (N=6; unpaired t-test). FIG. 18H shows the fraction of B cells among the mononuclear cells described in (c). Each dot represents the fraction result of a single mouse. The fraction means and their SDs are indicated (N=6; unpaired t-test). FIG. 18I shows the ratios between Tregs and the MOG-specific CD4 T cells in the mononuclear cells described in (c). Each dot represents the ratio result of a single mouse. The fraction means and their SDs are indicated (N=6; unpaired t-test).

FIGS. 19A-L show results in EAE mice. FIG. 19A shows the EAE score changes of mice that were treated with one dose of αPD-1-ABD-PE, a control mixture of αPD-1 and ABD-PE, or PBS. The shown data are mean EAE clinical scores and their standard deviations at each observation time point after the induction of EAE. (N=5). The X-axis represents the number of days after treatment started for an individual mouse. FIG. 19B shows the MFI (PD-1 expression) of PD-1⁺ cells in the CNS of the mice that were treated with one dose of αPD-1-ABD-PE, a control mixture of αPD-1 and ABD-PE, or PBS. Each dot represents the MFI of the cells from a single mouse. The MFI means and their SDs are indicated. (N=6; unpaired t-test). FIGS. 19C-D show representative scatter plots for Tregs (c) and B cells (d) in the CNS of mice with EAE and treated with PBS. The cells were stained with αPD-1 to show PD-1⁺ populations. There was no PD-1⁺ Treg or PD-1⁺ B cell population in the CNS. The plots represent 6 mice that were treated with PBS. Results for the mice treated with αPD-1-ABD-PE and the control mixture were the same. FIGS. 19E-F show the fraction of Tregs cells (d) and MOG₃₈₋₄₉-specific CD4 T cells (e) in the collected mononuclear cells from the CNS of the mice that were treated with one dose of αPD-1-ABD-PE, a control mixture of αPD-1 and ABD-PE, or PBS. Each dot represents the fraction result of a single mouse. The fraction means and their SDs are indicated. (N=6). FIGS. 19G-L show representative scatter plots for T cells (g, i, k) and B cells (h, j, 1) in blood (g,h), spleens (i, j), and lymph nodes (k, 1) that was collected from the mice with EAE and treated with PBS. The cells were stained with αPD-1 to reveal PD-1⁺ populations. There was no PD-1⁺ T or PD-1⁺ B cell population in these samples. The plots represent 6 mice that were reteated with PBS. Results for the mice treated with αPD-1-ABD-PE and the control mixture were the same.

FIGS. 20A-F show cell numbers, ELISA results and ELISPOT results. FIGS. 20A-B show B220+, CD4+, CD8+ cell numbers in blood (a) and spleens (b) in the C57BL/6 mice that were treated with one dose of αPD-1-ABD-PE, a control mixture of αPD-1 and ABD-PE, PBS, or CP. The cell number means and their SDs are indicated (N=6). FIG. 20C shows ELISA results of the anti-DNP humoral responses in the C57BL/6 mice that that were pre-treated with one dose of αPD-1-ABD-PE, a control mixture of αPD-1 and ABD-PE, PBS, or CP. The results were measured by OD₄₅₀ after a background OD₅₇₀ subtraction. The mean±SD of OD₄₅₀ for the serum samples at indicated dilutions were shown. The same samples were loaded into both DNP-BSA-coated and BSA-coated (control) ELISA plates, separately. The materials used to coat the plates are written in the parentheses. (N=6). FIG. 20D shows ELISA results of the anti-DNP humoral responses in the NOD mice that were treated with one dose of αPD-1-ABD-PE, a control mixture of αPD-1 and ABD-PE, PBS, or CP. The results were measured by OD₄₅₀ after a background OD₅₇₀ subtraction. The mean±SD of OD₄₅₀ for the serum samples at indicated dilutions were shown. The materials used to coat ELISA plates are written in the parentheses. (N=6). FIG. 20E shows ELISPOT results of the CTL responses in the C57BL/6 mice that were treated with one dose of αPD-1-ABD-PE, a control mixture of αPD-1 and ABD-PE, PBS, or CP. The Y-axis represents the number of IFN-γ-positive spots resulting from the 500,000 splenocytes collected from these treated mice. The means and their SDs of the spot numbers are indicated. (N=6: ns, not significant; unpaired t-test). FIG. 20F shows ELISPOT results of the CTL responses in the NOD mice that were treated with one dose of αPD-1-ABD-PE, a control mixture of αPD-1 and ABD-PE, PBS, or CP. The Y-axis represents the number of IFN-γ-positive spots resulting from the 500,000 splenocytes collected from these treated mice. The means and their SDs of the spot numbers are indicated. (N=6: ns, not significant; unpaired t-test). The studies described in this figure were repeated at least twice and the data of one repeat is shown here.

FIGS. 21A-H show cell numbers, ELISA results and ELISPOT results. FIGS. 21A-B show B220+, CD4+, CD8+ cell numbers in blood (a) and spleens (b) in the NOD mice that were treated with one dose of αPD-1-ABD-PE, a control mixture of αPD-1 and ABD-PE, PBS, or CP. The number means and their SDs are indicated (N=6). FIGS. 21C-D show the data curves in FIG. 6c are separated into four subfigures based on the treatments. The subfigures are prepared to show curve details that are hidden due the overlap of these curves. FIGS. 21G-H ELISA results of the anti-DNP humoral responses in the C57BL/6 mice (g) and the NOD mice (h) that that were pre-treated with five doses of αPD-1-ABD-PE, a control mixture of αPD-1 and ABD-PE, or PBS. The results were measured by OD₄₅₀ after a background OD₅₇₀ subtraction. The mean±SD of OD₄₅₀ for the serum samples at indicated dilutions were shown. The same samples were loaded into both DNP-BSA-coated and BSA-coated (control) ELISA plates, separately. The materials used to coat the plates are written in the parentheses. (N=5).

### DETAILED DESCRIPTION

The present disclosure can be understood more readily by reference to the following detailed description of the invention, the figures and the examples included herein.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, the term “subject” refers to the target of administration, e.g., a human. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment for an autoimmune disorder, such as, for example, prior to the administering step.

As used herein, the term “fusion protein” refers to a composition comprising a targeting moiety, a plasma protein binding domain, and a toxin or biological variant thereof.

As used herein, the term “targeting moiety” refers to the portion of the fusion protein that specifically binds a selected target. The targeting moiety can be, for example, an polysaccharide, a peptide, peptide ligand, an aptamer, an antibody or fragment thereof, a single chain variable fragment (scFv) of an antibody, or a Fab′ fragment, or a nanobody. Targeting moieties can also include other forms of an antibody as disclosed in Rissiek et al.; “Nanobodies as modulators of inflammation: potential applications for acute brain injury,” Front. Cell. Neurosci., 21 Oct. 2014; and Cuesta et al.; “Multivalent antibodies: when design surpasses evolution;” Trends in Biotechnology; Vol. 28, Issue 7, pp. 355-362, July 2010. The cited references are incorporated herein by reference in their entirety. As used herein, a “targeting moiety” can be specific to a recognition molecule on the surface of a cell or a population of cells, such as, for example B cells or T cells. In an aspect of the disclosed compositions and methods, a targeting moiety can include, but is not limited to: a monoclonal antibody, a polyclonal antibody, full-length antibody, a chimeric antibody, Fab′, Fab, F(ab)₂, F(ab′)₂, a single domain antibody, Fv, a single chain Fv (scFv), a minibody, a diabody, a triabody, hybrid fragments, a phage display antibody, a ribosome display antibody, an oligonucleotide, a modified oligonucleotide, a peptide, a peptide ligand, a hormone, a growth factor, a cytokine, a saccharide or polysaccharide, and an aptamer.

As used herein, “aptamers” refer to molecules that interact with a target molecule, preferably in a specific way. Typically, aptamers are small nucleic acids ranging from 15-50 bases in length that fold into defined secondary and tertiary structures, such as stem-loops or G-quartets. Aptamers can bind small molecules and large molecules. Aptamers can bind very tightly with Kd's from the target molecule of less than 10⁻¹² M. Aptamers can bind the target molecule with a very high degree of specificity. Aptamers are known to the art and representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698. In an aspect, the aptamer can be synthetic, nonimmunogenic antibody mimics. In an aspect, the aptamer can be a DNA aptamer. In an aspect, the DNA aptamer can be anti-PD-1 aptamer.

As used herein, the term “contacting” refers to bringing a disclosed composition, compound, conjugate or fusion protein together with an intended target (such as, e.g., a cell or population of cells, a receptor, an antigen, or other biological entity) in such a manner that the disclosed composition, compound, conjugate or fusion protein can affect the activity of the intended target (e.g., receptor, transcription factor, cell, population of cells, etc.), either directly (i.e., by interacting with the target itself), or indirectly (i.e., by interacting with another molecule, co-factor, factor, or protein on which the activity of the target is dependent). In an aspect, a disclosed composition or fusion protein can be contacted with a cell or population of cells, such as, for example, one or more lymphocytes (e.g., T cells and/or B cells).

As used herein, the term “determining” can refer to measuring or ascertaining an activity or an event or a quantity or an amount or a change in expression and/or in activity level or in prevalence and/or incidence. For example, determining can refer to measuring or ascertaining the quantity or amount of apoptotic induction. Determining can also refer to measuring or ascertaining the quantity or amount of T cells, B cells, or pancreatic islet cells. Methods and techniques used to determining an activity or an event or a quantity or an amount or a change in expression and/or in activity level or in prevalence and/or incidence as used herein can refer to the steps that the skilled person would take to measure or ascertain some quantifiable value. The art is familiar with the ways to measure an activity or an event or a quantity or an amount or a change in expression and/or in activity level or in prevalence and/or incidence.

General

Type 1 diabetes (T1D) needs a more effective treatment with fewer side effects. Currently, diet control and insulin injection are used to manage T1D. However, these methods add a significant burden to T1D patients (Atkinson, M. A., G. S. Eisenbarth, and A. W. Michels, Type 1 diabetes. Lancet, 2014. 383(9911): p. 69-82). To date, T1D lacks a treatment that stops autoimmune destruction of β-cells and reverses the disease. Experimental strategies to stop the destruction fall into two categories: suppressing pro-inflammatory immunity and enhancing immune tolerance (Gomez-Tourino, I., et al., T cells in type 1 diabetes: Instructors, regulators and effectors: A comprehensive review. J Autoimmun, 2016. 66: p. 7-16). As one method to suppress pro-inflammatory immunity, T and B lymphocytes were bluntly depleted by using teplizumab (anti-CD3 antibody, aCD3) or rituximab (anti-CD20 antibody, aCD20) (Atkinson, M. A., G. S. Eisenbarth, and A. W. Michels, Type 1 diabetes. Lancet, 2014. 383(9911): p. 69-82; van Belle, T. L., K. T. Coppieters, and M. G. von Herrath, Type 1 diabetes: etiology, immunology, and therapeutic strategies. Physiol Rev, 2011. 91(1): p. 79-118; Shoda, L. K., et al., A comprehensive review of interventions in the NOD mouse and implications for translation. Immunity, 2005. 23(2): p. 115-26; and Zhou, Z., et al., Type 1 diabetes associated HLA-DQ2 and DQ8 molecules are relatively resistant to HLA-DM mediated release of invariant chain-derived CLIP peptides. European Journal of Immunology, 2016. 46(4): p. 834-84). However, the immune system takes a long time to recover from the blunt depletion, which increases the risk of infectious diseases and cancer. Additionally, T and B lymphocytes have not been abated concomitantly, which is too toxic to do. A concurrent depletion of T1D-related B and T lymphocytes, however, seems necessary to effectively stop autoimmune destruction as both types of lymphocytes contribute to diabetes (van Belle, T. L., K. T. Coppieters, and M. G. von Herrath, Type 1 diabetes: etiology, immunology, and therapeutic strategies. Physiol Rev, 2011. 91(1): p. 79-118). Given these considerations, an effective, low side effect T1D treatment should be one that depletes both T and B lymphocytes by targeting subpopulations of T and B lymphocytes that are closely related to T1D.

Described herein are compositions and methods to stop β-cell destruction by depleting a specific population of immune cells, programmed death-1(PD-1)-positive cells, the cells that express the PD-1 receptor on their surface. PD-1-positive cells are primarily activated T and B lymphocytes (Francisco, L. M., P. T. Sage, and A. H. Sharpe, The PD-1 Pathway in Tolerance and Autoimmunity. Immunological reviews, 2010. 236: p. 219-242), in which PD-1 mediates the PD-1 immune checkpoint. Blockade of the PD-1 immune checkpoint, which promotes the effector function and amplification of PD-1-positive cells, exacerbates diabetes in mice and humans Okamoto, M., et al., Fulminant type 1 diabetes mellitus with anti-programmed cell death-1 therapy. J Diabetes Investig, 2016. 7(6): p. 915-918; Hughes, J., et al., Precipitation of Autoimmune Diabetes With Anti-PD-1 Immunotherapy. Diabetes Care, 2015. 38(4): p. e55; and Ansari, M. J., et al., The programmed death-1 (PD-1) pathway regulates autoimmune diabetes in nonobese diabetic (NOD) mice. J Exp Med, 2003. 198(1): p. 63-9). The strategy, described herein, to deplete PD-1-positive cells as a treatment to halt autoimmune destruction of β-cells without incurring the side effects of the blunt depletions of B or T lymphocytes. The data disclosed herein supports the effectiveness of this PD-1-targeted depletion strategy. This depletion may also be more effective in stopping the destruction than blunt depletions as it concurrently abates both activated T and activated B lymphocytes, which is unprecedented (Atkinson, M. A., G. S. Eisenbarth, and A. W. Michels, Type 1 diabetes. Lancet, 2014. 383(9911): p. 69-82).

The PD-1-targeted depletion is distinct from previously immune suppression strategies used in T1D treatment in two aspects. First, the PD-1-targeted depletion applies to both T and B lymphocytes, which may make it more potent than the previous strategies. Second, the depletion applies to PD-1-positive cells instead of all T lymphocytes or B lymphocytes, which can be expected to affect the immune system minimally and cause much milder toxicity than the previous strategies. The low toxicity nature of the PD-1-targeted depletion can also allow using the fusion protein described herein to patients more frequently or at higher doses than the previous strategies, which favors it efficacy. From the novelty perspective, it is unprecedented to reverse autoimmunity disorders by depleting PD-1-positive cells although the PD-1 checkpoint has been linked to various autoimmune disorders (Zhang, Q. and D. A. Vignali, Co-stimulatory and Co-inhibitory Pathways in Autoimmunity. Immunity, 2016. 44(5): p. 1034-51).

Multiple sclerosis (MS) is also an autoimmune disease affecting approximately 400,000 people in the US and 2.5 million people worldwide; also, it is the second most common cause of young-adult disability. The root cause of MS is autoimmune attack on the white matter of the central nervous system (CNS). These attacks are executed by auto-reactive lymphocytes and auto-antibodies secreted by auto-reactive lymphocytes. To date, there is no cure for MS. Rather, current disease-modifying therapies (DMTs) delay the progression of the MS-caused disabilities by reducing the attacks. Newer and more effective DMTs utilize targeted suppression of certain lymphocyte populations to reduce the attacks; however, these newer DMTs did not become the first-line therapy due to their side effects such as lethal progressive multifocal leukoencephalopathy (PML). The side effects occur because these DMTs suppress lymphocyte too broadly, which consequently undermines normal adaptive immunity. Thus, to harness therapeutic benefits of the targeted suppression of lymphocytes without incurring the side effects, developing a therapeutic that targets a more focused lymphocyte population is needed. To this end, disclosed herein are compositions and methods that can be used to stop autoimmune attacks in MS by depleting programmed death-1 (PD-1)-positive cells, a small lymphocyte population that has not been targeted for MS treatment.

PD-1-positive cells are primarily activated B and T cells. According to results of human- and animal-based studies, these PD-1-positive cells play important roles in autoimmune attacks in MS. Therefore, depletion of PD-1-positive cells (PD-1-targeted depletion hereafter) may stop the autoimmune attacks. More significantly, PD-1-targeted depletion is expected to keep adaptive immunity intact because the depletion selectively affects activated lymphocytes but not naïve lymphocytes. Thus, this depletion should not damage the lymphocyte repertoire—adaptive immunity can maintain its full defense potential after this specific depletion. Data provided herein support the notion that PD-1-targeted depletion stops the autoimmune attack and preserves adaptive immunity. The results disclosed herein show that PD-1-targeted depletion cured paralysis in mice with severe experimental autoimmune encephalitis (EAE), a murine model of MS; second, mice that experienced the depletion mounted full-strength adaptive immune responses to antigens. Taken together, the compositions and methods disclosed herein can result in PD-1-targeted depletion that can stop an autoimmune attack without undermining adaptive immunity.

MS treatment requires targeted DMTs without associated severe side effects. To date, four targeted DMTs that suppress specific lymphocyte populations are approved including natalizumab, alematuzumab, daclizumab, and ocrelizumab. Some of these DMTs have improved efficacy over interferon-β, a mainstay of MS treatment. However, these targeted DMTs are plagued by their side effects, such as lethal PML (caused by natalizumab), as well as increased risks of infections and secondary autoimmune disorders (caused by alematuzumab). PML is particularly concerning because 50-60% of MS patients, who harbor John Cunningham polymavirus, are susceptible to PML. Current thinking suggests that the side effects are attributed to compromised adaptive immunity by the DMTs. Disclosed herein are compositions and method that can be used to treat MS treatment and can diminish autoimmune attacks without affecting normal adaptive immunity.

As mentioned above, PD-1-targeted depletion may stop autoimmune attack in MS. PD-1 is a receptor primarily expressed on activated B and T cells. PD-1, when engaged with its ligand, sustains the PD-1 immune checkpoint, a type of immune tolerance that prevents PD-1-positive cells from attacking self-tissues. This checkpoint, however, fails in MS; consequently, PD-1-positive cells are able to exert autoimmune attack on white matter of the CNS and cause demyelination. Because PD-1-positive cells are responsible for the autoimmune attack, the strategy disclosed herein is to stop the attack by depleting PD-1-positive cells. Support for this strategy comes from both murine and human studies: (1) PD-1-positive cells infiltrate the CNS during EAE; (2) blocking and disabling the PD-1 checkpoint, which expands PD-1-positive cells and aggravates EAE; (3) reinforcing the checkpoint, which suppresses PD-1-positive cells and protects mice from EAE; and (4) the PD-1 checkpoint influences the susceptibility and the progression of MS in humans. In addition to these evidences, the data disclosed herein show a single dose of the fusion protein described herein resulted in mice that were partially paralyzed due to EAE to regain normal gaits.

PD-1-targeted depletion may preserve adaptive immunity and avoid the aforementioned side effects. Because PD-1-targeted depletion applies to activated lymphocytes, it should leave naïve lymphocytes intact and preserve B and T cell repertoires. Thus, the depletion, distinct from natalizumab and alematuzumab that cause extensive damage to adaptive immunity, should not significantly compromise adaptive immunity or cause side effects like PMLs. Indeed, mice that went through the depletion were able to mount normal adaptive immune responses. Interestingly, lymphocytes that are activated in the context of dangerous signals, e.g. CpG, were found to be PD-1-negative, suggesting that PD-1-targeted depletion will not affect immune responses mediated by these activated lymphocytes.

PD-1-targeted depletion as a result to exposure to the compositions or fusion proteins disclosed herein is expected to be as efficacious as natalizumab and alematuzumab in stopping autoimmune attacks because the depletion applies to both B and T cells like these two drugs. The coverage is desired because both activated B and activated T cells exert autoimmune attacks. Further, it is a reasonable expectation that the depletion may have better overall clinical outcomes than existing DMTs because the depletion can be used more frequently than these DMTs should the depletion have no or mild side effects.

It is unprecedented to resolve autoimmune attacks in MS by depleting PD-1-positive cells. Compared to existing DMTs, PD-1-targeted depletion is different than currently available therapies for the following features: first, the depletion applies to both B and T cells; and second, the depletion, although applies to both B and T cells, selectively affects activated B and T cells. Because of these features, the depletion can preserve adaptive immunity while eradicating pathogenic lymphocytes in MS. Consequently, the depletion may have a broader therapeutic window and, in turn, better clinical outcomes than existing DMTs.

The design of PD-1-ABD-PE is unique for incorporating drug delivery principles. First, αPD-1 was exploited as a targeting moiety. Second, the PE was a re-engineered toxin that had reduced off-target toxicity and immunogenicity. Last, ABD is used to extend the half-life of αPD-1-ABD-PE and hence increase the access of αPD-1-ABD-PE to PD-1-positive cells.

αPD-1-ABD-PE is different compared to previously reported toxin for the PE and the ABD in it. This PE was re-engineered from a natural PE to possess low off-target toxicity and immunogenicity[14, 17, 18]. The PE was proven safe in clinical trials[16-18]. ABD would extend the half-life of αPD-1-ABD-PE because ABD binds with albumin[22]. Because of the longer half-life, αPD-1-ABD-PE will have more exposure to PD-1-positive cells, which can lead to an increased efficacy and reduce the dose.

Fusion Protein

Targeting Moiety.

In some aspects, the targeting moiety of the fusion protein can be a polysaccharide, peptide, peptide ligand, an apatmer, an antibody or fragment thereof, a single chain variable fragment (scFv) of antibody, a Fab′ fragment or biologically active variant thereof. For example, if the targeting moiety is an antibody, the antibody can be a single chain antibody (scFv) or Fab′ fragment; a human, chimeric or humanized antibody or a biologically active variant thereof; and/or can be (or can be derived from) a monoclonal or polyclonal antibody.

In some aspects, the targeting moiety of the fusion protein can be a non-naturally occurring antibody (e.g., a single chain antibody or diabody) or a biologically active variant thereof. As noted above, the variants include, without limitation, a fragment of a naturally occurring antibody (e.g., an Fab fragment), a fragment of a scFv or diabody, or a variant of a tetrameric antibody, an scFv, a diabody, or fragments thereof that differ by an addition and/or substitution of one or more amino acid residues. The antibody can also be further engineered.

In an aspect, the targeting moiety is a scFv or Fab fragment that binds (or specifically binds) to an immune checkpoint receptor. Examples of immune checkpoint receptors include but are not limited to PD-1, CTLA-4, CD28, ICOS, BTLA, lymphocyte activation gene 3 (LAG3), T cell immunoglobulin and mucin-3 (TIM3), B7-H3 (CD276), T cell ITIM domain (TIGIT), CD137 (4-1BB), OX40, CD27, CD40L, ICOS, and B- and T-lymphocyte attenuator (BTLA). In an aspect, the immune checkpoint receptor can be PD-1 or CTLA-4.

In other aspects, the fusion proteins, described herein, comprise a targeting moiety, wherein the targeting moiety can be a scFv of an anti-programmed cell death protein 1 (PD-1) antibody. In an aspect, the targeting moiety can be derived from an anti-PD-1 antibody. In an aspect, the targeting moiety can be an antibody. The antibody can be an anti-PD-1 antibody. Examples of anti-PD-1 antibodies include but are not limited to nivolumab, pembrolizumab, pidilizumab, MEDI0680, BMS-936559, clone J116, Keytruda®, Opdivo® or a biologically active variant thereof.

In an aspect, the scFv can be designed based on CDR information of an anti-PD-1 antibody. In an aspect, complementarity-determining regions (CDRs) of the heavy chain or light chain of an anti-PD-1 antibody can be used in to prepare an anti-PD-1 antibody or a fragment thereof (e.g. scFv). For example, disclosed herein are anti-PD-1 antibodies comprising one or more of CDRs including CDRs of the heavy chain: SSYRWN (SEQ ID NO: 22), YINSAGISNYNPSLKR (SEQ ID NO: 23), and SDNMGTTPFTY (SEQ ID NO: 24); or CDRs of the light chain: RSSKSLLYSDGKTYLN (SEQ ID NO: 25), WMSTRAS (SEQ ID NO: 26), and QQGLEFPT (SEQ ID NO: 27).

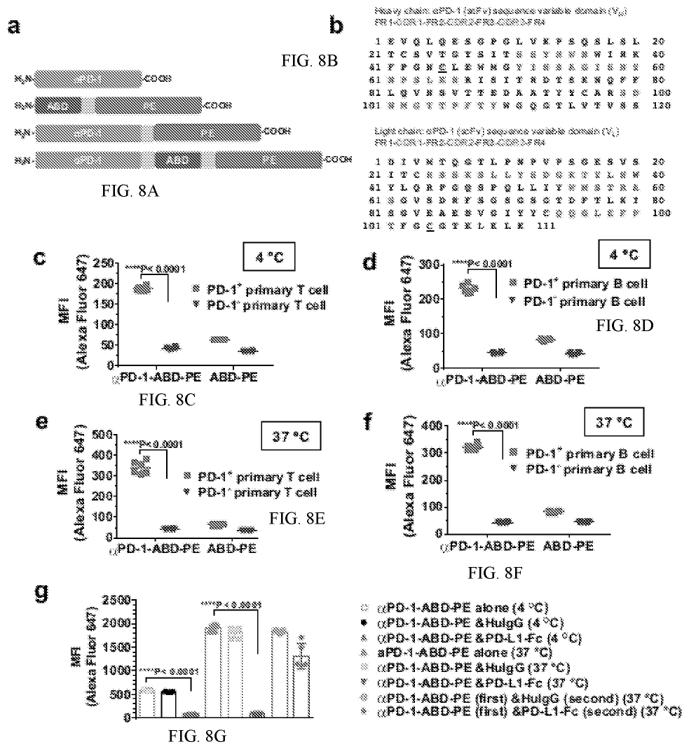

Disclosed herein are anti-PD-1 antibodies comprising mutations in the V_{H} and V_{L}, respectively of an αPD-1. For example, FIG. 8B provides an example of two mutations (underlined) wherein the mutations are V_{H}: R45C; V_{L}: G104C. In an aspect, disclosed herein can be an antibody or antigen-binding portion thereof, comprising: a heavy chain sequence and a light chain sequence, wherein the heavy chain sequence comprises SEQ ID NO: 20 and wherein the light chain sequence comprises SEQ ID NO: 21. In an aspect, the heavy and light chain sequences can exhibit a certain degree of identity or homology to the SEQ ID NOs: 20 or 21. The degree of identity can vary and be determined by methods known to one of ordinary skill in the art. The terms “homology” and “identity” each refer to sequence similarity between two polypeptide sequences. Homology and identity can each be determined by comparing a position in each sequence which can be aligned for purposes of comparison. When a position in the compared sequence is occupied by the same amino acid residue, then the polypeptides can be referred to as identical at that position; when the equivalent site is occupied by the same amino acid (e.g., identical) or a similar amino acid (e.g., similar in steric and/or electronic nature), then the molecules can be referred to as homologous at that position. A percentage of homology or identity between sequences is a function of the number of matching or homologous positions shared by the sequences. The heavy and light chain sequences of an anti-PD-1 antibody comprising one or more mutations V_{H} and V_{L}, respectively of an αPD-1 as described herein can have at least or about 25%, 50%, 65%, 75%, 80%, 85%, 90%, 95%, 96%, 97%, 98%, or 99% identity or homology to SEQ ID NOs: 20 and/or 21.

In an aspect, one or more of the heavy or light chain CDR sequences can comprise at least one substitution or at least one amino acid substitution compared to the parent heavy or light chain sequence (e.g., SEQ ID Nos: 20 or 21). In an aspect, one or more of the heavy or light chain CDR sequences can comprise at least one substitution or at least one amino acid substitution compared to the parent CDR (e.g., SEQ ID Nos: 22, 23, 24, 25, 26 or 27).

In some aspects, the CDRs disclosed herein can also include variants. Generally, the amino acid identity between individual variant CDRs can be at least 90%, 91%, 92%, 93%, 94%, 95%, 96%, 97%, 98%, 99% or 100%. Thus, a “variant CDR” can be one with the specified identity to the parent CDR as disclosed herein, and shares biological function, including, but not limited to, 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR.

Disclosed herein are anti-PD-1 antibodies comprising mutations in the V_{H} and V_{L}, respectively of an αPD-1. For example, two mutations can be V_{H}: R45C; V_{L}: G104C. In an aspect, disclosed herein is an antibody or antigen-binding portion thereof, comprising: a heavy chain sequence and a light chain sequence, wherein the heavy chain sequence comprises SEQ ID NO: 20 and wherein the light chain sequence comprises SEQ ID NO: 21, and wherein the antibody comprises one or more of CDRs selected from the group of SSYRWN (SEQ ID NO: 22), YINSAGISNYNPSLKR (SEQ ID NO: 23), SDNMGTTPFTY (SEQ ID NO: 24), RSSKSLLYSDGKTYLN (SEQ ID NO: 25), WMSTRAS (SEQ ID NO: 26), and QQGLEFPT (SEQ ID NO: 27).

Disclosed herein are antibodies or antigen-binding portion thereof, comprising: a heavy chain sequence and a light chain sequence, wherein the heavy chain sequence comprises SEQ ID NO: 20 and wherein the light chain sequence comprises SEQ ID NO: 21, and wherein the antibody comprises one or more of CDRs selected from the group of SSYRWN (SEQ ID NO: 22), YINSAGISNYNPSLKR (SEQ ID NO: 23), SDNMGTTPFTY (SEQ ID NO: 24), RSSKSLLYSDGKTYLN (SEQ ID NO: 25), WMSTRAS (SEQ ID NO: 26), and QQGLEFPT (SEQ ID NO: 27).

Disclosed herein are antibodies or antigen-binding portion thereof, comprising: a heavy chain sequence and a light chain sequence, wherein the heavy chain sequence consists of SEQ ID NO: 20 and wherein the light chain sequence consists of SEQ ID NO: 21.

Disclosed herein are antibodies or antigen-binding portion thereof, comprising: a heavy chain sequence and a light chain sequence, wherein the heavy chain sequence consists of SEQ ID NO: 20 and wherein the light chain sequence consists of SEQ ID NO: 21, and wherein the antibody comprises one or more of CDRs selected from the group of SSYRWN (SEQ ID NO: 22), YINSAGISNYNPSLKR (SEQ ID NO: 23), SDNMGTTPFTY (SEQ ID NO: 24), RSSKSLLYSDGKTYLN (SEQ ID NO: 25), WMSTRAS (SEQ ID NO: 26), and QQGLEFPT (SEQ ID NO: 27).

Disclosed herein are antibodies or antigen-binding portion thereof, comprising: a heavy chain sequence and a light chain sequence, wherein the heavy chain sequence consists of SEQ ID NO: 20 and wherein the light chain sequence consists of SEQ ID NO: 21, and wherein the antibody comprises one or more of CDRs selected from the group of SSYRWN (SEQ ID NO: 22), YINSAGISNYNPSLKR (SEQ ID NO: 23), SDNMGTTPFTY (SEQ ID NO: 24), RSSKSLLYSDGKTYLN (SEQ ID NO: 25), WMSTRAS (SEQ ID NO: 26), and QQGLEFPT (SEQ ID NO: 27).

As described herein, SEQ ID NO: 20 is an example of a heavy chain sequence and SEQ ID NO: 21 is an example of a light chain sequence

In an aspect, the scFv can be from an anti-PD-1 antibody comprising mutations in the V_{H} and V_{L}, respectively of an αPD-1. An example of two are V_{H}: R45C; V_{L}: G104C. In an aspect, disclosed herein is an antibody or antigen-binding portion thereof, comprising: a heavy chain sequence and a light chain sequence, wherein the heavy chain sequence comprises SEQ ID NO: 20 and wherein the light chain sequence comprises SEQ ID NO: 21, and wherein the antibody comprises one or more of CDRs selected from the group of SSYRWN (SEQ ID NO: 22), YINSAGISNYNPSLKR (SEQ ID NO: 23), SDNMGTTPFTY (SEQ ID NO: 24), RSSKSLLYSDGKTYLN (SEQ ID NO: 25), WMSTRAS (SEQ ID NO: 26), and QQGLEFPT (SEQ ID NO: 27).

In an aspect, the scFv can be from an antibody or antigen-binding portion thereof, comprising: a heavy chain sequence and a light chain sequence, wherein the heavy chain sequence comprises SEQ ID NO: 20 and wherein the light chain sequence comprises SEQ ID NO: 21, and wherein the antibody comprises one or more of CDRs selected from the group of SSYRWN (SEQ ID NO: 22), YINSAGISNYNPSLKR (SEQ ID NO: 23), SDNMGTTPFTY (SEQ ID NO: 24), RSSKSLLYSDGKTYLN (SEQ ID NO: 25), WMSTRAS (SEQ ID NO: 26), and QQGLEFPT (SEQ ID NO: 27).

In an aspect, the scFv can be from antibody or antigen-binding portion thereof, comprising: a heavy chain sequence and a light chain sequence, wherein the heavy chain sequence consists of SEQ ID NO: 20 and wherein the light chain sequence consists of SEQ ID NO: 21.

In an aspect, the scFv can be from antibody or antigen-binding portion thereof, comprising: a heavy chain sequence and a light chain sequence, wherein the heavy chain sequence consists of SEQ ID NO: 20 and wherein the light chain sequence consists of SEQ ID NO: 21, and wherein the antibody comprises one or more of CDRs selected from the group of SSYRWN (SEQ ID NO: 22), YINSAGISNYNPSLKR (SEQ ID NO: 23), SDNMGTTPFTY (SEQ ID NO: 24), RSSKSLLYSDGKTYLN (SEQ ID NO: 25), WMSTRAS (SEQ ID NO: 26), and QQGLEFPT (SEQ ID NO: 27)

In an aspect, the scFv can be from antibody or antigen-binding portion thereof, comprising: a heavy chain sequence and a light chain sequence, wherein the heavy chain sequence consists of SEQ ID NO: 20 and wherein the light chain sequence consists of SEQ ID NO: 21, and wherein the antibody comprises one or more of CDRs selected from the group of SSYRWN (SEQ ID NO: 22), YINSAGISNYNPSLKR (SEQ ID NO: 23), SDNMGTTPFTY (SEQ ID NO: 24), RSSKSLLYSDGKTYLN (SEQ ID NO: 25), WMSTRAS (SEQ ID NO: 26), and QQGLEFPT (SEQ ID NO: 27) In an aspect, the scFv can be from an anti-programmed death-1 antibody. In an aspect, the scFv can be from Keytruda® or Opdivo®.

Immune checkpoint inhibitors such as the anti-cytotoxic T lymphocyte antigen-4 antibody (αCTLA-4) and the anti-programmed death-1 antibody (αPD-1) have been approved to treat advanced melanoma, lung cancer, head and neck cancer, among others (Michielin, O., et al., Gaining momentum: New options and opportunities for the treatment of advanced a. Cancer Treat Rev 2015, 41 (8), 660-70; Wolchok, J. D., et al., Nivolumab plus ipilimumab in advanced melanoma. The New England journal of medicine 2013, 369 (2), 122-33; Administration, U. S. F. a. D. pembrolizumab (KEYTRUDA). http://www.accessdata.fda.gov/drugsatfda_docs/label/2016/125514s009lbl.pdf; and Sharma, P., et al., Immune checkpoint targeting in cancer therapy: toward combination strategies with curative potential. Cell 2015, 161 (2), 205-14; and Larkin, J., et al., Combined Nivolumab and Ipilimumab or Monotherapy in Untreated Melanoma. The New England journal of medicine 2015). Some of these inhibitors have been approved by the FDA, such as Pembrolizumab and Nivolumab (Swaika, A., et al., Current state of anti-PD-L1 and anti-PD-1 agents in cancer therapy. Molecular Immunology 2015, 67 (2, Part A), 4-17). Recently, αCTLA-4 and αPD-1 were combined to further boost their efficacy (Wolchok, J. D., et al., Nivolumab plus ipilimumab in advanced melanoma. The New England journal of medicine 2013, 369 (2), 122-33; and Larkin, J., et al., Combined Nivolumab and Ipilimumab or Monotherapy in Untreated Melanoma. The New England journal of medicine 2015). However, the further improvement of the immune checkpoint therapy is hindered by its autoimmune toxicity. For example, in the above combination therapy, 55% of the combination therapy patients suffered from high-grade (grades 3-4) toxicity, and 36% of the patients had to discontinue the therapy due to the toxicity (Larkin, J., et al., Combined Nivolumab and Ipilimumab or Monotherapy in Untreated Melanoma. The New England journal of medicine 2015). In contrast to the pressing need to reduce the toxicity, the current toxicity mitigating method, non-specific immune suppression, is apparently not effective enough because one third of the treated patients had to stop the therapy even after using this method, not to mention that the method has its own side effects (e.g., immune deficiency) (Tarhini, A., Immune-mediated adverse events associated with ipilimumab ctla-4 blockade therapy: the underlying mechanisms and clinical management. Scientifica (Cairo) 2013, 2013, 857519). Previously, intra-tumor injection of the inhibitors was attempted and proven effective (Fransen, M. F., et al., Controlled local delivery of CTLA-4 blocking antibody induces CD8+ T-cell-dependent tumor eradication and decreases risk of toxic side effects. Clinical cancer research: an official journal of the American Association for Cancer Research 2013, 19 (19), 5381-9); however, this method is not practical for advanced cancer patients as it is almost impossible to inject inhibitors into metastatic tumors. Therefore, new strategies are needed to reduce the toxicity of immune checkpoint inhibitors.

Intrinsically, immune checkpoints (e.g., PD-1 and CTLA-4) protect tumors from immune elimination (Topalian, S. L., et al., Targeting the PD-1/B7-H1 (PD-L1) pathway to activate anti-tumor immunity. Current Opinion in Immunology 2012, 24 (2), 207-212; and Baksh, K., et al., Immune checkpoint protein inhibition for cancer: preclinical justification for CTLA-4 and PD-1 blockade and new combinations. Semin Oncol 2015, 42 (3), 363-77), and also prevent autoimmune toxicity in healthy tissues (Pentcheva-Hoang, T., et al., Negative regulators of T-cell activation: potential targets for therapeutic intervention in cancer, autoimmune disease, and persistent infections. Immunological reviews 2009, 229 (1), 67-87). The cause of the toxicity is that the checkpoint inhibitors indiscriminately block the checkpoint in all cells that utilize the checkpoints (Pentcheva-Hoang, T., et al., Negative regulators of T-cell activation: potential targets for therapeutic intervention in cancer, autoimmune disease, and persistent infections. Immunological reviews 2009, 229 (1), 67-87; Gelao, L., et al., Immune checkpoint blockade in cancer treatment: a double-edged sword cross-targeting the host as an “innocent bystander”. Toxins 2014, 6 (3), 914-33; Nishino, M., et al., Anti-PD-1-Related Pneumonitis during Cancer Immunotherapy. New England Journal of Medicine 2015, 373 (3), 288-290; Kochupurakkal, N. M., et al., Blockade of the programmed death-1 (PD1) pathway undermines potent genetic protection from type 1 diabetes. PloS one 2014, 9 (2), e89561; Frebel, H., et al., The risks of targeting co-inhibitory pathways to modulate pathogen-directed T cell responses. Trends in immunology 2013, 34 (5), 193-9; and Read, S., et al., Blockade of CTLA-4 on CD4+CD25+ regulatory T cells abrogates their function in vivo. Journal of immunology 2006, 177 (7), 4376-83). Thus, to resolve the toxicity of the inhibitors, it is desirable to target the inhibitors to those cells that are important for tumor treatment but also suppressed by the checkpoint. Such targeting also has the potential to boost the efficacy of the inhibitors because it concentrates the inhibitors to those cells targeted for cancer therapy, whereas the current non-specific blockade wastes inhibitors in tumor treatment-unrelated interactions. Recently, a platelet-based carrier was used to target an immune checkpoint inhibitor, anti-PD-L1 antibody, to tumors, which resulted in better prevention of tumor recurrence under a post-surgery setting (Wang, C., et al., In situ activation of platelets with checkpoint inhibitors for post-surgical cancer immunotherapy. Nature Biomedical Engineering 2017, 1, 0011). However, it is unclear whether the carrier reduced the toxicity of the immune checkpoint inhibitors. Thus, drug carriers that can target immune checkpoint inhibitors and reduce their toxicity are needed.

Patients with advanced melanoma suffered a five-year survival rate of 16.6% due to the lack of an effective therapy (Howlader N, et al. SEER Cancer Statistics Review, 1975-2012. National Cancer Institute, 2015). Among the new therapies developed, the αPD-1 therapy has achieved lasting responses in some patients (Brahmer J R, et al. Safety and activity of anti-PD-L1 antibody in patients with advanced cancer. The New England journal of medicine. 2012; 366(26):2455-65; Hamid O, et al. Safety and tumor responses with lambrolizumab (anti-PD-1) in melanoma. The New England journal of medicine. 2013; 369(2):134-44; and Herbst R S, et al. Predictive correlates of response to the anti-PD-L1 antibody MPDL3280A in cancer patients. Nature. 2014; 515(7528):563-7). Two αPD-1s, Keytruda® and Opdivo®, have been approved for treating advanced melanoma; however, deficiencies exist in the current αPD-1 therapy. Among melanoma patients who were selected for αPD-1 clinical trials, response rates were less than 50% (Brahmer J R, et al. Safety and activity of anti-PD-L1 antibody in patients with advanced cancer. The New England journal of medicine. 2012; 366(26):2455-65; Hamid 0, et al. Safety and tumor responses with lambrolizumab (anti-PD-1) in melanoma. The New England journal of medicine. 2013; 369(2):134-44; and Herbst R S, et al. Predictive correlates of response to the anti-PD-L1 antibody MPDL3280A in cancer patients. Nature. 2014; 515(7528):563-7). Among the treated patients, 15% of them reported high grade (grades 3-4) treatment-related toxicity (Larkin J, et al. Combined Nivolumab and Ipilimumab or Monotherapy in Untreated Melanoma. The New England journal of medicine. 2015; and Topalian S L, et al. Safety, activity, and immune correlates of anti-PD-1 antibody in cancer. The New England journal of medicine. 2012; 366(26):2443-54), and 5-7% of them had to discontinue the treatment because they were never able to recover from the toxicity (Larkin J, et al. Combined Nivolumab and Ipilimumab or Monotherapy in Untreated Melanoma. The New England journal of medicine. 2015; and Topalian S L, et al. Safety, activity, and immune correlates of anti-PD-1 antibody in cancer. The New England journal of medicine. 2012; 366(26):2443-54). Even more alarming is that 4% of treated patients died from the toxicity (Topalian S L, et al. Safety, activity, and immune correlates of anti-PD-1 antibody in cancer. The New England journal of medicine. 2012; 366(26):2443-54). These numbers highlight the importance of reducing the toxicity of αPD-1 therapy and reveal the ineffectiveness of current αPD-1 toxicity mitigation methods. Furthermore, the current mitigation methods, primarily non-specific immune suppression, exposed the treated patients to immune deficiency and serious infections (Tarhini A. Immune-mediated adverse events associated with ipilimumab ctla-4 blockade therapy: the underlying mechanisms and clinical management. Scientifica (Cairo). 2013; 2013:857519). Patients with autoimmune disorders and chronic infections were excluded from αPD-1 clinical trials because they are sensitive to the αPD-1 toxicity (Robbins P F, et al. Mining exomic sequencing data to identify mutated antigens recognized by adoptively transferred tumor-reactive T cells. Nature medicine. 2013; 19(6):747-52). Although the exact exclusion rates were never published, records at the University of Utah Hospital show that 5% melanoma patients reported autoimmune disorders (unpublished data). In addition, the fact that ˜50 million Americans have autoimmune disorders hints at the possible scope of the exclusion (American Autoimmune Related Diseases Association I. Autoimmune Statistics 2015. Available from: https://www.aarda.org/autoimmune-information/autoimmune-statistics/). Furthermore, the exclusion may miss those patients who could respond to the therapy better than other patients. For example, melanoma patients with vitiligo, an autoimmune disorder, actually respond better to immunotherapy (Uchi H, S et al. Unraveling the complex relationship between cancer immunity and autoimmunity: lessons from melanoma and vitiligo. Advances in immunology. 2006; 90:215-41). These deficiencies can be attributed to, for example, the indiscriminate PD-1 blockade of the current therapy. To resolve these deficiencies, a targeted anti-PD-1 antibody (αPD-1) therapy that enacts a cell-specific PD-1 blockade was developed and described herein.

In some aspects, the anti-programmed death-1 antibody (αPD-1) was used as a model immune checkpoint inhibitor. Some of the Examples describe the generation of a recombinant single-chain variable fragment (scFv) of αPD-1 (and α-CTLA-4).

In an aspect, the targeting can be a scFv of cytotoxic T-lymphocyte-associated protein 4 (CTLA-4). In an aspect, the targeting moiety can be derived from an anti-CTLA-4 antibody. In an aspect, the targeting moiety can be an antibody. The antibody can be an anti-CTLA-4 antibody. Examples of anti-CTLA-4 antibodies include but are not limited to ipilimumab, tremelimumab and UC10-4F10 clone. In an aspect, the anti-CTLA-4 antibody can be ipilimumab.

In an aspect, a fusion protein comprises a targeting moiety, wherein the targeting moiety can be a single chain variable fragment (scFv) of an anti-PD-1 antibody; a plasma protein binding domain, wherein the plasma binding domain is an albumin-binding protein domain; and a toxin or biological variant thereof, wherein the toxin is a Pseudomonas exotoxin or a biological variant thereof.

In an aspect, the fusion protein comprises, from the N-terminus to the C-terminus, a single chain variable fragment (scFv) of an anti-PD-1 antibody, a peptide linker, an albumin-binding protein domain, a second peptide linker, and a Pseudomonas exotoxin or biological variant thereof.

Linkers.

Disclosed herein, are fusion proteins as described herein, further comprising one or more linkers. The linkers can be of any length, of a flexible sequence and not have any charges. Examples of linkers that can be useful in the present compositions can be found in “Fusion protein linkers: Property, design and functionality” 2013; Advanced Drug Delivery Reviews, Volume 65, Pages 1357-1369 which is incorporated by reference herein in its entirety. In an aspect, the one or more linkers are peptide-based. In an aspect, the one or more linkers can be GGGGSGGGGSGGGGS (SEQ ID NO: 1), GGGGS (SEQ ID NO: 2), GGGGSGGGGS (SEQ ID NO: 3), or GGGGSGGGGSGGGGSGGGGS (SEQ ID NO: 4).

Sites available for linking can be found on the fusion proteins described herein. Useful linkers in the present compositions can comprise a group that is reactive with a primary amine on the fusion protein to which a targeting moiety or toxin can be conjugated. Useful linkers are available from commercial sources. One of ordinary skill in the art is capable of selecting the appropriate linker.

The linker can be a covalent bond. To form covalent bonds, a chemically reactive group can be used, for instance, that has a wide variety of active carboxyl groups (e.g., esters) where the hydroxyl moiety is physiologically acceptable at the levels required to modify the fusion protein.

Any of the fusion proteins described herein and its components (e.g., targeting moiety, plasma protein binding domain and toxin) can be modified to chemically interact with, or to include, a linker as described herein. These modified fusion proteins and fusion protein-linker constructs are within the scope of the present disclosure and can be packaged as a component of a kit with instructions for completing the process of conjugation to a toxin. The fusion proteins can be modified to include a cysteine residue or other thio-bearing moiety (e.g., C-SH) at the N-terminus, C-terminus, or both.

In an aspect, the fusion proteins described herein comprise a linker between the targeting moiety and the plasma protein binding domain. In an aspect, the fusion proteins described herein can comprise a linker between the plasma protein binding domain and the toxin.

Toxin.

A wide variety of toxic (e.g., cytotoxic) agents can be incorporated into the fusion proteins disclosed herein. The toxin or biological variant thereof can be an immunogenic toxin. The toxin can be a protein or a small molecule. In an aspect, the immunogenic toxin can be a bacteria or plant toxin. In some aspects, the immunogenic toxin can be a bacterial toxin. Examples of bacterial toxins include but are not limited to Pseudomonas exotoxin and Diphtheria toxin, or a biological variant thereof. In some aspects, the immunogenic toxin can be a plant toxin. Examples of plant toxins include but are not limited to ricin (P02878), saporin (P20656), Bouganin (Q8W4U4), and gelonin (P33186) or a biological variant thereof. The toxins can be modified. In an aspect, the toxin is a fragment of a bacterial or plant toxin. The toxins disclosed herein can be at least 20%, 25%, 30%, 35%, 40%, 45%, 50%, 55%, 60%, 65%, 70%, 75%, 80%, 85%, 90%, 95%, or 98% identical to a reference toxin. For example, a toxin within a fusion protein can have an amino acid sequence that is at least 20% identical to the wild-type sequence.

In an aspect, the toxin can be Pseudomonas exotoxin (PE) or a biological variant thereof. Native PE consists of three structural domains. Domain I consists of Domain Ia (residues 1-252) and Domain Ib (residues 365-404); Domain II (residues 253-364); and Domain III (residues 405-613). In an aspect, the PE toxin can be a modified. The PE toxin useful in the fusion protein described herein can include residues 274-284 and 395-613. In some aspects, the PE toxin can exclude residues 1-273 and 285-394. Functional information and the protein sequence of Pseudomonas aeruginosa Exotoxin A can be found using the UniProtKB database and P11439 as the entry. In an aspect, the PE toxin or biological variant comprises:

(SEQ ID NO: 5)
RHRQPRGWEQLPTGAEFLGDGGDVSFSTRGTQNWTVERLLQAHRQLEERGY
VFVGYHGTFLEAAQSIVFGGVRARSQDLDAIWRGFYIAGDPALAYGYAQDQ
EPDARGRIRNGALLRVYVPRSSLPGFYRTSLTLAAPEAAGEVERLIGHPLP
LRLDAITGPEEEGGRLETILGWPLAERTVVIPSAIPTDPRNVGGDLDPSSI
PDKEQAISALPDYASQPGKPPREDLK.

In an aspect, the toxin can be variant of PE. Examples of PE variants include but are not limited to LMB-100, LMB-T20 and LMB-T14 as well as the PE variants disclosed in Strategies to Reduce the Immunogenicity of Recombinant Immunotoxin. The American Journal of Pathology, Vol. 188, No. 8, August 2018, which is hereby incorporated by reference in its entirtity for teaching the same.

Functional information and the protein sequence of Diphtheria toxin can be found using the UniProtKB database and P00588 as the entry.

Examples of other toxins that can be used in the fusion protein disclosed herein can include human-originated recombinant toxins, such as human granzyme B (UniProtKB—P10144), soluble tumor necrosis factor-related apoptosis-inducing ligand (sTRAIL, P50591), and pancreatic RNase I (P07998).

Plasma Protein Binding Domain.

The fusion protein disclosed herein can bind to a plasma protein binding domain. In an aspect, the plasma protein binding domain can be an albumin binding protein domain. In an aspect, the plasma protein binding domain can be an immunoglobulin protein binding domain. In an aspect, the immunoglobulin protein binding domain can be IgG.

The fusion protein disclosed herein can bind to serum (or plasma) albumin. In an aspect, the albumin binding protein domain can be LAEAKVLANRELDKYGVSDFYKRLINKAKTVEGVEALKLHILAALP (SEQ ID NO: 6).

The plasma protein binding domain can be modified by methods known to one of ordinary skill in the art. In an aspect, the albumin binding protein domain can be modified. In an aspect, the albumin binding protein domain can be LAEAKVLANRELDKYGVSDYYKNLINNAKTVEGVKALIDEILAALP (SEQ ID NO: 13); LAEAKVLANRELDKYGVSDYYKNIINRAKTVEGVRALKLHILAALP (SEQ ID NO: 14); or LAEAKVLANRELDKYGVSDYYKNLINKAKTVEGVEALTLHILAALP (SEQ ID NO: 15). Modifications of the albumin binding protein domain can be found in Jonsson et al.; “Engineering of a femtomolar affinity binding protein to human serum albumin;” Protein Engineering, Design & Selection 21(8); (2008):515-527 which is incorporated by reference herein in its entirety.

The fusion protein can bind to or otherwise associate with serum albumin in such a way that it exhibits an increased serum half-life. In an aspect, the toxin (e.g., PE; when combined or a part of the fusion protein as described herein) can have an increased serum half-life when compared to the administration of the toxin (e.g., PE) alone or not as part of the fusion protein. In an aspect, the serum half-life of the toxin (e.g., PE; when combined or a part of the fusion protein as described herein) can be increased by at least 5-fold when compared to the administration of the toxin (e.g., PE) alone or not a part of the fusion protein. In an aspect, the serum half-life of the toxin can be increased by about between 5 to 60 fold when compared to the administration of the toxin alone or not a part of the fusion protein. In an aspect, the serum half-life of the toxin (e.g., PE; when combined or a part of the fusion protein as described herein) can be increased from 2 hours to 80 hours longer or any time in between when compared to the administration of the toxin (e.g., PE) alone or not as part of the fusion protein. In an aspect, the serum half-life of the toxin (e.g., PE; when combined or a part of the fusion protein as described herein) can be increased from 15 hours to 80 hours longer or any time in between when compared to the administration of the toxin (e.g., PE) alone or not as part of the fusion protein. For example, PE that is not bound the albumin binding protein domain has a half-life of about 1.3 hours, while PE that is part of the fusion protein disclosed herein has a half-life of about 80 hours.

The half-life of a composition can generally be defined as the time taken for the serum concentration of the composition to be reduced by 50%, in vivo, for example, due to degradation of the composition and/or clearance or sequestration of the compound by natural or other mechanisms. The half-life of the fusion protein or any of its components can be determined in any manner known in the art including by pharmacokinetic analysis.

Methods of Making Fusion Proteins

Disclosed herein are techniques that can be used to produce the fusion proteins described herein.

Targeting Moiety and Plasma Protein Binding Domain.

In some aspects, the targeting moiety and plasma protein binding domain can be engineered along with the targeting moiety (e.g., anti-PD-1 antibody) into a single protein (i.e., a single fusion protein).

Fusion proteins can be made using recombinant DNA. DNA encoding fusion proteins (targeting moiety and plasma protein binding domain) can be readily isolated and sequenced using conventional procedures (e.g., by using oligonucleotide probes that are capable of binding specifically to genes encoding the heavy and light chains of murine antibodies). The DNA can be inserted into E. coli via transformation. The transformed E. coli then can produce the fusion proteins. The proteins can be purified from the cell lysate of E. coli.

Antibodies.

As noted above, the fusion proteins as disclosed herein, can include an antibody or fragment thereof, a single chain variable fragment (scFv) of an antibody or a Fab′ fragment or a biologically active variant thereof. As is well known in the art, monoclonal antibodies can be made by recombinant DNA. DNA encoding monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques.

In vitro methods are also suitable for preparing monovalent antibodies. As it is well known in the art, some types of antibody fragments can be produced through enzymatic treatment of a full-length antibody. Digestion of antibodies to produce fragments thereof, particularly, Fab fragments, can be accomplished using routine techniques known in the art. For instance, digestion can be performed using papain. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen. Antibodies incorporated into the present bi-functional allosteric protein-drug molecules can be generated by digestion with these enzymes or produced by other methods.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment can be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment.

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen binding portion of an antibody) which contains a portion of an antigen binding site from a non-human (donor) antibody integrated into the framework of a human (recipient) antibody.

The Fv region is a minimal fragment containing a complete antigen-recognition and binding site consisting of one heavy chain and one light chain variable domain. The three CDRs of each variable domain interact to define an antigen-biding site on the surface of the Vh-Vl dimer. Collectively, the six CDRs confer antigen-binding specificity to the antibody. As well known in the art, a “single-chain” antibody or “scFv” fragment is a single chain Fv variant formed when the VH and Vl domains of an antibody are included in a single polypeptide chain that recognizes and binds an antigen. Typically, single-chain antibodies include a polypeptide linker between the Vh and Vl domains that enables the scFv to form a desired three-dimensional structure for antigen binding.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies can also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody.

Methods for humanizing non-human antibodies are well known in the art. For example, humanized antibodies can be generated by substituting rodent CDRs or CDR sequences for the corresponding sequences of a human antibody. Methods that can be used to produce humanized antibodies are also well known in the art.

Toxin.

In some aspects, the toxin (if it is protein toxin) can be engineered in the same way as other components of the fusion protein. Briefly, toxin proteins can be made by recombinant DNA. DNA encoding fusion proteins can be readily isolated and sequenced using conventional procedures (e.g., by using oligonucleotide probes that are capable of binding specifically to genes encoding the heavy and light chains of murine antibodies). The DNA can be inserted into E. coli via transformation. The transformed E. coli can produce the fusion proteins. The proteins can be purified from the cell lysate of E. coli.

Configurations.

Each part of a given fusion protein, including the targeting moiety, plasma protein binding domain and toxin, can be selected independently. One of ordinary skill in the art would understand that the component parts need to be associated in a compatible manner. The fusion protein can be used to deliver a toxin (e.g., cytotoxic agent) to a patient for the treatment of an autoimmune disease, disorder or Type I diabetes, inducing apoptosis or halting or preventing the cell death of one or more lymphocytes (e.g., T cells or B cells). In an aspect, the fusion protein can further include a detectable marker. With the inclusion of a detectable marker, such as fluorescence or peptide probes, the fusion protein as described herein can also be used to map the distribution of targets to which the targeting moieties bind.

Pharmaceutical Compositions

As disclosed herein, are pharmaceutical compositions, comprising the fusion protein and a pharmaceutical acceptable carrier described above. In some aspects, the targeting moiety can be a single chain variable fragment (scFv) of an anti-PD-1, the plasma protein binding domain is an albumin-binding protein domain and the toxin is a Pseudomonas exotoxin or a biological variant thereof and the pharmaceutical composition is formulated for intravenous administration. The compositions of the present disclosure also contain a therapeutically effective amount of a fusion protein as described herein. The compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used to deliver the fusion proteins. Thus, compositions can be prepared for parenteral administration that includes fusion proteins dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions are formulated for application to the skin or to a mucosal surface, one or more of the excipients can be a solvent or emulsifier for the formulation of a cream, an ointment, and the like.

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

Methods of Treatment

Disclosed herein, are methods of treating a subject with an autoimmune disease, the method comprising: (a) identifying a subject in need of treatment; and (b) administering to the subject a therapeutically effective amount of the pharmaceutical composition comprising fusion proteins comprising a targeting moiety, a plasma protein binding domain, and a toxin or biological variant thereof, and a pharmaceutically acceptable carrier.

Disclosed herein, are methods of treating or ameliorating a symptom of Type I diabetes mellitus in a subject, the method comprising: (a) identifying a subject in need of treatment; and (b) administering to the subject a therapeutically effective amount of the pharmaceutical composition comprising fusion proteins comprising a targeting moiety, a plasma protein binding domain, and a toxin or biological variant thereof, and a pharmaceutically acceptable carrier.

Disclosed herein, are methods of preventing or delaying the onset of Type I diabetes mellitus in a subject at risk for developing Type I diabetes, the method comprising: (a) identifying a subject in need of treatment; and (b) administering to the subject a therapeutically effective amount of the pharmaceutical composition comprising fusion proteins comprising a targeting moiety, a plasma protein binding domain, and a toxin or biological variant thereof, and a pharmaceutically acceptable carrier.

Disclosed herein, are methods of inducing apoptosis, the method comprising: contacting a cell with a composition comprising a fusion protein, wherein the fusion protein comprises a single chain variable fragment (scFv) of an anti-PD-1 antibody, a plasma protein binding domain and a toxin or a biological variant thereof wherein the contacting of the cells with the composition induces apoptosis. In an aspect, the method can include contacting a population of cells. In an aspect, the single chain variable fragment (scFv) of the anti-PD-1-antibody can be an scFV of nivolumab, pembrolizumab, pidilizumab, MEDI0680, BMS-936559, clone J116, or a biologically active variant thereof. In an aspect, the plasma protein binding domain can be an albumin-binding protein domain. In an aspect, the toxin can be Pseudomonas exotoxin or a biologically active variant thereof. In an aspect, the step of contacting the cells with the composition can be repeated. In an aspect, the cell can express PD-1. In an aspect, the cell or population of cells can be in a subject. In an aspect, the subject has Type I diabetes, multiple sclerosis, arthritis or can be undergoing an organ transplant. In an aspect, the fusion protein disclosed herein can be used to treat subjects undergoing an organ transplant to prevent acute organ rejection. The fusion protein disclosed herein can be administered before (minutes, hours or days) and during the transplantation.

In an aspect, the anti-PD-1 antibody can selectively bind to a PD-1 cell surface receptor. In an aspect, the method further comprises internalization of the fusion protein by a cell expressing a PD-1 cell surface receptor.

Disclosed herein, are methods of inducing apoptosis, the method comprising: contacting a cell with a composition comprising a fusion protein, wherein the fusion protein comprises a single chain variable fragment (scFv) of an anti-CTLA-4 antibody, a plasma protein binding domain and a toxin or a biological variant thereof wherein the contacting of the cells with the composition induces apoptosis. In an aspect, the method can include contacting a population of cells. In an aspect, the single chain variable fragment (scFv) of the anti-CTLA-4 antibody can be an scFV of ipilimumab, or a biologically active variant thereof. In an aspect, the plasma protein binding domain can be an albumin-binding protein domain. In an aspect, the toxin can be Pseudomonas exotoxin or a biologically active variant thereof. In an aspect, the step of contacting the cells with the composition can be repeated. In an aspect, the cell can express CTLA-4. In an aspect, the cell or population of cells can be in a subject. In an aspect, the subject has Type I diabetes, multiple sclerosis, arthritis or can be undergoing an organ transplant. In an aspect, the anti-CTLA-4 antibody can selectively bind to a CTLA-4 cell surface receptor. In an aspect, the method further comprises internalization of the fusion protein by a cell expressing a CTLA-4 cell surface receptor. In an aspect, the cell can express CTLA-4. In an aspect, the anti-CTLA-4 antibody can selectively bind to a CTLA-4 cell surface receptor.

In an aspect, skilled person can determine an efficacious dose, an efficacious schedule, or an efficacious route of administration for a disclosed composition or a disclosed fusion protein so as to induce apoptosis.

In an aspect, any of disclosed methods of inducing apoptosis can comprise confirming apoptosis of the cells. Methods of confirming apoptosis are known to the art and include, but are not limited to: measuring caspase-3 activity, measuring annexin V/propidium iodine binding, and measuring terminal deoxynucleotidyl transferase dUTP nick end-labeling. In an aspect, confirming apoptosis can comprise one of the following: measuring caspase-3 activity, measuring annexin/propidium iodine binding, and measuring terminal deoxynucleotidyl transferase dUTP nick end-labeling. In an aspect, confirming apoptosis can comprise two of the following: measuring caspase-3 activity, measuring annexin V/propidium iodine binding, and measuring terminal deoxynucleotidyl transferase dUTP nick end-labeling. In an aspect, confirming apoptosis can comprise all of the following: measuring caspase-3 activity, measuring annexin V/propidium iodine binding, and measuring terminal deoxynucleotidyl transferase dUTP nick end labeling.

In an aspect of a disclosed method of inducing apoptosis, the cell or population of cells can be B cells or T cells.

Disclosed herein, are methods of preventing or halting cell death of one or more pancreatic islets, the method comprising: contacting one or more lymphocytes with a composition comprising a fusion protein, wherein the fusion protein comprises a single chain variable fragment (scFv) of an anti-PD-1 antibody, a plasma protein binding domain and a toxin or a biological variant thereof; wherein the contacting of the one or more lymphocytes with the composition prevents or halts cell death of one or more pancreatic islets. In an aspect, the method can include contacting a population of cells (e.g., lymphocytes). In an aspect, the one or more lymphocytes can be T cells or B cells or both T cells and B cells. In an aspect, the pancreatic islets can be beta cells, alpha cells, delta cells, gamma cells and/or epsilon cells or any combination of pancreatic islets. In an aspect, the T cell lymphocytes or B cell lymphocytes are not depleted. In an aspect, the quantity, relative abundance, fraction or number of lymphocytes can be determined. In an aspect, the quantity, relative abundance, fraction or number of lymphocytes can be determined to be unchanged compared to a control or reference sample.

In an aspect of any of the disclosed methods herein, the composition or fusion protein described herein can be combined with one or more additional therapies. In an aspect, the fusion protein can be administered alone or in combination with other biologically active agents into compositions suitable for administration to a subject. In an aspect, the fusion protein can be administered in combination with a therapeutic agent. In an aspect, methods directed to treating subjects with Type I diabetes mellitus or at risk for developing Type I diabetes mellitus, the compositions or fusion proteins disclosed herein can be combined with, for example, insulin, pancreatic islet cell transplantation, T regulatory cell transplantation or vaccines that induce immune tolerance such as aDiamyd and DiaPep277. In an aspect, methods directed to treating subjects with Multiple Sclerosis or at risk for developing Multiple Sclerosis, the compositions or fusion proteins disclosed herein can be combined with, for example, IFN-beta (e.g., Avonex, Betaseron) or Glatiramer acetate (Copaxone). In an aspect, methods directed to treating subjects with rheumatoid arthritis or at risk for developing rheumatoid arthritis, the compositions or fusion proteins disclosed herein can be combined with, for example, anti-malarials, leflunpmide or cyclophosphamide. The combined therapy can be administered as a co-formulation, or separately. When administered separately, the combined therapy can be administered simultaneously or sequentially. The formulations can be made using methods routine in the art and particular guidance may be provided by prior formulations of protein-based or small molecule-based therapeutics.

The pharmaceutical compositions described above can be formulated to include a therapeutically effective amount of a fusion protein as disclosed herein. Therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to one or more autoimmune diseases or where the patient has a clinically determined predisposition or increased susceptibility (in some cases, a greatly increased susceptibility) to Type I diabetes.

The pharmaceutical compositions described herein can be administered to the subject (e.g., a human subject or human patient) in an amount sufficient to delay, reduce, or preferably prevent the onset of clinical disease. Accordingly, in some aspects, the subject is a human subject. In therapeutic applications, compositions are administered to a subject (e.g., a human subject) already with or diagnosed with an autoimmune disease in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a pharmaceutical composition can be an amount that achieves a cure, but that outcome is only one among several that can be achieved. As noted, a therapeutically effective amount includes amounts that provide a treatment in which the onset or progression of the autoimmune disease is delayed, hindered, or prevented, or the autoimmune disease or a symptom of the autoimmune disease is ameliorated. One or more of the symptoms can be less severe. Recovery can be accelerated in an individual who has been treated.

In some aspects, the autoimmune disease can be non-Hodgkin's lymphoma, rheumatoid arthritis, chronic lymphocytic leukemia, multiple sclerosis, systemic lupus erythematosus, autoimmune hemolytic anemia, pure red cell aplasia, idiopathic thrombocytopenic purpura, Evans syndrome, vasculitis, bullous skin disorders, type 1 diabetes mellitus, Sjögren's syndrome, Devic's disease, or Graves' disease ophthalmopathy. In other aspects, the autoimmune disease can be Type I diabetes mellitus, multiple sclerosis, or rheumatoid arthritis. In an aspect, the autoimmune disease is Type I diabetes mellitus.

Disclosed herein, are methods of treating a subject with Type I diabetes mellitus.

Disclosed herein, are methods of treating a subject with multiple sclerosis.

Amounts effective for this use can depend on the severity of the autoimmune disease and the weight and general state and health of the subject. Suitable regimes for initial administration and booster administrations are typified by an initial administration followed by repeated doses at one or more hourly, daily, weekly, or monthly intervals by a subsequent administration. For example, a subject can receive a fusion protein one or more times per week (e.g., 2, 3, 4, 5, 6, or 7 or more times per week).

The total effective amount of a fusion protein in the pharmaceutical compositions disclosed herein can be administered to a mammal as a single dose, either as a bolus or by infusion over a relatively short period of time, or can be administered using a fractionated treatment protocol in which multiple doses are administered over a more prolonged period of time (e.g., a dose every 4-6, 8-12, 14-16, or 18-24 hours, or every 2-4 days, 1-2 weeks, or once a month). Alternatively, continuous intravenous infusions sufficient to maintain therapeutically effective concentrations in the blood are also within the scope of the present disclosure.

The therapeutically effective amount of the toxins (or cytotoxic agents) present within the compositions described herein and used in the methods as disclosed herein applied to mammals (e.g., humans) can be determined by one of ordinary skill in the art with consideration of individual differences in age, weight, and other general conditions (as mentioned above). Because the fusion proteins of the present disclosure can be stable in serum and the bloodstream and in some cases more specific, the dosage of the fusion protein including any individual component can be lower (or higher) than an effective dose of any of the individual components when unbound. Accordingly, in some aspects, the toxin administered can have an increased efficacy or reduced side effects when administered as part of a fusion protein as compared to when the toxin is administered alone or not as part of a fusion protein.

Kits

Disclosed herein are kits comprising a targeting moiety, a plasma protein binding domain, and a toxin or biological variant thereof. In an aspect, the disclosed kits can comprise instructions for preparing the fusion proteins and administering fusion proteins comprising a targeting moiety, a plasma protein binding domain, and a toxin or biological variant thereof.

In an aspect, the kits can comprise fusion proteins wherein the targeting moiety is single chain variable fragment (scFv) of an anti-PD-1 antibody, the plasma protein binding domain is an albumin-binding protein domain and the toxin is Pseudomonas exotoxin or a biological variant thereof. In other aspects, the kits can further comprise a first linker between the targeting moiety and the plasma protein binding domain and a second linker between the plasma protein binding domain and the toxin or biological variant thereof. The linker can be a peptide. In an aspect, the first and second linker is a peptide.

### EXAMPLES

### Example 1: Synthesis of the Fusion Protein

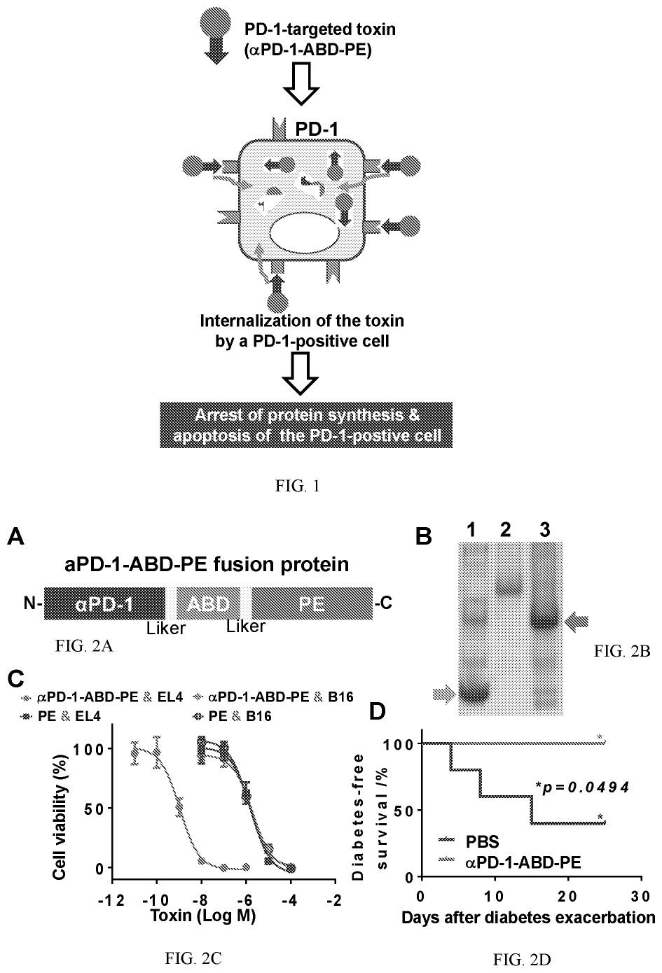

The fusion protein comprises at least three functional elements, a single-chain variable fragment (scFv) of anti-PD-1 antibody (αPD-1), an albumin-binding protein domain (ABD)[11-13], and a re-engineered (or modified) Pseudomonas exotoxin (PE) (FIG. 1; FIG. 2A). The αPD-1 scFv can target PD-1-positive cells; the ABD, by complexing with serum albumin, can extend the in vivo half-life of the fusion (Jonsson, A., et al., Engineering of a femtomolar affinity binding protein to human serum albumin. Protein Eng Des Sel, 2008. 21(8): p. 515-27; Levy, O. E., et al., Novel exenatide analogs with peptidic albumin binding domains: potent anti-diabetic agents with extended duration of action. PLoS One, 2014. 9(2): p. e87704; and Orlova, A., et al., Site-specific radiometal labeling and improved biodistribution using ABY-027, a novel HER2-targeting affibody molecule-albumin-binding domain fusion protein. J Nucl Med, 2013. 54(6): p. 961-8) protein. The re-engineered PE has specific toxicity to the cells that it enters (Pastan, I., et al., Immunotoxins with decreased immunogenicity and improved activity. Leuk Lymphoma, 2011. 52 Suppl 2: p. 87-90; and Weldon, J. E. and I. Pastan, A guide to taming a toxin-recombinant immunotoxins constructed from Pseudomonas exotoxin A for the treatment of cancer. Febs j, 2011. 278(23): p. 4683-700) and is significantly less immunogenic than the original PE (Weldon, J. E. and I. Pastan, A guide to taming a toxin-recombinant immunotoxins constructed from Pseudomonas exotoxin A for the treatment of cancer. Febs j, 2011. 278(23): p. 4683-700; Liu, W., et al., Recombinant immunotoxin engineered for low immunogenicity and antigenicity by identifying and silencing human B-cell epitopes. Proc Natl Acad Sci USA, 2012. 109(29): p. 11782-7; Mazor, R., et al., Identification and elimination of an immunodominant T-cell epitope in recombinant immunotoxins based on Pseudomonas exotoxin A. Proc Natl Acad Sci USA, 2012. 109(51): p. E3597-603; and Mazor, R., et al., Recombinant Immunotoxin with T-cell Epitope Mutations That Greatly Reduce Immunogenicity for Treatment of Mesothelin-Expressing Tumors. Mol Cancer Ther, 2015. 14(12): p. 2789-96. The data show that the fusion protein (e.g., αPD-1-ABD-PE), (1) has selective toxicity to PD-1-positive cells; (2) binds with mouse albumin; (3) prevents non-obese diabetes (NOD) mice, a strain prone to diabetes, from developing diabetes. Based on the above scientific premise and these data, it was tested whether αPD-1-ABD-PE will stop autoimmune destruction to β-cells by selectively depleting T or B lymphocytes.

Targeted PD-1 positive cells were predicted to lead to the depletion of said cells but also the reversal of T1D along with fewer or more mild side effects because (1) a blockade of the PD-1 immune checkpoint exacerbates diabetes in murine models and patients, thus, PD-1-positive cells play an important role in diabetes progression (Okamoto, M., et al., Fulminant type 1 diabetes mellitus with anti programmed cell death-I therapy. J Diabetes Investig, 2016. 7(6): p. 915-918; Hughes, J., et al., Precipitation of Autoimmune Diabetes With Anti-PD-1 Immunotherapy. Diabetes Care, 2015. 38(4): p. e55; and Ansari, M. J., et al., The programmed death-I (PD-1) pathway regulates autoimmune diabetes in nonobese diabetic (NOD) mice. J Exp Med, 2003. 198(1): p. 63-9; (2) PD-1-positive cells include both T and B lymphocytes; (3) PD-1 is selectively expressed in activated immune cells; thus, PD-1-targeted depletion will not affect naive cells or compromise immune defense against subsequent infections and malignancy; (4) αPD-1-ABD-PE completely prevented diabetes occurrence in NOD mice under an experimental condition that precipitates diabetes in these mice (FIG. 1); (5) there was no apparent toxicity during the above αPD-1-ABD-PE treatment.

Thus, the development of an effective, low-toxicity treatment to stop autoimmune destruction to β-cells is needed. PD-1-targeted depletion using the αPD-1-ABD-PE fusion can meet this need.

The following have been generated αPD-1-ABD-PE, αPD-1-PE, ABD-PE, PE, and the αPD-1 scFv. The data show that αPD-1-ABD-PE is functional in vitro and in vivo (see FIG. 2).

### Example 2: αPD-1-ABD-PE is Internalized by PD-1 Positive Cells and Selectively Toxic to the Cells

Confirmation that αPD-1-ABD-PE is internalized by PD-1-positive cells and selectively toxic to the cells will be performed. It is expected that (1) PD-1-positive cells internalize αPD-1-ABD-PE (see, FIG. 2C and below) and (2) the cytotoxic IC50 of αPD-1-ABD-PE to PD-1-positive cells is 1000 times smaller than the IC50 to PD-1-negative cells. The results will underscore the selectivity and the safety of using αPD-1-ABD-PE to deplete PD-1-positive cells.

To examine selective internalization, αPD-1-ABD-PE, its negative controls (ABD-PE and PE), and its positive control (αPD-1) will be labeled with FITC; and then these samples will be incubated with PD-1-positive cells (e.g. EL4 cells) and PD-1-negative cells (e.g. 2H11 endothelial or B16 melanoma cells). Next, the uptake of the samples by the cells will be compared using flow cytometry. It is expected that (1) PD-1-positive cells will internalize more αPD-1-ABD-PE and αPD-1 than the negative controls and (2) PD-1-negative cells will internalize much less αPD-1-ABD-PE as compared to the positive cells.

To examine the selective toxicity, the above mentioned samples will be incubated with PD-1-positive and PD-1-negative cells for 48 hours and then the MTS assay will be used to check cell viability. In addition to the aforementioned, it is also expected that ABD-PE and PE will have equally low toxicity to both PD-1-positive and PD-1-negative cells.

FIG. 2C shows that αPD-1-ABD-PE is cytotoxic to PD-1-positive cells (EL4) compared to control and PD-1-negative cells. The IC50s of the four sample/cell pairs are: αPD-1-ABD-PE and EL4 (1.1 nm, 95% CI: 0.8˜1.3 nm), αPD-1-ABD-PE and B16 (1.9 μm, 95% CI: 1.7˜2.2 μm), PE and EL4 (1.6 μm, 95% CI: 1.3˜2.1 μm), and PE and B16 (1.3 μm, 95% CI 1.1˜1.5 μm).

FIG. 2D shows that αPD-1-ABD-PE prevented T1D development in NOD mice. 10-week old NOD mice were randomly assigned into two groups to receive 5 doses of PBS or αPD-1-ABD-PE (5 mg/Kg body weight). Then, both groups were exacerbated for T1D development with 5 doses of αPD-1 (12.5 mg/Kg body weight). The median diabetes-free survival time of two groups after the αPD-1 treatment was compared using the Log-Rank test and found significantly different. None of the αPD-1-ABD-PE-treated mice developed T1D up to 30 days after the exacerbation treatment.

### Example 3: ABD Improves In Vivo Half-Life of αPD-1-ABD-PE

Experiments will be carried out to validate that ABD improves in vivo half-life of αPD-1-ABD-PE. It is expected that αPD-1-ABD-PE will have a significant longer plasma half-live than its control, αPD-1-PE. This expectation is based on the finding that αPD-1-ABD-PE bound with albumin (FIG. 2B) as well as other molecules bound with albumin have longer half-lives (Muller, D., et al., Improved pharmacokinetics of recombinant bispecific antibody molecules by fusion to human serum albumin. J Biol Chem, 2007. 282(17): p. 12650-60). This result will demonstrate the importance of ABD in αPD-1-ABD-PE. A pharmacokinetics (PK) study will be performed for this validation. Specifically, mice will be intraperitoneally injected with Alexa680-labelled αPD-1-ABD-PE or its negative controls (αPD-1-PE and PE) at a dose 5 mg/kg. Blood will be withdrawn from each mouse at pre-set time points to monitor plasma concentration changes of each injected sample. Concentration changes will be fit over time to a two compartment extravascular PK model and the half-lives of each sample will be obtained through the fitting. Area under the curves (AUC) for each sample will reinforce the conclusion of PK study.

Confirmation that αPD-1-ABD-PE ABD has a long in vivo half-life will be done. The samples include Alexa680-labeled αPD-1-ABD-PE and αPD-1-PE. Based on the pharmacokinetic (PK) data shown in FIG. 2D, it is expected that αPD-1-ABD-PE has a significantly longer half-life than αPD-1-PE. To this end, mice will be injected intraperitoneally with the labeled αPD-1-ABD-PE or αPD-1-PE and the plasma concentrations of the injected samples will be checked at pre-set time points. Then, the concentration changes as performed to generate data in FIG. 4D and described in previous publications (Zhao P, Xia G, Dong S, Jiang Z X, Chen M. An iTEP-salinomycin nanoparticle that specifically and effectively inhibits metastases of 4T1 orthotopic breast tumors. Biomaterials. 2016; 93:1-9; and Zhao P, Dong S, Bhattacharyya J, Chen M. iTEP nanoparticle-delivered salinomycin displays an enhanced toxicity to cancer stem cells in orthotopic breast tumors. Mol Pharm. 2014; 11(8):2703-12) will be analyzed and the elimination half-lives of the samples will be obtained. The area under the curve (AUC) for each sample will be calculated to reinforce the conclusion of half-lives.

FIG. 4D shows that the elimination half-life of αPD-1-ABD-PE is 58 times of that of αPD-1-PE (n=3). The samples were injected intraperitoneally. This result demonstrates the importance of ABD in extending the half-life of αPD-1-ABD-PE. The following experimental procedure was followed: 5 nmole FITC-labeled αPD-1-PE or αPD-1-ABD-PE were injected via intraperitoneal injection. To measure PK, blood was withdrawn from mice at pre-defined time points with each time point of each repeat used one mouse (a total of 13 points with triplicates for each point, 13*3=39 mice total). Blood was collected and analyzed together by measuring FITC fluorescent emission at 521 nm when being excited by 494 nm light. The labeled protein concentration was calculated by fitting into a standard curve prepared using pure FITC. For αPD-1-PE: the half-life (h) was 1.3; Cmax (μmoL) was 0.6; and AUC (μmoL*h) was 1.2. For αPD-1-ABD-PE: the half-life (h) was 75.5; Cmax (μmoL) was 1.6; and AUC (μmoL*h) was 35.6.

### Example 4: Reverse T1D with αPD-1-ABD-PE without Abating T and B Lymphocyte Populations

Whether αPD-1-ABD-PE can reverse newly-diagnosed T1D will be examined. It is expected αPD-1-ABD-PE will reverse the newly-diagnosed T1D in NOD mice, a common T1D model (Shoda, L. K., et al., A comprehensive review of interventions in the NOD mouse and implications for translation. Immunity, 2005. 23(2): p. 115-26; and Zhou, Z., et al., Type 1 diabetes associated HLA-DQ2 and DQ8 molecules are relatively resistant to HLA-DM mediated release of invariant chain-derived CLIP peptides. European Journal of Immunology, 2016. 46(4): p. 834-845). This expectation is based on the findings that (1) αPD-1-ABD-PE is selective toxicity to PD-1-positive cells (FIG. 2B); (2) PD-1-positive cells are important to T1D progression (Okamoto, M., et al., Fulminant type 1 diabetes mellitus with anti programmed cell death-I therapy. J Diabetes Investig, 2016. 7(6): p. 915-918; Hughes, J., et al., Precipitation of Autoimmune Diabetes With Anti-PD-1 Immunotherapy. Diabetes Care, 2015. 38(4): p. e55; and Ansari, M. J., et al., The programmed death-1 (PD-1) pathway regulates autoimmune diabetes in nonobese diabetic (NOD) mice. J Exp Med, 2003. 198(1): p. 63-9) (and FIG. 2D); (3) αPD-1-ABD-PE-treated NOD mice resist the diabetes exacerbation effect of αPD-1, an inhibitor of the PD-1 immune checkpoint (FIG. 2D). These data suggest that αPD-1-ABD-PE abates those T1D-related, PD-1-positive cells in NOD mice. Thus, it is plausible to use αPD-1-ABD-PE to deplete T1D-related PD-1-positive cells in newly-diagnosed NOD mice and stop B-cell destruction, which may reverse T1D.

TABLE 1
Expected results.

| Study | Diabetes- | Islet | Lymphocyte | T cell | B cell |
|---|---|---|---|---|---|
| Treatment | free time | integrity | infiltration | fraction | fraction |
| αPD-1-ABD- | +++++ | High | + | Normal | Normal |
| PE | | | | | |
| ABD-PE | zero | na | na | Normal | Normal |
| αPD-1-PE | ++++ | na | na | Normal | Normal |
| αPD-1 | zero | na | na | Normal | Normal |
| αCD3 | ++++ | Medium | +++ | Low | Normal |
| PBS | zero | Low | +++++ | Normal | Normal |

Treatment design: As listed in Table 1, αPD-1-ABD-PE and ABD-PE will be compared: to prove that it is the PD-1-targeted depletion that leads to the reversal of T1D; and to demonstrate the effect of ABD. αPD-1-ABD-PE and αPD-1 scFv will be compared to show that αPD-1 alone does not reverse T1D (e.g., αPD-1 actually precipitates T1D, FIG. 2D). αPD-1-ABD-PE and aCD3 will be compared to evaluate the PD-1-targeted depletion versus the T lymphocyte depletion. PBS control will be used as a control. aCD3 will be generated from the hybridoma 145-2C11-γ3 (Penaranda, C., Q. Tang, and J. A. Bluestone, Anti-CD3 therapy promotes tolerance by selectively depleting pathogenic cells while preserving regulatory T cells. J Immunol, 2011. 187(4): p. 2015-22).

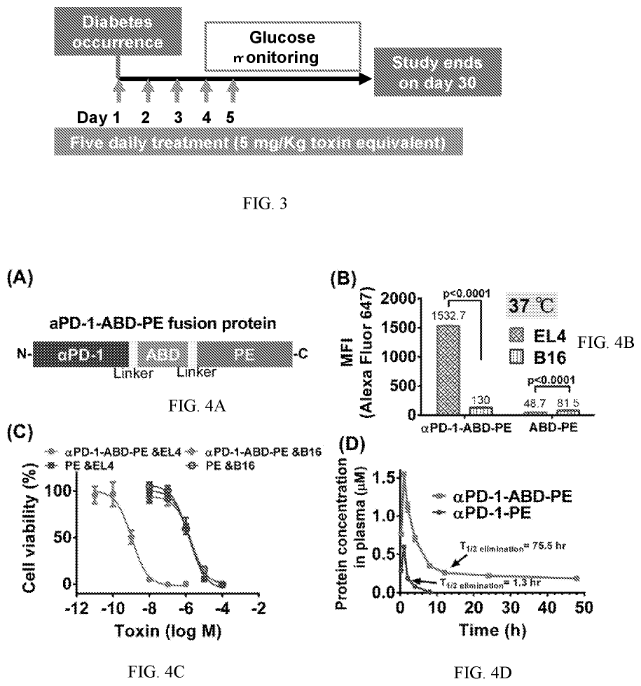

As shown in FIG. 3, each mouse will be begin treatment when its diabetes is first confirmed (e.g., its blood glucose concentration exceeds 250 mg/dL for two consecutive measurements). FIG. 3 also describes the dosing amount and schedule. Five doses are scheduled, however, the dosing numbers may increase after it is verified that αPD-1-ABD-PE does not abate B and T lymphocytes and has no obvious side effects. A T1D reversal is recognized when glucose concentrations of a mouse return to values smaller than 250 mg/dL for two consecutive measurements. Mice will be monitored for 30 days after the first treatment unless they are sacrificed at humane endpoints. The reversal effect will be quantified as diabetes-free survival time (Table 1).

The next set of experiments will evaluate the tissue integrity and the lymphocyte infiltration of pancreatic islets in newly-diagnosed T1D mice after the αPD-1-ABD-PE treatment. It is expected that the αPD-1-ABD-PE treatment will restore islet integrity and reduce lymphocyte infiltration. Progression of T1D are accompanied with the destruction of pancreatic islets and hyperlymphocyte infiltration in pancreases (In't Veld, P., Insulitis in human type 1 diabetes: The quest for an elusive lesion. Islets, 2011. 3(4): p. 131-138; and Chan, J., et al., Transplantation of bone marrow genetically engineered to express proinsulin II protects against autoimmune insulitis in NOD mice. J Gene Med, 2006. 8(11): p. 1281-90). Thus, if αPD-1-ABD-PE reverses T1D, these histological recoveries are expected. This study will be carried out once the reversal effect of αPD-1-ABD-PE is confirmed and the average time for the treated NOD mice to recover from T1D is determined. The treatments will include αPD-1-ABD-PE, aCD3 and PBS; the expected results of these treatments are listed in Table 1. The same treatment plan as shown in FIG. 3 will be used except that the pancreas from treated mice will be collected as a time point when half of the treated mice recover from T1D (e.g., as determined as described above). Histology analysis will be used to evaluate pancreatic islet integrity and lymphocyte infiltration (T and B cells) as previously described (Chan, J., et al., Transplantation of bone marrow genetically engineered to express proinsulin II protects against autoimmune insulitis in NOD mice. J Gene Med, 2006. 8(11): p. 1281-90; and Chatenoud, L., et al., Anti-CD3 antibody induces long-term remission of overt autoimmunity in nonobese diabetic mice. Proceedings of the National Academy of Sciences of the United States of America, 1994. 91(1): p. 123-127).

Quantification of T and B lymphocytes after the αPD-1-ABD-PE treatment will be performed. When blunt depletion (αCD-3 or αCD-20) was used, patients lost the majority of T or B lymphocytes for an extended period of time. In contrast, PD-1-targeted depletion is expected to selectively eliminate a small fraction of T and B lymphocytes, namely activated, PD-1-positive lymphocytes. It is expected that αPD-1-ABD-PE will not affect the systemic T and B lymphocyte (cell) fractions as compared to PBS (Table 1). In contrast, αCD-3 is expected to deplete T lymphocytes. These results support the notion that αPD-1-ABD-PE is safe to use and its usage will not compromise normal functions of the immune system.

The treatment schedule is listed in the Table 1. To conduct the study, 10-week old NOD mice will be treated using the same treatment plan as shown in FIG. 3 except that these mice will be sacrificed and their splenocytes will be collected immediately after the last dosing. The B and T lymphocyte fractions of the collected splenocytes will be quantified using flow cytometry using B and T lymphocyte markers.

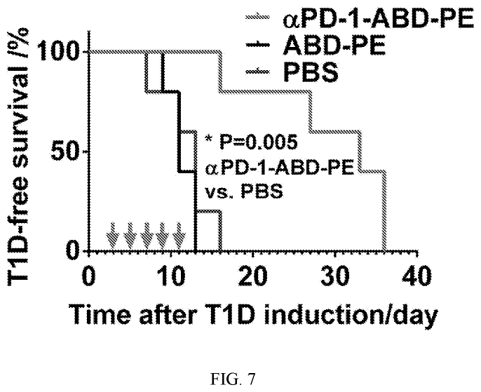

FIG. 7 shows that αPD-1-ABD-PE delays type 1 diabetes onset in cyclophosphamide-induced T1D mice. The induced mice were treated with αPD-1-ABD-PE (5 mg/kg for 5 times) or controls. The arrows point to the time of the treatments. The αPD-1-ABD-PE treated group showed significantly longer T1D-free survival (median, 33 days) than the two control groups (PBS, 13 days, p=0.0043; ABD-PE, 11 day, p=0.005; N=5).

### Example 5: αPD-ABD-PE Specifically Depletes PD-1-Positive Cells In Vitro and In Vivo

The first set of experiments were carried out to confirm that αPD-1-ABD-PE can be specifically internalized by PD-1-positive cells. To this end, the samples include Alexa647-labeled αPD-1-ABD-PE, ABD-PE, PE, and αPD-1. It is expected that (1) PD-1-positive cells will internalize significantly more αPD-1-ABD-PE than ABD-PE or PE and the same amount of αPD-1-ABD-PE and αPD-1; (2) PD-1-positive cells will internalize significantly more αPD-1-ABD-PE than PD-1-negative cells; and (3) PD-1-negative cells will internalize the same amount of the planned samples. The results in FIG. 4 also show that ABD-PE has low binding and uptake by both EL4 cells (PD-1-positive) than by B16 cells (PD-1-negative); and that PE is marginally toxic to both EL4 and B16 cells. The samples were injected intraperitoneally. The result demonstrates the importance of ABD in extending the half-life of αPD-1-ABD-PE.

For these experiments, the samples are incubated with PD-1-positive cells (EL4) and PD-1-negative cells (B16 melanoma cells) at 37° C. and 4° C. The cell-surface bound samples are measured after the 4° C. incubation and the sum of the surface bound and internalized samples after the 37° C. incubation as performed to generate data in FIG. 4B. The internalized sample amount will be inferred by comparing results of the 37° C. and 4° C. incubations.

The next set of experiments will be performed to confirm that αPD-1-ABD-PE is selectively toxic to PD-1-positive cells in vitro. To this end, the samples include αPD-1-ABD-PE, PE, and a mixture of ABD-PE and αPD-1. Based on the toxicity data in FIG. 4C, it is expected that (1) αPD-1-ABD-PE will be markedly more toxic to PD-1-positive cells than to PD-1-negative cells; (2) αPD-1-ABD-PE will be markedly more toxic to PD-1-positive cells as compared to PE or the mixture of ABD-PE and αPD-1; and (3) PE and the mixture will have equally low toxicity to both PD-1-positive and -negative cells. The mixture of ABD-PE and αPD-1 will serve as a control to prove that the PD-1-targeted toxicity and depletion require ABD-PE and αPD-1 to be linked together as one molecule.

The samples with EL4 cells and B16 cells will be incubated. Then, an MTS assay can be used to check cell viability as performed to generate data in FIG. 2C and as previously described (Zhao P, Xia G, Dong S, Jiang Z X, Chen M. An iTEP-salinomycin nanoparticle that specifically and effectively inhibits metastases of 4T1 orthotopic breast tumors. Biomaterials. 2016; 93:1-9. doi: 10.1016/j.biomaterials.2016.03.032. PubMed PMID: 27060212; PMCID: PMC4844807). Additionally, PD-1-positive cells will be induced from primary lymphocytes using a previously described (Agata Y, Kawasaki A, Nishimura H, Ishida Y, Tsubata T, Yagita H, Honjo T. Expression of the PD-1 antigen on the surface of stimulated mouse T and B lymphocytes. Int Immunol. 1996; 8(5):765-72. PubMed PMID: 8671665; Nishimura H, Agata Y, Kawasaki A, Sato M, Imamura S, Minato N, Yagita H, Nakano T, Honjo T. Developmentally regulated expression of the PD-1 protein on the surface of double-negative (CD4-CD8-) thymocytes. Int Immunol. 1996; 8(5):773-80. PubMed PMID: 8671666; and Yamazaki T, Akiba H, Iwai H, Matsuda H, Aoki M, Tanno Y, Shin T, Tsuchiya H, Pardoll D M, Okumura K, Azuma M, Yagita H. Expression of programmed death 1 ligands by murine T cells and APC. Journal of immunology (Baltimore, Md.: 1950), 2002; 169(10):5538-45. PubMed PMID: 12421930) so that primary lymphocytes can be used.

Experiments that examine whether αPD-1-ABD-PE specifically depletes PD-1-positive cells in vivo will be performed. The samples include αPD-1-ABD-PE, PBS, and a mixture of ABD-PE and αPD-1. Based on the depletion data shown in FIGS. 5A and 5B, it is expect that (1) αPD-1-ABD-PE will deplete PD-1-positive cells but not general B and T cell fractions (the majority of B and T cells are naïve, PD-1-negative); and (2) PBS and the mixture will not deplete any of the above cell populations.

To this end, EL4 cells will be transferred into mice because endogenous PD-1 positive cells are scarce in the body (Agata Y, Kawasaki A, Nishimura H, Ishida Y, Tsubata T, Yagita H, Honjo T. Expression of the PD-1 antigen on the surface of stimulated mouse T and B lymphocytes. Int Immunol. 1996; 8(5):765-72. PubMed PMID: 8671665). Then the fractions of EL4, B cells, CD4 T cells, and CD8 T cells will be quantified among lymphocytes. Last, the cell fractions of the αPD-1-ABD-PE-treated group and the control groups will be compared. FIG. 5A shows that the EL4 fraction of lymphocytes in the toxin-treated mice was 1.05%, 10 times smaller than that fraction in the PBS-treated mice, suggesting the toxin depletes PD-1-positive cells in vivo.

Next, the impact of αPD-1-ABD-PE on antibody and CTL responses in healthy mice will be evaluated. The samples include αPD-1-ABD-PE, PBS, and αCD-3. aCD3 will serve as a control because it (clone 145-2c11) is commercially available and compromises T cell-mediated responses (Hirsch R, Eckhaus M, Auchincloss H, Jr., Sachs D H, Bluestone J A. Effects of in vivo administration of anti-T3 monoclonal antibody on T cell function in mice. I. Immunosuppression of transplantation responses. Journal of immunology (Baltimore, Md.: 1950). 1988; 140(11):3766-72. PubMed PMID: 3286764). Based on the data in FIGS. 5B and 5C and the above information on aCD3, it is expected that (1) the αPD-1-ABD-PE-treated mice will mount normal antibody and CTL responses just as the PBS-treated mice; (2) the αCD-3-treated mice will have impaired CTL responses; and (d) the mice will have normal antibody responses because DNP-Ficoll, a T-cell-independent model antigen, will be used in these experiments. The results shown in FIG. 5B suggest that the mice treated with fusion protein (e.g., αPD-1-ABD-PE) were not depleted of their bulk B and T cells. In contrast, aCD3 depleted both CD4- and CD8-positive T cells. e.g. the CD4-positive fraction in the aCD3-treated mice was 1/80 of that in the αPD-1-ABD-PE-treated mice. FIG. 5C shows that PD-1-targeted depletion preserves B cell immunity. This result distinguishes the depletion from DMTs, like alematuzumab, that compromise adaptive immunity for months (Wingerchuk D M, Carter J L. Multiple sclerosis: current and emerging disease-modifying therapies and treatment strategies. Mayo Clin Proc. 2014; 89(2):225-40. doi: 10.1016/j.mayocp.2013.11.002. PubMed PMID: 24485135; and Turner M J, Lamorte M J, Chretien N, Havari E, Roberts B L, Kaplan J M, Siders W M. Immune status following alemtuzumab treatment in human CD52 transgenic mice. J Neuroimmunol. 2013; 261(1-2):29-36)

C57B/L6 mice will be treated with αPD-1-ABD-PE or a controls (see, FIG. 5C). Then, each treatment group will be separated into two sub-groups. One sub-group will be vaccinated with DNP-Ficoll as previously described (Turner M J, Lamorte M J, Chretien N, Havari E, Roberts B L, Kaplan J M, Siders W M. Immune status following alemtuzumab treatment in human CD52 transgenic mice. J Neuroimmunol. 2013; 261(1-2):29-36) and anti-DNP antibody titers will be evaluated using ELISA (Turner M J, Lamorte M J, Chretien N, Havari E, Roberts B L, Kaplan J M, Siders W M. Immune status following alemtuzumab treatment in human CD52 transgenic mice. J Neuroimmunol. 2013; 261(1-2):29-36) (see, FIG. 5C). the second sub-group will be vaccinated with a mixture of CTL epitopes (SIINFEKL (SEQ ID NO: 7) and SVYDFFVWL (SEQ ID NO: 8) in IFA. These epitopes can be used to elicit CTL responses (Dong S, Xu T, Zhao P, Parent K N, Chen M. A Comparison Study of iTEP Nanoparticle-Based CTL Vaccine Carriers Revealed a Surprise Relationship between the Stability and Efficiency of the Carriers. Theranostics. 2016; 6(5):666-78). The CTL responses will be quantified (Dong S, Xu T, Zhao P, Parent K N, Chen M. A Comparison Study of iTEP Nanoparticle-Based CTL Vaccine Carriers Revealed a Surprise Relationship between the Stability and Efficiency of the Carriers. Theranostics. 2016; 6(5):666-78).

### Example 6: PD-1-Targeted Depletion Stops Autoimmune Attacks in EAE without Undermining Adaptive Immunity

Study design: First, autoimmune attacks will be evaluated in EAE through two scores, the clinical score and the CNS pathology score (Tsunoda I, Tanaka T, Terry E J, Fujinami R S. Contrasting roles for axonal degeneration in an autoimmune versus viral model of multiple sclerosis: When can axonal injury be beneficial? Am J Pathol. 2007; 170(1):214-26). These two scores are commonly used to evaluate EAE progression and the efficacy of DMTs (Farooqi N, Gran B, Constantinescu C S. Are current disease-modifying therapeutics in multiple sclerosis justified on the basis of studies in experimental autoimmune encephalomyelitis? J Neurochem. 2010; 115(4):829-44). A reduction of autoimmune attacks can be inferred from a decrease of the two scores. The infiltration of PD-1-positive cells in spinal cords will also be examined because it is possible that PD-1-targeted depletion may decrease the infiltration and, in turn, reduce autoimmune attacks. Second, the adaptive immunity of mice that are induced with EAE and receive PD-1-targeted depletion will be examined by assessing autoimmune attacks and adaptive immunity. Last, two EAE models will be used: a chronic, non-remitting model (C57BL/6 mice immunized with the MOG₃₅₋₅₅ peptide from myelin oligodendrocyte glycoprotein) and a relapsing model (SJL/J mice immunized with the PLP₁₃₉₋₁₅₁ peptide from proteolipid protein) (Rangachari M, Kuchroo V K. Using EAE to better understand principles of immune function and autoimmune pathology. J Autoimmun. 2013; 45:31-9). These models are used because efficacy results that were obtained from these models are well reproducible in clinical studies (Farooqi N, Gran B, Constantinescu C S. Are current disease-modifying therapeutics in multiple sclerosis justified on the basis of studies in experimental autoimmune encephalomyelitis? J Neurochem. 2010; 115(4):829-44; and Constantinescu C S, Farooqi N, O'Brien K, Gran B. Experimental autoimmune encephalomyelitis (EAE) as a model for multiple sclerosis (MS). Br J Pharmacol. 2011; 164(4):1079-106), and these models simulate two distinct courses of MS. Thus, if PD-1-targeted depletion is proven effective in the both models, it is reasonable to expect that the depletion is effective in two different courses of MS. Each EAE model will be used to evaluate autoimmune attack and adaptive immunity.

The first set of experiments will evaluate whether PD-1-targeted depletion stops autoimmune attack in a chronic EAE model. MOG₃₅₋₅₅ induces chronic EAE. PD-1-positive cells play a role in EAE progression of this model (Liang S C, Latchman Y E, Buhlmann J E, Tomczak M F, Horwitz B H, Freeman G J, Sharpe A H. Regulation of PD-1, P D-L1, and P D-L2 expression during normal and autoimmune responses. Eur J Immunol. 2003; 33(10):2706-16. doi: 10.1002/eji.200324228. PubMed PMID: 14515254; Zhu B, Guleria I, Khosroshahi A, Chitnis T, Imitola J, Azuma M, Yagita H, Sayegh M H, Khoury S J. Differential role of programmed death-ligand 1 [corrected] and programmed death-ligand 2 [corrected] in regulating the susceptibility and chronic progression of experimental autoimmune encephalomyelitis. Journal of immunology (Baltimore, Md.: 1950). 2006; 176(6):3480-9; Salama A D, Chitnis T, Imitola J, Ansari M J, Akiba H, Tushima F, Azuma M, Yagita H, Sayegh M H, Khoury S J. Critical role of the programmed death-1 (PD-1) pathway in regulation of experimental autoimmune encephalomyelitis. The Journal of experimental medicine. 2003; 198(1):71-8; and Schreiner B, Bailey S L, Shin T, Chen L, Miller S D. PD-1 ligands expressed on myeloid-derived APC in the CNS regulate T-cell responses in EAE. Eur J Immunol. 2008; 38(10):2706-17). Mice will have an elevated infiltration of PD-1-positive cells in their spinal cords when their EAE progresses (Liang S C, Latchman Y E, Buhlmann J E, Tomczak M F, Horwitz B H, Freeman G J, Sharpe A H. Regulation of PD-1, PD-L1, and PD-L2 expression during normal and autoimmune responses. Eur J Immunol. 2003; 33(10):2706-16; and Salama A D, Chitnis T, Imitola J, Ansari M J, Akiba H, Tushima F, Azuma M, Yagita H, Sayegh M H, Khoury S J. Critical role of the programmed death-1 (PD-1) pathway in regulation of experimental autoimmune encephalomyelitis. The Journal of experimental medicine. 2003; 198(1):71-8). PD-1-targeted depletion exerted by αPD-1-ABD-PE may decrease the clinical and pathology scores of the mice and reduce the infiltration of PD-1-positive cells.

TABLE 2
Treatments and expected scores

| | Task 1 (chronic) | | Tasks 3 (relapsing) | |
|---|---|---|---|---|
| | Clinical | Pathology | Clinical | Pathology |
| Treatment | score | score | score | score |
| αPD-1-ABD-PE | 0-1 | <20% | 0-1 | <20% |
| Mixture of | ~3 | >50% | ~3 | >50% |
| ABD-PE and | | | | |
| αPD-1 | | | | |
| PBS | ~3 | ~50% | ~3 | >50% |

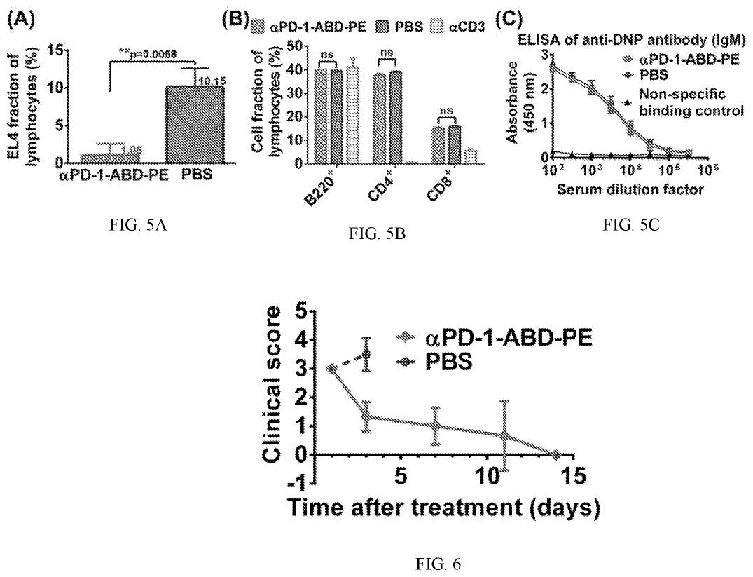

The samples are listed in Table 2. Based on FIG. 6, it is expected that (1) αPD-1-ABD-PE will decrease the clinical and pathology scores and reduce the infiltration of PD-1-positive cells as compared to PBS; (2) the mixture of αPD-1 and ABD-PE will have the same effect as PBS. Although αPD-1, in theory, promotes autoimmunity exerted by PD-1-positive cells (Salama A D, Chitnis T, Imitola J, Ansari M J, Akiba H, Tushima F, Azuma M, Yagita H, Sayegh M H, Khoury S J. Critical role of the programmed death-1 (PD-1) pathway in regulation of experimental autoimmune encephalomyelitis. The Journal of experimental medicine. 2003; 198(1):71-8), the αPD-1 dose used in this study (5 mg/Kg BW αPD-1-ABD-PE equivalent) is too low for an effect. ABD-PE will not impact EAE progression. Table 1 summarizes the expected clinical and histology scores. FIG. 6 shows that the clinical score of these mice dropped from 3 to 0. The efficacy of the toxin is compared favorably to that of existing DMTs because exiting DMTs have not been effective to the score 3 EAE (heien B E, Vanderlugt C L, Nickerson-Nutter C, Cornebise M, Scott D M, Perper S J, Whalley E T, Miller S D. Differential effects of treatment with a small-molecule VLA-4 antagonist before and after onset of relapsing EAE. Blood. 2003; 102(13):4464-71; and Myers K J, Witchell D R, Graham M J, Koo S, Butler M, Condon T P. Antisense oligonucleotide blockade of alpha 4 integrin prevents and reverses clinical symptoms in murine experimental autoimmune encephalomyelitis. J Neuroimmunol. 2005; 160(1-2):12-24). The majority of the PBS-treated mice had to be euthanized at day 3 due to their worsening morbidity (n=6).

The experimental procedures used are based on study described herein and two studies that used the same model to evaluate the efficacy of a DMT, glatiramer acetate (Begum-Haque S, Sharma A, Kasper I R, Foureau D M, Mielcarz D W, Haque A, Kasper L H. Downregulation of IL-17 and IL-6 in the central nervous system by glatiramer acetate in experimental autoimmune encephalomyelitis. J Neuroimmunol. 2008; 204(1-2):58-65; and Aharoni R, Arnon R, Eilam R. Neurogenesis and neuroprotection induced by peripheral immunomodulatory treatment of experimental autoimmune encephalomyelitis. J Neurosci. 2005; 25(36):8217-28).

EAE induction: 0.2 mg of CFA adjuvant-emulsified MOG₃₅₋₅₅ (MEVGWYRSPFSRVVHLYRNGK (SEQ ID NO: 9)) per mouse with pertussis toxin will be injected.

Depletion and control treatment: Induced mice will be randomly assigned into 3 groups. Mice will be evaluated and a clinical score will be recorded twice a week. When the score of any mouse reaches 3, this mouse will be treated with a single dose of αPD-1-ABD-PE or one of the two control treatments in Table 2 depending on the assignment of the mouse.

Assessment of clinical scores: The clinical score assessment will be continued until 30 days after the depletion or the control treatments. The mean scores of different treatments at multiple time points (Libbey J E, Doty D J, Sim J T, Cusick M F, Round J L, Fujinami R S. The effects of diet on the severity of central nervous system disease: One part of lab-to-lab variability. Nutrition. 2016; 32(7-8):877-83; and Miller S D, Karpus W J. Experimental autoimmune encephalomyelitis in the mouse. Curr Protoc Immunol. 2007; Chapter 15:Unit 15 1) will be compared.

Assessment of pathology scores: A separate cohort of mice will be used and they will be treated as described herein. At 30 days after the treatments, pathology scores of these mice will be obtained by integrating three sub-scores: the demyelination, perivascular cuff, meningitis scores, using a previously described (Libbey J E, Doty D J, Sim J T, Cusick M F, Round J L, Fujinami R S. The effects of diet on the severity of central nervous system disease: One part of lab-to-lab variability. Nutrition. 2016; 32(7-8):877-83). Different treatments will be compared by their mean pathology scores.

Assessment of the PD-1-positive cell infiltration: The infiltrations will be morphologically compared between different treatments using samples from procedure described herein.

Next, the impact of αPD-1-ABD-PE on adaptive immunity in a chronic EAE model will be evaluated. The impact of PD-1-targeted depletion on adaptive immunity will be evaluated in those mice that have EAE. This evaluation design is clinically relevant because the depletion will be applied to MS patients. Based on the data in FIGS. 5B and 5C, it is expected that (1) the αPD-1-ABD-PE-treated mice will mount normal antibody and CTL responses to vaccines just as the PBS-treated mice; and (2) the mixture of αPD-1 and ABD-PE will not affect antibody and CTL responses. EAE will be induced and mice will be treated as described herein. Then, antibody and CTL responses in these mice will be induced and examined.

The following set of experiments will be carried out to evaluate whether PD-1-targeted depletion stops an autoimmune attack in a relapsing EAE model. PLP₁₃₉₋₁₅₁ induces relapsing EAE. PD-1-positive cells promote EAE progression (Zhu B, Guleria I, Khosroshahi A, Chitnis T, Imitola J, Azuma M, Yagita H, Sayegh M H, Khoury S J. Differential role of programmed death-ligand 1 [corrected] and programmed death-ligand 2 [corrected] in regulating the susceptibility and chronic progression of experimental autoimmune encephalomyelitis. Journal of immunology (Baltimore, Md.: 1950). 2006; 176(6):3480-9; and Schreiner B, Bailey S L, Shin T, Chen L, Miller S D. PD-1 ligands expressed on myeloid-derived APC in the CNS regulate T-cell responses in EAE. Eur J Immunol. 2008; 38(10):2706-17). The depletion may decrease clinical and pathology scores in the mice previously EAE-induced. The depletion may also reduce the infiltration of PD-1-positive cells into the spinal cords of these mice. The samples are listed in Table 2. Based on the above premises (Baltimore, Md.: 1950). 2006; 176(6):3480-9; and Schreiner B, Bailey S L, Shin T, Chen L, Miller S D. PD-1 ligands expressed on myeloid-derived APC in the CNS regulate T-cell responses in EAE. Eur J Immunol. 2008; 38(10):2706-17), it is expected that (1) αPD-1-ABD-PE will decrease clinical and pathology scores and reduce the infiltration of PD-1-positive cells as compared to PBS; and (2) the mixture of αPD-1 and ABD-PE will have no effect.

These experimental procedures are based on two studies that use the same model to evaluate the efficacy of a DMT, natalizumab (Theien B E, Vanderlugt C L, Nickerson-Nutter C, Cornebise M, Scott D M, Perper S J, Whalley E T, Miller S D. Differential effects of treatment with a small-molecule VLA-4 antagonist before and after onset of relapsing EAE. Blood. 2003; 102(13):4464-71; and Myers K J, Witchell D R, Graham M J, Koo S, Butler M, Condon T P). Antisense oligonucleotide blockade of alpha 4 integrin prevents and reverses clinical symptoms in murine experimental autoimmune encephalomyelitis. J Neuroimmunol. 2005; 160(1-2):12-24) EAE will be by injecting 0.04 mg of CFA-emulsified PLP₁₃₉₋₁₅₁ (HSLGKWLGHPDKF (SEQ ID NO: 10)) per mouse with pertussis toxin. These induced mice will randomly placed into 3 groups for 3 treatments as shown in Table 2. Any induced mouse will be treated once her clinical score reaches 3. The scoring procedures and the assessment of PD-1-positive cell infiltration will be the same as disclosed herein.

Next, the impact of αPD-1-ABD-PE on adaptive immunity in a relapsing EAE model will be evaluated. CD4 T cell responses will be checked instead of CTL responses as a part of the adaptive immunity assessment because there are established epitopes and immunization protocols to induce CD4 T cell responses in SJL/J mice (Zamvil S S, Mitchell D J, Powell M B, Sakai K, Rothbard J B, Steinman L. Multiple discrete encephalitogenic epitopes of the autoantigen myelin basic protein include a determinant for I-E class II-restricted T cells. The Journal of experimental medicine. 1988; 168(3):1181-6; Sakai K, Zamvil S S, Mitchell D J, Lim M, Rothbard J B, Steinman L. Characterization of a major encephalitogenic T cell epitope in SJL/J mice with synthetic oligopeptides of myelin basic protein. J Neuroimmunol. 1988; 19(1-2):21-32; and Sakai K, Sinha A A, Mitchell D J, Zamvil S S, Rothbard J B, McDevitt H O, Steinman L. Involvement of distinct murine T-cell receptors in the autoimmune encephalitogenic response to nested epitopes of myelin basic protein. Proceedings of the National Academy of Sciences of the United States of America. 1988; 85(22):8608-12). Both CD 4 T cell responses and CTL responses represent the T cellular arm of adaptive immunity. Based on the preliminary data in FIGS. 5B and 5C, it is expected that (1) the αPD-1-ABD-PE-treated mice will mount normal antibody and CD4 T cell responses just as the PBS-treated mice; and (2) the mixture of αPD-1 and ABD-PE will not affect antibody and CD4 T cell responses.

EAE-induced mice will be treat as described herein. For antibody responses, the evaluation will be as described herein. For CD4 T cell responses, mice will be vaccinated with an epitope mixture, VHFFKNIVPRTP (SEQ ID NO: 11) and TGILDSIGRFFSG (SEQ ID NO: 12), in IFA as previously described (Zamvil S S, Mitchell D J, Powell M B, Sakai K, Rothbard J B, Steinman L. Multiple discrete encephalitogenic epitopes of the autoantigen myelin basic protein include a determinant for I-E class II-restricted T cells. The Journal of experimental medicine. 1988; 168(3):1181-6; Sakai K, Zamvil S S, Mitchell D J, Lim M, Rothbard J B, Steinman L. Characterization of a major encephalitogenic T cell epitope in SJL/J mice with synthetic oligopeptides of myelin basic protein. J Neuroimmunol. 1988; 19(1-2):21-32; and Sakai K, Sinha A A, Mitchell D J, Zamvil S S, Rothbard J B, McDevitt H O, Steinman L. Involvement of distinct murine T-cell receptors in the autoimmune encephalitogenic response to nested epitopes of myelin basic protein. Proceedings of the National Academy of Sciences of the United States of America. 1988; 85(22):8608-12). CD4 T cell responses will be quantified by an ELISPOT assay (Dong S, Xu T, Zhao P, Parent K N, Chen M. A Comparison Study of iTEP Nanoparticle-Based CTL Vaccine Carriers Revealed a Surprise Relationship between the Stability and Efficiency of the Carriers. Theranostics. 2016; 6(5):666-78).

Statistical test: An equivalence, two one-sided test with a significance level of 0.05 will be used (Walker E, Nowacki A S. Understanding equivalence and noninferiority testing. J Gen Intern Med. 2011; 26(2):192-6. doi: 10.1007/s11606-010-1513-8); the hypothesis is that αPD-1-ABD-PE is not statistically different from PBS. A non-equivalence, two-sample t-tests (or their nonparametric alternative) with a significance level of 0.05 will be used to examine if αPD-1-ABD-PE is different from its controls in these analyses. Power analysis: pPower analyses will be performed based on preliminary data, published data, and an assumption of a normal distribution of the data. The analyses will be performed with 80% power and a significance level of 0.05. If the data are not normally distributed, a nonparametric test will be performed and 15% more mice will be used to meet the group size requirement of a nonparametric test.

### Example 7: Depletion of Programmed Death-1 Positive Cells Leads to Specific Suppression of Autoimmune Diseases

Targeted suppression of autoimmune diseases without collateral suppression of normal immunity remains an elusive but a clinically important goal. The data described herein shows that targeted depletion of a subset of immune cells, programmed death-1 receptor positive cells (PD-1⁺ cells), markedly and specifically suppresses autoimmune diseases. To facilitate this depletion, an immunotoxin consisting of an anti-PD-1 scFv (αPD-1), an albumin-binding domain (ABD), and a Pseudomonas exotoxin (PE) was engineered. This immunotoxin, termed αPD-1-ABD-PE, selectively binds to PD-1⁺ cells and is hundreds of times more lethal for PD-1⁺ cells than for PD-1-negative (PD-1⁻) cells. Significantly, αPD-1-ABD-PE administration delays the onset of autoimmune diabetes and allows full recovery of clinical disease of mice paralyzed with experimental autoimmune encephalomyelitis (EAE). The αPD-1-ABD-PE treatment also reduced PD-1⁺ cells, total T cells, cells of an autoreactive T cell clone in inflamed organs of the two models, autoimmune diabetes and EAE. Equally important, αPD-1-ABD-PE treated mice maintained normal adaptive immunity, evidenced by their full-strength immune responses to vaccinations. Thus, depletion of PD-1⁺ cells provides a targeted approach to halt autoimmune diseases while preserving healthy adaptive immunity.

Introduction.

Autoimmune diseases are primarily mediated by auto-reactive lymphocytes and/or their secreted auto-antibodies (Bluestone, J. A., Herold, K. & Eisenbarth, G. Genetics, pathogenesis and clinical interventions in type 1 diabetes. Nature 464, 1293-1300 (2010); Atkinson, M. A., Eisenbarth, G. S. & Michels, A. W. Type 1 diabetes. Lancet 383, 69-82 (2014); Laurence, A. & Aringer, M. in The Autoimmune Diseases, Edn. 5th. (ed. N. R. M. Rose, I. R.) 311-318 (Elsevier, San Diego; 2014); and van Belle, T. L., Coppieters, K. T. & von Herrath, M. G. Type 1 diabetes: etiology, immunology, and therapeutic strategies. Physiol Rev 91, 79-118 (2011)). Targeted suppression of certain lymphocyte populations is an effective strategy to treat the diseases that has yielded new therapies for multiple sclerosis (MS) and systemic lupus erythematosus (SLE) (Farber, R., Harel, A. & Lublin, F. Novel Agents for Relapsing Forms of Multiple Sclerosis. Annu Rev Med 67, 309-321 (2016); Wingerchuk, D. M. & Carter, J. L. Multiple sclerosis: current and emerging disease-modifying therapies and treatment strategies. Mayo Clin Proc 89, 225-240 (2014); Kim, S. S., Kirou, K. A. & Erkan, D. Belimumab in systemic lupus erythematosus: an update for clinicians. Therapeutic Advances in Chronic Disease 3, 11-23 (2012); and Chatenoud, L. in The autoimmune diseases, Edn. 4th. (ed. N. R. M. Rose, I. R.) 1121-1145 (Elesvier, San Diego, Calif., USA; 2014)). However, these therapies are rarely considered as first-line therapeutic options due to their indiscriminate inhibition of normal adaptive immunity (Wingerchuk, D. M. & Carter, J. L. Multiple sclerosis: current and emerging disease-modifying therapies and treatment strategies. Mayo Clin Proc 89, 225-240 (2014); Elsegeiny, W., Eddens, T., Chen, K. & Kolls, J. K. Anti-CD20 antibody therapy and susceptibility to Pneumocystis pneumonia. Infection and immunity 83, 2043-2052 (2015), Genzyme (Cambridge, Mass.; 2014); Torkildsen, Ø., Myhr, K. M. & Bø, L. Disease-modifying treatments for multiple sclerosis—a review of approved medications. European Journal of Neurology 23, 18-27 (2016); and McNamara, C., Sugrue, G., Murray, B. & MacMahon, P. J. Current and Emerging Therapies in Multiple Sclerosis: Implications for the Radiologist, Part 2-Surveillance for Treatment Complications and Disease Progression. AJNR Am J Neuroradiol (2017)). This inhibition occurs because these therapies target lymphocytes too broadly (van Belle, T. L., Coppieters, K. T. & von Herrath, M. G. Type 1 diabetes: etiology, immunology, and therapeutic strategies. Physiol Rev 91, 79-118 (2011); Farber, R., Harel, A. & Lublin, F. Novel Agents for Relapsing Forms of Multiple Sclerosis. Annu Rev Med 67, 309-321 (2016); Wingerchuk, D. M. & Carter, J. L. Multiple sclerosis: current and emerging disease-modifying therapies and treatment strategies. Mayo Clin Proc 89, 225-240 (2014); and Torkildsen, Ø., Myhr, K. M. & Bø, L. Disease-modifying treatments for multiple sclerosis—a review of approved medications. European Journal of Neurology 23, 18-27 (2016)). Thus, identification and selective suppression of pathogenic lymphocytes responsible for autoimmune diseases while keeping non-pathologic lymphocytes intact constitute an overarching yet unmet clinical goal.

PD-1⁺ cells are primarily activated B and T cells or B and T effector cells (Francisco, L. M., Sage, P. T. & Sharpe, A. H. The PD-1 Pathway in Tolerance and Autoimmunity. Immunological reviews 236, 219-242 (2010); Agata, Y. et al. Expression of the PD-1 antigen on the surface of stimulated mouse T and B lymphocytes. Int Immunol 8, 765-772 (1996); and Yamazaki, T. et al. Expression of programmed death 1 ligands by murine T cells and APC. J Immunol 169, 5538-5545 (2002)). PD-1, a negative receptor on these cells, switches on the PD-1 immune checkpoint when engaged by its ligands. This checkpoint counteracts immune stimulatory signals and limits PD-1⁺ effector cells from initiating autoimmune destruction (Joller, N., Peters, A., Anderson, A. C. & Kuchroo, V. K. Immune checkpoints in central nervous system autoimmunity. Immunol Rev 248, 122-139 (2012); Liang, S. C. et al. Regulation of PD-1, PD-L1, and PD-L2 expression during normal and autoimmune responses. Eur J Immunol 33, 2706-2716 (2003); Zhu, B. et al. Differential role of programmed death-ligand 1 [corrected] and programmed death-ligand 2 [corrected] in regulating the susceptibility and chronic progression of experimental autoimmune encephalomyelitis. J Immunol 176, 3480-3489 (2006); Salama, A. D. et al. Critical role of the programmed death-1 (PD-1) pathway in regulation of experimental autoimmune encephalomyelitis. J Exp Med 198, 71-78 (2003); Latchman, Y. E. et al. PD-L1-deficient mice show that PD-L1 on T cells, antigen-presenting cells, and host tissues negatively regulates T cells. Proc Natl Acad Sci USA 101, 10691-10696 (2004); Okazaki, T., Chikuma, S., Iwai, Y., Fagarasan, S. & Honjo, T. A rheostat for immune responses: the unique properties of PD-1 and their advantages for clinical application. Nat Immunol 14, 1212-1218 (2013); Keir, M. E., Butte, M. J., Freeman, G. J. & Sharpe, A. H. PD-1 and its ligands in tolerance and immunity. Annu Rev Immunol 26, 677-704 (2008); and Fife, B. T. et al. Interactions between PD-1 and PD-L1 promote tolerance by blocking the TCR-induced stop signal. Nat Immunol 10, 1185-1192 (2009). However, in type 1 diabetes (T1D), MS, SLE, and rheumatoid arthritis, the PD-1 checkpoint fails to stop autoimmune destruction (Joller, N., Peters, A., Anderson, A. C. & Kuchroo, V. K. Immune checkpoints in central nervous system autoimmunity. Immunol Rev 248, 122-139 (2012); and Okazaki, T., Chikuma, S., Iwai, Y., Fagarasan, S. & Honjo, T. A rheostat for immune responses: the unique properties of PD-1 and their advantages for clinical application. Nat Immunol 14, 1212-1218 (2013)). Instead, PD-1⁺ cells infiltrate tissues (Liang, S. C. et al. Regulation of PD-1, PD-L1, and PD-L2 expression during normal and autoimmune responses. Eur J Immunol 33, 2706-2716 (2003); and Salama, A. D. et al. Critical role of the programmed death-1 (PD-1) pathway in regulation of experimental autoimmune encephalomyelitis. J Exp Med 198, 71-78 (2003)), and this infiltration escalates as the autoimmune diseases progress (Salama, A. D. et al. Critical role of the programmed death-1 (PD-1) pathway in regulation of experimental autoimmune encephalomyelitis. J Exp Med 198, 71-78 (2003)). These observations indicate that PD-1⁺ cells are important mediators of autoimmune diseases. Consistent with this concept, the blockade of the PD-1 checkpoint, which leads to a proliferation of PD-1⁺ cells, exacerbates autoimmune diseases in both human and mouse models (Salama, A. D. et al. Critical role of the programmed death-1 (PD-1) pathway in regulation of experimental autoimmune encephalomyelitis. J Exp Med 198, 71-78 (2003); Godwin, J. L. et al. Nivolumab-induced autoimmune diabetes mellitus presenting as diabetic ketoacidosis in a patient with metastatic lung cancer. Journal for immunotherapy of cancer 5, 40 (2017); Ansari, M. J. et al. The programmed death-1 (PD-1) pathway regulates autoimmune diabetes in nonobese diabetic (NOD) mice. J Exp Med 198, 63-69 (2003); and Hughes, J. et al. Precipitation of autoimmune diabetes with anti-PD-1 immunotherapy. Diabetes Care 38, e55-57 (2015)). Taken together, the targeted depletion of PD-1⁺ cells (or PD-1⁺ cell depletion) in the context of autoimmune diseases might be an effective method to assuage autoimmunity. It is worth mentioning that PD-1⁺ cell depletion is a different concept than ablation of the PD-1-gene. PD-1⁺ cell depletion eliminates activated lymphocytes; in contrast, the knockout of the PD-1 gene leaves activated lymphocytes without control by the PD-1 checkpoint and allows for uncontrolled proliferation of activated lymphocytes (Okazaki, T., Chikuma, S., Iwai, Y., Fagarasan, S. & Honjo, T. A rheostat for immune responses: the unique properties of PD-1 and their advantages for clinical application. Nat Immunol 14, 1212-1218 (2013)). Thus, PD-1⁺ cell depletion is expected to suppress autoimmunity while the knockout predisposes the host to enhanced autoimmunity.

There are two advantages for using PD-1⁺ cell depletion. First, the depletion should leave naïve lymphocytes (PD-1⁻) intact and hence preserve B and T cell repertoires because the depletion primarily applies to activated lymphocytes. PD-1⁺ cell depletion should not significantly compromise normal adaptive immunity, which distinguishes the depletion from current drugs used to treat autoimmune diseases such as natalizumab and alematuzumab (Farber, R., Harel, A. & Lublin, F. Novel Agents for Relapsing Forms of Multiple Sclerosis. Annu Rev Med 67, 309-321 (2016); Wingerchuk, D. M. & Carter, J. L. Multiple sclerosis: current and emerging disease-modifying therapies and treatment strategies. Mayo Clin Proc 89, 225-240 (2014); and Torkildsen, Ø., Myhr, K. M. & Bø, L. Disease-modifying treatments for multiple sclerosis—a review of approved medications. European Journal of Neurology 23, 18-27 (2016)). Second, PD-1⁺ cell depletion applies to both activated B and activated T cells since the both cells are PD-1-positive. The dual coverage of both activated B and T cells is advantageous because the both cells can contribute to autoimmune diseases (Laurence, A. & Aringer, M. in The Autoimmune Diseases, Edn. 5th. (ed. N. R. M. Rose, I. R.) 311-318 (Elsevier, San Diego; 2014)).

As described herein, αPD-1-ABD-PE can serve as a tool for PD-1⁺ cell depletion. This immunotoxin consists of a single-chain variable fragment (scFv) of αPD-1 (Zhao, P. et al. An Anti-Programmed Death-1 Antibody (αPD-1) Fusion Protein That Self-Assembles into a Multivalent and Functional αPD-1 Nanoparticle. Molecular pharmaceutics 14, 1494-1500 (2017)), an albumin-binding domain (ABD) Levy, O. E. et al. Novel exenatide analogs with peptidic albumin binding domains: potent anti-diabetic agents with extended duration of action. PLoS One 9, e87704 (2014); and Wang, P. et al. An albumin binding polypeptide both targets cytotoxic T lymphocyte vaccines to lymph nodes and boosts vaccine presentation by dendritic cells. Theranostics (In press), and a Pseudomonas exotoxin (PE) (Weldon, J. E. et al. A recombinant immunotoxin against the tumor-associated antigen mesothelin reengineered for high activity, low off-target toxicity, and reduced antigenicity. Mol Cancer Ther 12, 48-57 (2013); and Onda, M. et al. Recombinant immunotoxin against B-cell malignancies with no immunogenicity in mice by removal of B-cell epitopes. Proc Natl Acad Sci USA 108, 5742-5747 (2011)). The αPD-1 scFv serves as a targeting moiety. The ABD is used to extend plasma presence of αPD-1-ABD-PE because ABD-containing molecules have long plasma half-lives (Levy, O. E. et al. Novel exenatide analogs with peptidic albumin binding domains: potent anti-diabetic agents with extended duration of action. PLoS One 9, e87704 (2014); Wang, P. et al. An albumin binding polypeptide both targets cytotoxic T lymphocyte vaccines to lymph nodes and boosts vaccine presentation by dendritic cells. Theranostics (In press); and Chen, H. et al. Chemical Conjugation of Evans Blue Derivative: A Strategy to Develop Long-Acting Therapeutics through Albumin Binding. Theranostics 6, 243-253 (2016)). The PE has demonstrated to have clinical efficacy and is safe (Weldon, J. E. et al. A recombinant immunotoxin against the tumor-associated antigen mesothelin reengineered for high activity, low off-target toxicity, and reduced antigenicity. Mol Cancer Ther 12, 48-57 (2013); and Hassan, R., Alewine, C. & Pastan, I. New Life for Immunotoxin Cancer Therapy. Clin Cancer Res 22, 1055-1058 (2016)). αPD-1-ABD-PE possesses selective toxicity, both in vitro and in vivo, to PD-1⁺ cells. More importantly, αPD-1-ABD-PE halted the progression of autoimmune diseases in the system described herein and also concomitantly preserved normal adaptive immunity.

Methods.

Animal and Cell Lines.

Female C57BL/6 mice were purchased from The Jackson Laboratories. Female NOD mice were purchased from The Jackson Laboratories and bred in-house. The EL4 and B16-F10 (abbreviated as B16 cells) cell lines were purchased from the ATCC and maintained in DMEM medium with 10% FBS.

Generation of Expression Vectors for Recombinant Proteins.

Genes encoding αPD-1 (scFv), PE, or ABD-PE were synthesized by Biomatik. Genes were inserted into the pET25b (+) vector at the BseRI restriction sites. The configuration of the αPD-1 (scFv) was NH₂-V_{H}-Linker-V_{L}-COOH where V_{H} and V_{L} were the variable regions of the αPD-1 heavy chain and the αPD-1 light chain, respectively. Two mutations were introduced in the αPD-1, R45C; V_{L}:G104C. The linker inside the αPD-1 was (GGGGS)₃ (SEQ ID NO: 1). The linker was also inserted in between the ABD domain and the PE domain of ABD-PE. The coding genes of αPD-1-ABD-PE and αPD-1-PE were generated by fusing the synthesized genes together using a previously described protocol (Dong, S., et al., Engineering of a self-adjuvanted iTEP-delivered CTL vaccine. Acta Pharmacol Sin, 2017. 38(6): p. 914-923; and Dong, S., et al., A Comparison Study of iTEP Nanoparticle-Based CTL Vaccine Carriers Revealed a Surprise Relationship between the Stability and Efficiency of the Carriers. Theranostics, 2016. 6(5): p. 666-78). Meanwhile, a gene that encodes the linker was inserted between the genes of αPD-1 and ABD-PE, and between the genes of αPD-1 and PE. The linker gene was generated by annealing the sense and antisense oligonucleotides of the gene (Eurofins Genomics). A hexa-histidine tag (HisTag) was added at the NH₂-terminus of αPD-1-ABD-PE and its control proteins to facilitate the purification of these proteins; a linker, GGGGS (SEQ ID NO: 2), was inserted between the HisTag and the proteins. Accordingly, the coding gene of the HisTag was also fused to the coding genes of these proteins. The lengths of the finally resultant genes were confirmed by agarose gel electrophoresis after they were cleaved from pET25b (+) vector by double digestion with BamHI and XbaI. The sequences of these genes were verified by DNA sequencing (Genewiz).

Protein Expression and Purification.

The pET25b (+) vectors that harbor coding genes of αPD-1-ABD-PE, αPD-1, ABD-PE, and αPD-1-PE were transferred into competent Shuffle T7 E. coli cells (New England Biolabs). These transformed E. coli cells were cultured in LB broth at 32° C. until the optical density (OD₅₉₅) of the culture reached 0.6. At that point, the cultured cells were induced with 1 mM final concentration IPTG (Gold Biotechnology) for protein expressions that lasted 18 hours.

The cultured E. coli cells were harvested by centrifuging the culture at 4,700 rpm for 30 minutes. The collected cells were lysed by sonication using the 4-minute ON time. The cell samples were kept on ice during sonication, and 1 mM PMSF (Gold Biotechnology) was added to the cell lysate to inhibit protein degradation. After sonication, the supernatant of the cell lysate was collected by centrifuging the lysed samples at 20,000 g for 1 hour. Next, imidazole powder was added into the supernatant to reach a final imidazole concentration of 20 mM. At the same time, HisPur Ni-NTA resin (Thermo Fisher Scientific) was equilibrated with 10 mM imidazole in PBS. The equilibrated resin was then incubated with the supernatant for 1 hour at 4° C. on a rotator mixer. After the incubation, the mixture was loaded on a column, and the impurities and endotoxins were removed by washing the column with 60 mM imidazole that contained 1% Triton X-114. The wash was repeated until protein concentrations in the eluent were low (OD₂₈₀<0.01). The method to use 1% Triton X-114 for endotoxin removal was described previously (Zimmerman, T., et al., Simultaneous metal chelate affinity purification and endotoxin clearance of recombinant antibody fragments. J Immunol Methods, 2006. 314(1-2): p. 67-73). After this purification step, Triton X-114 was removed by washing the column with 50 column volumes of 60 mM imidazole. Finally, the desired proteins were eluted from the column with 300 mM imidazole in PBS (pH=8). Imidazole was removed by PD-10 desalting columns (GE Healthcare Life Sciences). The purity and residual endotoxin level of the product proteins were analyzed by SDS-PAGE and PYROGENT single test vials (Lonza, Allendale, N.J.).

SDS-PAGE Analysis of Proteins.

Five μg of αPD-1, ABD-PE, αPD-1-PE, and αPD-1-ABD-PE were first reduced by 2-Mercaptoethanol and denatured by heating the samples at 95° C. for 5 minutes. Then, these denatured samples were analyzed on a 4-15% gradient SDS-PAGE gel. After the electrophoresis, the gel was stained with Coomassie brilliant blue and photographed using an Alpha Innotech Fluorchem FC2 gel imaging system. The picture was processed by adjusting the brightness and contrast for the entire images. An open-source software GNU Image Manipulation Program (GIMP) was used to process the image.

Determination of the Binding Affinity of αPD-1-ABD-PE to EL4 Cells.

One million EL4 cells were incubated with Alexa Fluor 647-labeled αPD-1-ABD-PE and αPD-1 IgG at a series of different concentrations at 4° C. for 30 min. Then, the EL4 cells were analyzed by flow cytometry for PD-1⁺ fractions of EL4 cells. PD-1⁺ fraction of EL4 cells were plotted against protein concentrations and K_{d} values were obtained by fitting the curve using sigmoidal dose-response model in GraphPad V5. EC₅₀ values were used as the K_{d} values (Hunter, S. A. and J. R. Cochran, Cell-Binding Assays for Determining the Affinity of Protein-Protein Interactions: Technologies and Considerations. Methods Enzymol, 2016. 580: p. 21-44). 95% confidential intervals (95% CI) were calculated by GraphPad.

Generation of PD-1⁺ Primary T Cells.

Mouse splenocytes were harvested and cultured in 96-well plates at 2×10⁶/mL in complete RPMI-1640. Then, concanavalin A (Con A) (InvivoGen) was added into the cutlture at 6.25 μg/mL to stimulate PD-1 expression on primary T cells (Agata, Y., et al., Expression of the PD-1 antigen on the surface of stimulated mouse T and B lymphocytes. Int Immunol, 1996. 8(5): p. 765-72). After 72-hour stimulation, cells were collected. PD-1⁻ primary T cells were naïve splenocytes cultured in complete RPMI-1640 without Con A for 72 hours. These primary T cells were stained with aCD3-PE during later flow cytometry analysis.

Generation of PD-1⁺ Primary B Cells.

Mouse splenocytes were harvested and cultured in 96-well plates at 2×10⁶/mL in complete RPMI-1640. Then, 10 μg/mL AffiniPure F(ab′)₂ fragment of goat anti-mouse IgM, μ Chain Specific (Jackson ImmunoResearch, code 115-006-020) was added into culture to stimulate PD-1 expression on primary B cells (Agata, Y., et al., Expression of the PD-1 antigen on the surface of stimulated mouse T and B lymphocytes. Int Immunol, 1996. 8(5): p. 765-72). After 72-hour stimulation, the cells were collected. PD-1⁻ primary B cells were naïve splenocytes cultured in complete RPMI-1640 without anti-IgM for 72 hours. These primary B cells were stained with αB220-PE during later flow cytometry analysis.

Evaluation of Cell Binding and Endocytosis.

αPD-1-ABD-PE or ABD-PE were first labeled with Alexa Fluor 647 NHS Ester (Thermo Fisher Scientific). The labeled αPD-1-ABD-PE or ABD-PE (100 nM) were incubated with 0.5 million of cells separately. The incubation mixtures were maintained at 4° C. or 37° C. in FACS buffer (PBS with 0.1% FBS) for 30 minutes. After the incubation, unbound proteins were removed by centrifugation (350 g for 5 minutes). Last, the median fluorescence intensity (MFI) of these incubation mixtures were determined by flow cytometry with a BD FACSCanto Analyzer (BD Biosciences).

Specifically, for primary B cells, the MFI values of B-cell populations were analyzed. B-cell populations were identified by staining the cell mixture by αB220-PE.

For primary T cells, the MFI values of T-cell populations were analyzed. T-cell populations were identified by staining the cell mixture by aCD3-PE.

Binding Inhibition Study with PD-L1-Fc.

A PD-L1-Fc was used to compete surface PD-1 binding of αPD-1-ABD-PE. The PD-L1-Fc is a fusion protein between mouse PD-L1 and human Fc (Sino Biological, 50010-M03H). For concurrent incubation, the PD-L1-Fc was added to a final concentration of 500 nM with Alexa Fluor 647-labeled αPD-1-ABD-PE (100 nM final concentration) into 0.5 million cells and incubated for 30 min at 4° C. or 37° C. Then, unbound proteins were washed away by centrifugation (350 g for 5 minutes) in FACS buffer. The cells were analyzed by flow cytometry with a BD FACSCanto Analyzer (BD Biosciences). Human IgG at 500 nM was used as the control for PD-L1-Fc fusion.

In the experiment, surface-bound αPD-1-ABD-PE needs to be stripped. Alexa Fluor 647-labeled αPD-1-ABD-PE was first incubated with 0.5 million of cells for 30 min at 37° C., then PD-L1-Fc was added to a final concentration of 500 nM. The incubation mixtures were kept at 4° C. for additional 30 min before unbound proteins were washed away by centrifugation (350 g for 5 minutes) in FACS buffer. The cells were analyzed by flow cytometry with a BD FACSCanto Analyzer (BD Biosciences).

Generation of PD-1⁻ EL4 cell line. A single guide RNA (sgRNA) sequence in the exon 2 of the mouse PD-1 gene Pdcd1 (NC_000067.6) was chosen based on an algorithm described previously (Shalem, O., et al., Genome-scale CRISPR-Cas9 knockout screening in human cells. Science, 2014. 343(6166): p. 84-87). The sequence of sgRNA was CCTTGACACACGGCGCAATGACAGTGGCAT (SEQ ID NO: 17). The protospacer adjacent motif (PAM) was TGG. To target this sgRNA, two oligos were commercially synthesized (Integrated DNA Technologies): 5′-CACCGGACACACGGCGCAATGACAG-3′ (SEQ ID NO: 18) and 5′-AAACCTGTCATTGCGCCGTGTGTCC-3′ (SEQ ID NO: 19). The two oligos were annealed and ligated into the lentiviral vector lentiCRISPRv2-mCherry. High-title lentiviral supernatants were generated as previously described (Pear, W. S., et al., Production of high-titer helper free retroviruses by transient transfection. Proc Natl Acad Sci USA, 1993. 90(18): p. 8392-6) by co-transfection of HEK293T cells (ATCC) with the packaging plasmids pMD2.G (Addgene 12259) and psPAX2 (Addgene 12260). After EL4 cells were transduced with lentiviral supernatants, the transductants were stained with PE-Cy7 conjugated anti-PD-1 antibody (BioLegend, Clone: RMPI-30) and sorted for mCherry⁺ PD-1⁻ cells with FACSAria™ cell sorter (BD Biosciences) at the Flow Cytometry Core of the University of Utah. The EL4 and 293T cell lines were maintained in DMEM with 10% FBS.

Cytotoxicity Study.

For the cytotoxicity study with PD-1⁺ primary T cells, naïve splenocytes (concentration 2×10⁶/mL) were first stimulated with 6.25 μg/mL Con A for 24 hours. Then, αPD-1-ABD-PE or the control mixture were added into the wells without removing Con A. Two days after adding drugs, cells were harvested. Live cells were counted after stained with trypan blue. The percentage of PD-1⁺ primary T cells among these live cells were determined by flow cytometry after the live cells were stained with aCD3-PE (Biolegend), 3 μM DAPI (4′,6-Diamidino-2-Phenylindole, Dilactate) (Biolegend), and αPD-1-Alexa Fluor 647. Last, the number of live PD-1⁺ primary T cells were calculated by multiplying the number of total live cells and the percentage of PD-1⁺ primary T cells. For the cytotoxicity study with PD-1⁻ primary T cells, naïve splenocytes (concentration 2×10⁶/mL) were first culture in complete RPMI-1640 for 24 hours. Then, αPD-1-ABD-PE or the control mixture were added in the culture. Two days later, cells were harvested and analyzed same as described above. The total number of live cells, the percentage of PD-1⁻ primary T cells, as well as the number of live PD-1⁻ primary T cells were determined in the same way as that of PD-1⁺ primary T cells.

The cell viabilities were calculated for different concentrations of αPD-1-ABD-PE and the control mixture using the following equation. The viability data was fitted to a Sigmoidal dose-response model to determine IC₅₀ and 95% CI using GraphPad V5 (N=6).

The equation for cell viability:

Cell viability (%)=[PD-1⁺(or PD-1⁻) T cell number of treated well]/[PD-1⁺(or PD-1⁻) T cell number of untreated well].

The toxicity study with PD-1⁺ and PD-1⁻ primary B cells was the same as the one with T cells except that anti-mouse IgM was used to stimulate B cells and αB220-PE was used to stain B cells.

For the cytotoxic studies on EL4 cells and PD-1⁻ EL4 cells, EL4 cells or PD-1⁻ EL4 cells (10,000 cells/well) were seeded into 96-well plates. αPD-1-ABD-PE or control mixture were incubated with the cells in 100 μL medium at 37° C. for 72 hours. After the incubation, proliferations of these cell samples were determined by the CellTiter MTS assay (Promega), and the OD₄₉₀ of each treated cell sample was measured. The same measurement was also conducted for untreated cells (the live control) and the cells that were treated with 1% Triton X-100 (the dead control). These control cell samples were also cultured for 72 hours and their proliferations were determined the same as αPD-1-ABD-PE-treated cells. The cell viabilities were calculated for different concentrations of αPD-1-ABD-PE and the control mixture using the following equation. The viability data was fitted to a Sigmoidal dose-response model to determine IC50 and 95% CI using GraphPad V5 (N=6).

The equation for cell viability:

Cell viability (%)=(OD_{treated}−OD_{dead control})/(OD_{live control}−OD_{dead control}).

OD_{treated}: OD₄₉₀ of the αPD-1-ABD-PE or the control treated cells,

OD_{live control}: OD₄₉₀ of the live control,

OD_{dead control}: OD₄₉₀ of the dead control (Triton X-100 treated cells)

In Vivo EL4 Depletion.

Five million of EL4 (PD-1-positive) cells were injected into C57BL/6 mice intravenously through their tail veins. The injected mice were randomly assigned into 3 groups. Two hours later, the three groups were treated with a single dose of αPD-1-ABD-PE (5 mg/kg), a mixture of αPD-1 (scFv, 2.5 mg/kg) and ABD-PE (2.5 mg/kg), or PBS intraperitoneally. At 72 hours after the treatments, these mice were euthanized, and circulating lymphocytes were collected from the mice. The cells were stained with Alexa Fluor 647-labeled αPD-1 and analyzed by flow cytometry with a BD FACSCanto Analyzer. The fractions of PD-1-positive cells among circulating lymphocytes were determined through the analysis.

Albumin-Binding Study.

MSA (>96% pure, Sigma) and HSA (>97% pure, Sigma) were dissolved in PBS. 17.4 μg of αPD-1-ABD-PE or 15.9 μg of αPD-1-PE were incubated with 20.0 μg MSA (1:1 molar ratio) in 20 μL PBS at room temperature for 15 minutes. After incubation, the samples were analyzed by native PAGE. The PAGE gel was stained by Coomassie brilliant blue and photographed using an Alpha Innotech Fluorchem FC2 gel imaging system. The picture was processed by adjusting the brightness and contrast for the entire images. An open-source software GNU Image Manipulation Program (GIMP) was used to process the image.

Regarding HSA, the amount of proteins used is the same as MSA incubation experiment. After incubation, the samples were analyzed as described above.

Pharmacokinetics (PK). Five nmole of αPD-1-ABD-PE or αPD-1-PE that were labeled by NHS-Fluorescein (Thermo Fisher Scientific Inc) were injected intraperitoneally into C57BL/6 mice. At each pre-determined time point (0.25, 1, 2, 4, 8, 12, 24, and 48 hours after dosing), a cohort of three mice was sacrificed, and the blood were collected for quantification of protein concentration. Plasma concentrations of the proteins were determined according to the fluorescence intensity (Ex 494 nm/Em 518 nm). The pharmacokinetics of both proteins was analyzed using non-compartmental analysis (Phoenix WinNolin software, version 8.0, Pharsight Corporation, CA, USA) (Zhang, Y., et al., PKSolver: An add-in program for pharmacokinetic and pharmacodynamic data analysis in Microsoft Excel. Comput Methods Programs Biomed, 2010. 99(3): p. 306-14). PK parameters CL, t_{1/2}, AUC₀₋ₜ, and V_{d} were calculated.

Disease Delay Study with a Spontaneous T1D Model.

Twelve-week-old female NOD mice were randomly assigned into three groups. The three groups were treated intraperitoneally with αPD-1-ABD-PE (5 mg/kg), a mixture of αPD-1 (scFv, 2.5 mg/kg) and ABD-PE (2.5 mg/kg), or PBS weekly until the mice were found to be diabetic. Blood glucose levels of these mice were monitored twice weekly using a OneTouch UltraMini Blood Glucose Meter (LifeScan, Inc). The T1D onset was confirmed for a mouse when blood glucose concentrations of the mouse were greater than 250 mg/dL in two consecutive measurements. Diabetes-free survival was analyzed by the Kaplan-Meier method followed the Log-rank (Mantel-Cox) test using GraphPad Prism V5.

Disease Delay Study with a Cyclophosphamide (CP)-Accelerated T1D Model.

Ten-week-old female NOD mice were injected intraperitoneally with CP (Santa Cruz Biotechnology) at 200 mg/kg. Two days later, these mice were randomly assigned into three groups. The three groups were treated intraperitoneally with αPD-1-ABD-PE (5 mg/kg), a mixture of αPD-1 (scFv, 2.5 mg/kg) and ABD-PE (2.5 mg/kg), or PBS every other day for a total of five treatments. The diabetes-free survival data were generated and analyzed as described above.

Disease Delay Study with a αPD-1-Accelerated T1D Model.

Ten-week-old female NOD mice were randomly assigned into three groups. The three groups were treated intraperitoneally with αPD-1-ABD-PE (5 mg/kg), a mixture of αPD-1 (scFv, 2.5 mg/kg) and ABD-PE (2.5 mg/kg), or PBS every other day for totally five treatments. Two days after the last dosing, 0.5 mg/mouse αPD-1 IgG (clone RMP1-14) (Zhao, P., et al., An Anti-Programmed Death-1 Antibody (alphaPD-1) Fusion Protein That Self-Assembles into a Multivalent and Functional alphaPD-1 Nanoparticle. Mol Pharm, 2017. 14(5): p. 1494-1500) was administered intraperitoneally to the mice. Two days later, these mice received four additional doses of αPD-1 IgG with a two-day interval at a dose level of 0.25 mg/mouse/injection. The diabetes-free survival data were collected and analyzed as described above.

Examination of Immune Cells in Pancreases, Blood, and Peripheral Lymphatic Organs of Pre-Diabetic NOD Mice.

Eighteen-week-old, prediabetic, female NOD mice were randomly assigned into three groups. The three groups were treated intraperitoneally with one dose of αPD-1-ABD-PE (5 mg/kg), a mixture of αPD-1 scFv (2.5 mg/kg) and ABD-PE (2.5 mg/kg) or PBS. Three days later, these mice were euthanized and perfused with 30 mL PBS. Pancreases were collected from these mice and a single cell preparation was generated from each of the pancreases by mincing the pancreas tissues with scissors and digesting the tissues with 1 mg/mL collagenase IV at 37° C. for 30 minutes as described previously (Gregori, S., et al., Dynamics of pathogenic and suppressor T cells in autoimmune diabetes development. J Immunol, 2003. 171(8): p. 4040-7). The pancreatic cell preparations were filtered through a nylon mesh and then dead cells were first stained by 3 μM DAPI for 10 min, and then washed 3 times by a centrifugation (300 g for 5 minutes) to remove DAPI. Cells were then stained with αB220-PE, aCD8-PE, aCD3-PECy7, aCD4-FITC, αFOXP3-PE (antibodies were purchased from Biolegend), and αPD-1-Alexa Fluor 647 was made in house. Lastly, 400,000 live cells per pancreatic cell preparation were analyzed by flow cytometry with a BD FACSCanto Analyzer.

To analyze immune cells in blood and peripheral lymphatic organs, blood, spleens, lymph nodes (accessory axillary lymph nodes and subiliac lymph nodes) were collected from the aforementioned mice. Red blood cells in these samples were lysed with the Ammonium-Chloride-Potassium (ACK) Lysing Buffer. The remaining cells were pelleted by a centrifugation at 300 g for 5 minutes, and stained by DAPI (3 μM), αB220-PE, αCD8-PE, αCD4-PECy7, αCD3-FITC, αFOXP3-PE, and αPD-1-Alexa Fluor 647. Lastly, 10,000 live cells per cell preparation were analyzed by flow cytometry with a BD FACSCanto Analyzer.

Disease Reversal Study in an EAE Model.

Ten-week-old female C57BL/6 mice were subcutaneously immunized with 0.2 mg MOG₃₅₋₅₅ peptide (MEVGWYRSPFSRVVHLYRNGK (SEQ ID NO: 9), Biomatik) per mouse. The peptide was emulsified in 0.2 mL CFA (Sigma). Four hours later, mice received intraperitoneally 200 ng pertussis toxin (List Biological Labs, Inc). And 24 hours later, mice received an additional 200 ng pertussis toxin. The immunized mice were examined for their paralysis symptoms every other day from the 9th day after the immunization. The symptoms were scored based on a common standard (Chitnis, T., et al., Effect of targeted disruption of STAT4 and STAT6 on the induction of experimental autoimmune encephalomyelitis. Journal of Clinical Investigation, 2001. 108(5): p. 739-747): 1.0, limp tail or isolated weakness of gait without limp tail; 2.0, partial hind leg paralysis; 3.0, total hind leg or partial hind and front leg paralysis; 4.0, total hind leg and partial front leg paralysis; 5.0, moribund or dead animal. At the same time, the immunized mice were randomly assigned into three groups; each group would receive one of the three treatments: αPD-1-ABD-PE (5 mg/kg), a mixture of αPD-1 (scFv, 2.5 mg/kg) and ABD-PE (2.5 mg/kg), or PBS. When an immunized mouse developed severe EAE (clinical score 3.0), this mouse would be immediately treated according to its group assignment. The treated mice were continuously monitored until they reached their humane endpoints or the end of the study.

Examination of Immune Cells in Pancreases, Blood, and Peripheral Lymphatic Organs of Mice with EAE.

Ten-week-old female C57BL/6 mice were induced for EAE as described above. After the induced mice developed severe EAE (clinical score 3.0), the mice were randomly assigned into three groups that received αPD-1-ABD-PE (5 mg/kg), a mixture of αPD-1 (scFv, 2.5 mg/kg) and ABD-PE (2.5 mg/kg), or PBS intraperitoneally. Three days later, these mice were euthanized and perfused with 30 mL PBS after blood was collected. The spinal cord and brain were collected from these mice, and were subsequently minced digested as described above. A single cell preparation was generated by filtering the processed tissues of one mouse through a nylon mesh. Mononuclear cells were enriched in the preparations by passing the preparations through a discontinuous 30%:70% Percoll gradient as described previously (McMahon, E. J., et al., Epitope spreading initiates in the CNS in two mouse models of multiple sclerosis. Nat Med, 2005. 11(3): p. 335-9; and Pino, P. A. and A. E. Cardona, Isolation of brain and spinal cord mononuclear cells using percoll gradients. J Vis Exp, 2011(48)). The resultant cell preparations were stained and analyzed by flow cytometry. Dead cells were first stained by 3 μM DAPI for 10 min, and then washed 3 times by a centrifugation (300 g for 5 minutes) to remove DAPI. Then cells were stained with αB220-PE, αCD8-PE, αCD3-PECy7, αCD4-FITC, αFOXP3-PE (antibodies were purchased from Biolegend), I-Ab MOG₃₈₋₄₉-PE tetramer (provided by NIH tetramer care facility), and αPD-1-Alexa Fluor 647 which was made in house. Lastly, 200,000 live cells per cell preparation were analyzed by flow cytometry with a BD FACSCanto Analyzer.

To analyze immune cells in blood and peripheral lymphatic organs, blood, spleens, lymph nodes (accessory axillary lymph nodes and subiliac lymph nodes) were collected from the aforementioned mice. Red blood cells in these samples were lysed with the Ammonium-Chloride-Potassium (ACK) Lysing Buffer. The remaining cells in the samples were pelleted by a centrifugation at 300 g for 5 minutes, and then cells were stained by DAPI (3 μM), αB220-PE, αCD8-PE, αCD4-PECy7, αCD3-FITC, αFOXP3-PE, and αPD-1-Alexa Fluor 647. Lastly, 10,000 live cells per cell preparation were analyzed by flow cytometry with a BD FACSCanto Analyzer.

Fraction Analysis of Peripheral Lymphocytes in Healthy NOD and C57BL/6 Mice.

Ten-week-old female NOD mice were randomly assigned into 4 groups and treated with one dose of αPD-1-ABD-PE (5 mg/kg), a mixture of αPD-1 (scFv, 2.5 mg/kg) and ABD-PE (2.5 mg/kg), PBS, or CP (200 mg/kg) (Santa Cruz Biotechnology) intraperitoneally. These mice were euthanized 24 hours later. Blood samples and splenocytes were collected from the mice, and red blood cells in these samples were lysed with the Ammonium-Chloride-Potassium (ACK) Lysing Buffer. The remaining cells in the samples were pelleted by a centrifugation at 300 g for 5 minutes, and stained by αB220-APC (Biolegend), αCD4-PE (Biolegend), or αCD8α-PE (Santa Cruz Biotechnology, Inc). The B220⁺, CD4⁺, and CD8⁺ fractions in the pellets were analyzed by flow cytometry on a BD FACSCanto Analyzer. The same experiment was conducted on 10-week-old female C57BL6 mice.

Evaluation of Humoral Responses.

Ten-week-old female NOD mice were randomly assigned into 4 groups and treated once or five times with αPD-1-ABD-PE (5 mg/kg), a mixture of αPD-1 (scFv, 2.5 mg/kg) and ABD-PE (2.5 mg/kg), PBS, or CP (200 mg/kg) (Santa Cruz Biotechnology) intraperitoneally. Two days after the last treatment, mice were immunized with 100 μg DNP-Ficoll (Biosearchtech) per mouse intraperitoneally. Five days after the immunization, serum samples were collected from these mice to determine anti-DNP IgM levels using an ELISA as previously described with some modification (Turner, M. J., et al., Immune status following alemtuzumab treatment in human CD52 transgenic mice. J Neuroimmunol, 2013. 261(1-2): p. 29-36). Specifically, serum dilutions from 100 to 10⁶ folds were applied to ELISA plates pre-coated with DNP-BSA (Thermo Fisher Scientific Inc) (1 μg/well, 4° C., overnight). After unbound IgM was washed away, a secondary antibody, anti-mouse IgM (μ chain)-HRP (Jackson ImmunoResearch) (1:5000 diluted in PBS with 1% BSA) was added into the plates. After a 1-hour incubation at room temperature, the unbound secondary antibody was washed away, and a TMB substrate (Biolegend) was added to the plates. The plates were then incubated in the dark at room temperature for 20 minutes for the colorimetric reaction to develop. The reaction was stopped with 2 M H₂SO₄. Both OD₄₅₀ and OD₅₇₀ (background) were measured. The same diluted serum samples were also applied to ELISA plates pre-coated with the BSA control (Sigma) (1 μg/well, 4° C., overnight) in order to determine the non-specific binding of IgM to the plates. All other procedures were the same.

The same experiment was also performed with 10-week-old female C57BL/6 mice.

Evaluation of CTL Responses.

Ten-week-old female NOD mice were randomly assigned into 4 groups and treated with a single dose of αPD-1-ABD-PE (5 mg/kg), a mixture of αPD-1 (scFv, 2.5 mg/kg) and ABD-PE (2.5 mg/kg), PBS, or CP (200 mg/kg) (Santa Cruz Biotechnology) intraperitoneally. Two days later, these mice were subcutaneously immunized with 10 nmole of TYQRTRALV (SEQ ID NO: 16) (Influenza A (PR8) NP 147-155, Biomatik) per muse. The peptide was emulsified in IFA (Sigma). Seven days after the first immunization, a second immunization was administered. Ten days after the second immunization, these mice were sacrificed, and an ELISPOT was performed with splenocytes collected from these mice as previously described (Dong, S., et al., Engineering of a self-adjuvanted iTEP-delivered CTL vaccine. Acta Pharmacol Sin, 2017. 38(6): p. 914-923; and Dong, S., et al., A Comparison Study of iTEP Nanoparticle-Based CTL Vaccine Carriers Revealed a Surprise Relationship between the Stability and Efficiency of the Carriers. Theranostics, 2016. 6(5): p. 666-78).

In a similar manner, ten-week-old female C57BL/6 mice were treated and immunized with 10 nmole of SIINFEKL (SEQ ID NO: 17) (OVA 257-264). SIINFEKL-specific CTL responses were evaluated as described above.

Results.

αPD-1-ABD-PE Selectively Binds and Penetrate PD-1⁺ Cells.

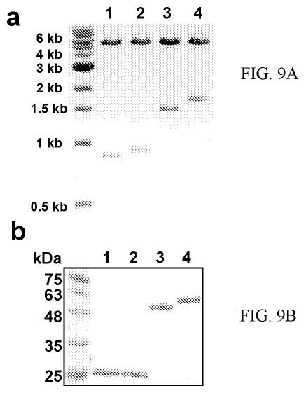

αPD-1-ABD-PE has three functional components: αPD-1 (scFv), ABD, and PE (FIG. 8a). The sequences of the ABD and PE have been previously published (Onda, M. et al. Recombinant immunotoxin against B-cell malignancies with no immunogenicity in mice by removal of B-cell epitopes. Proc Natl Acad Sci USA 108, 5742-5747 (2011); and Jonsson, A., Dogan, J., Herne, N., Abrahmsen, L. & Nygren, P. A. Engineering of a femtomolar affinity binding protein to human serum albumin. Protein Eng Des Sel 21, 515-527 (2008)). An αPD-1 clone, RMP1-14, which is a rat anti-mouse PD-1 mAb (IgG2a, κ) (Yamazaki, T. et al. Blockade of B7-H1 on macrophages suppresses CD4+ T cell proliferation by augmenting IFN-gamma-induced nitric oxide production. J Immunol 175, 1586-1592 (2005)) was cloned. Based on the sequencing results, the αPD-1 was designed to contain two mutations, V_{H} R45C and V_{L} G104C (FIG. 8b). These two cysteines were introduced to form a disulfide bond and enhance stability of the αPD-1 (Brinkmann, U. in Antibody Engineering. (eds. R. Kontermann & S. Dübel) 181-189 (Springer Berlin Heidelberg, Berlin, Heidelberg; 2010)). A linker, (GGGGS)₃, was used to connect the αPD-1, the ABD, and the PE. Three control proteins were also designed: αPD-1, ABD-PE, and αPD-1-PE (FIG. 8a and Table 2). These proteins have a histidine-tag (HisTag) to facilitate purification. The coding genes for αPD-1-ABD-PE and the controls were cloned into pET25b (+) vector. The sizes of these genes were confirmed by agarose gel electrophoresis (FIG. 9a). These genes were sequenced and confirmed to have the designed sequences. αPD-1-ABD-PE and the controls were produced and purified as soluble proteins from E. coli (Shuffle T7) that harbored the expression vectors. The yield was approximately 0.3 mg/L culture for each of these proteins. The purity and size of these proteins were examined by SDS-PAGE (FIG. 9b). The sizes of these proteins reflected by SDS-PAGE results were consistent with their predicted theoretical molecular weights (Table 2); e.g. αPD-1-ABD-PE migrated slightly lower than the 63 kDa marker, which agreed with its theoretical molecular weight, 57.9 kDa. Endotoxin were removed from the purified proteins to levels below 0.1 unit/mg protein.

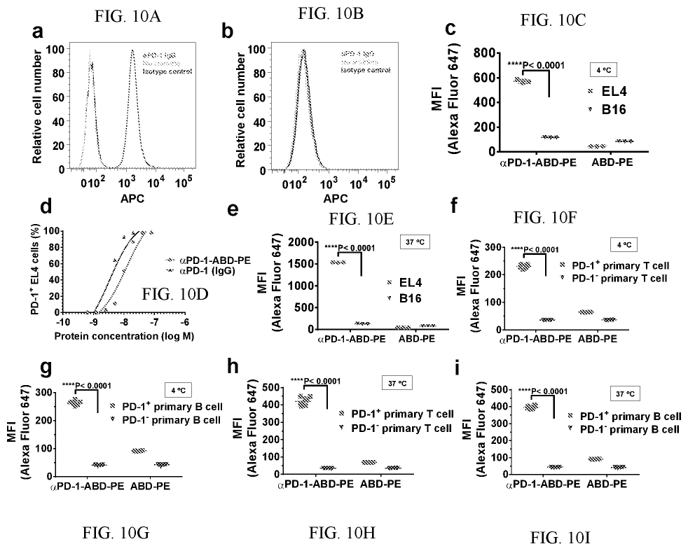

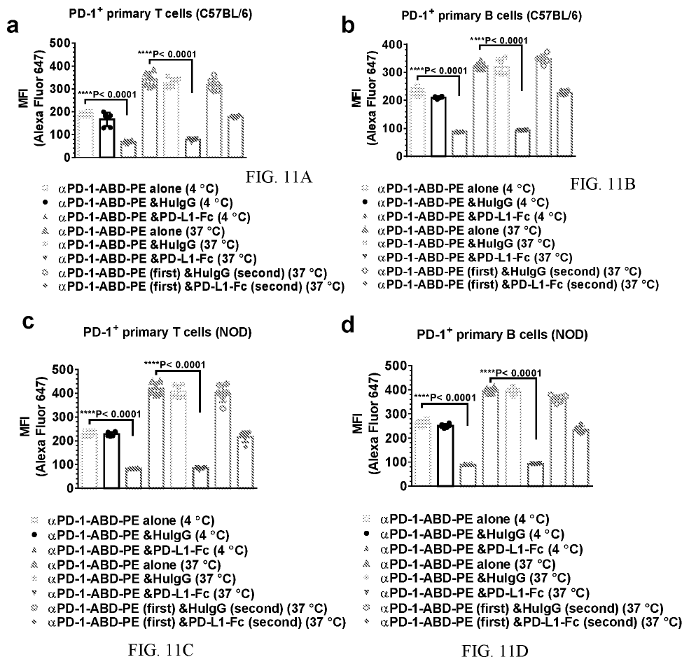

It was found that αPD-1-ABD-PE selectively binds to PD-1⁺ cells with three pairs of PD-1⁺ and PD-1⁻ cells: PD-1⁺ and PD-1⁻ primary T cells, PD-1⁺ and PD-1⁻ primary B cells, EL4 (PD-1⁺) (Hirano, F. et al. Blockade of B7-H1 and PD-1 by monoclonal antibodies potentiates cancer therapeutic immunity. Cancer research 65, 1089-1096 (2005)) and B16 (PD-1⁻) cells (FIG. 10a-b). Alexa Fluor 647-labeled αPD-1-ABD-PE was incubated with PD-1⁺ and PD-1⁻ cells, separately at 4° C. (30 min) whereby cell surface binding was allowed but internalization by cells was inhibited. After the incubation, the mean MFI of the PD-1⁺ primary T cells was more than 4 times of that of PD-1⁻ primary T cells (188.33±5.92 versus 43.45±2.98; FIG. 8c); the mean MFI of the PD-1⁺ primary B cells was more than 5 times of that of PD-1⁻ primary B cells (229.67±12.45 versus 43.72±2.46; FIG. 8d); the mean MFI of EL4 cells was more than 3 times of that of B16 cells (571.70±12.12 versus 116.00±1.61; FIG. 10c). In contrast, both the PD-1⁺ and the PD-1⁻ cells had low MFIs after similar incubations with labeled ABD-PE (FIG. 8c-d, FIG. 10c). Through a dose-responsive binding assay, it was found that αPD-1-ABD-PE had an apparent binding affinity of 12.0 nM (95% CI=8.7˜16.5 nM), which was greater than that intact αPD-1 (3.4 nM, 95% CI=2.2˜5.2 nM; FIG. 10d).

Labeled αPD-1-ABD-PE was also incubated with the PD-1⁺ and the PD-1⁻ cells, separately, at 37° C. (30 min) whereby both cell surface binding and cellular uptake were allowed to progress. After the incubation, the mean MFI of the PD-1⁺ primary T cells, as measured by flow cytometry, was more than 8 times of that of PD-1⁻ primary T cells (341.17±31.97 versus 42.13±2.00; FIG. 8e); the mean MFI of the PD-1⁺ primary B cells was more than 7 times of that of PD-1⁻ primary B cells (321.17±11.63 versus 41.98±1.97; FIG. 80; the mean MFI of the EL4 cells after the incubation was approximately 12 times of that of the B16 cells (1532.70±4.99 versus 130.00±2.55; Figure S2e). In contrast, both the PD-1⁺ and the PD-1⁻ cells had low MFIs after incubation with labeled ABD-PE at 37° C. (FIG. 8e-f, FIG. 10e). These data, together with the data generated at 4° C., suggest that αPD-1-ABD-PE specifically binds to and penetrates PD-1⁺ cells and αPD-1 directs the specificity. PD-1 was found to shuttle its binding partners into cells and rapidly complete the transport, based on the fact that, 1) the mean MFI of EL4 cells after the 37° C. incubation with αPD-1-ABD-PE was approximately several fold higher than the mean after the 4° C. incubation, and 2) both incubations were 30 minutes. It is worth noting that the same conclusion was reached using primary lymphocytes from both C57BL/6 (FIG. 8 c-f) and NOD (FIG. 10 f-i) mice.

An additional method was used to establish the role of PD-1 in the binding and internalization of αPD-1-ABD-PE to PD-1⁺ cells. Here, a fusion protein of the ligand 1 of programmed death 1 protein and a human IgG Fc domain (PD-L1-Fc)²⁷ was utilized and a competitive binding assay was performed (FIG. 8g, FIG. 11). It was found that concurrent incubation of αPD-1-ABD-PE and PD-L1-Fc with PD-1⁺ cells abolished the binding and internalization of αPD-1-ABD-PE into PD-1⁺ cells. Specifically, in the presence of PD-L1-Fc, the mean MFI of EL4 cells after the concurrent incubation at 4° C. was approximately 8-time smaller than the MFI of the cells incubated with αPD-1-ABD-PE alone (74.58±0.44 versus 583.80±6.76, P<0.0001; FIG. 8g); and at 37° C., the mean MFI resulting from the concurrent incubation was decreased by approximately by 25 times as compared to that from the single incubation (74.52±0.63 versus 1895.00±34.82, P<0.0001; FIG. 8g). There data corroborate the results presented above that the binding and internalization of αPD-1-ABD-PE into PD-1⁺ cells were directed by PD-1. Interestingly, when EL4 cells were first incubated with αPD-1-ABD-PE at 37° C. for 30 min and then with PD-L1-Fc added in the incubation mixture, PD-L1-Fc partially decreased the mean MFI of the EL4 cells, from 1895.00±34.82 to 1320.00±120.30 (FIG. 8g). The partial decrease may be because the PD-L1-Fc displaced surface bound αPD-1-ABD-PE but some αPD-1-ABD-PE has entered EL4 cells prior to the addition of PD-L1-Fc and, consequently, cannot be displaced by PD-L1-Fc. This observation is proof that αPD-1-ABD-PE enters PD-1⁺ cells. The conclusion that PD-1-mediated internalization is likely common to PD-1⁺ cells of different types and origins since the results are consistent between PD-1⁺ primary T and B cells and between cells collected from both C57BL/6 and NOD mice (FIG. 11).

αPD-1-ABD-PE Specifically Depletes PD-1⁺ Cells Both In Vitro and In Vivo.

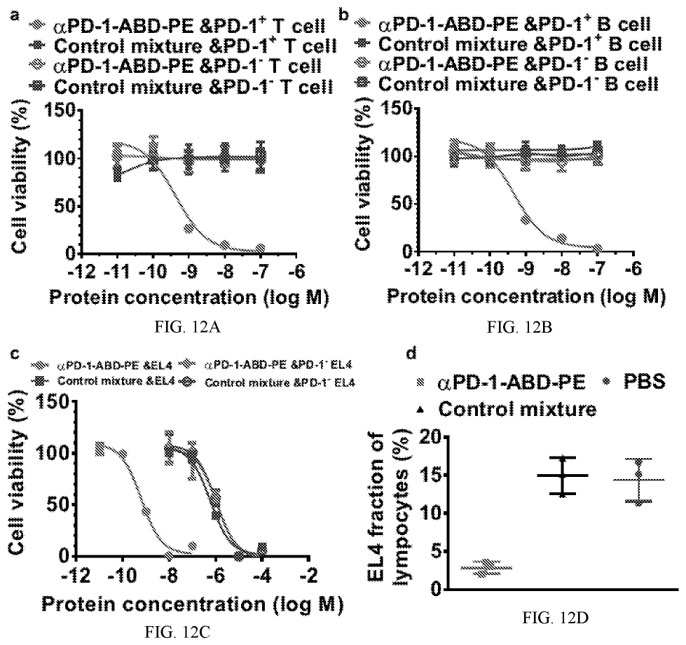

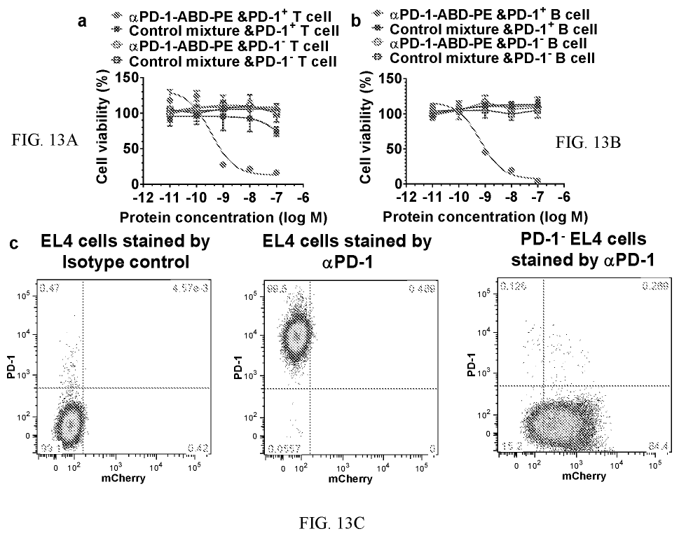

In vitro, αPD-1-ABD-PE was found to be at least hundred-time more lethal for PD-1⁺ primary lymphocytes than for PD-1⁻ primary lymphocytes: the IC50 of αPD-1-ABD-PE for PD-1+ primary T cells was 0.43 nM (95% CI=0.27˜0.69 nM) while αPD-1-ABD-PE did not show detectable cytotoxicity to PD-1⁻ primary T cells up to 100 nM (FIG. 12); the IC₅₀ of αPD-1-ABD-PE for PD-1⁺ primary B cells was 0.47 nM (95% CI=0.34˜0.63 nM) while αPD-1-ABD-PE did not show toxicity to PD-1⁻ primary B cells up to 100 nM (FIG. 12b). A control mixture of αPD-1 and ABD-PE did not show cytotoxicity to either PD-1⁺ or PD-1⁻ primary lymphocytes up to 100 nm (FIG. 12a-b). The drastically different toxicities between αPD-1-ABD-PE and the control mixture indicated the cytotoxicity of αPD-1-ABD-PE is due to a contiguous linkage between αPD-1 and ABD-PE. The same results were found in primary lymphocytes from both C57BL/6 (FIG. 12a-b) and NOD mice (FIG. 13a-b)

To pinpoint the role of αPD-1 in the selective cytotoxicity of αPD-1-ABD-PE to PD-1⁺ cells, a PD-1 knockout EL4 cell line (PD-1⁻ EL4) was generated using CRISPER/Cas9 (FIG. 13c). It was found that αPD-1-ABD-PE was more than 1000-time more toxic to wildtype EL4 cells than to PD-1⁻ EL4 cells (IC50=0.64 nM 95% CI=0.49˜0.83 nM versus IC₅₀=1120 nM 95% CI=702.7˜1773.0 nM; FIG. 12c). In contrast, a control mixture of αPD-1 and ABD-PE had little or no cytotoxicity to both cell lines (IC50=590 nM 95% CI=458.7760.6 nM for wildtype EL4 cells; IC50=1060 nM 95% CI=747.71514.0 nM for PD-1⁻ EL4 cells). Together, these results point to a clear role of PD-1 in the PD-1⁺ cell selective toxicity of αPD-1-ABD-PE.

αPD-1-ABD-PE was also found to effectively deplete PD-1⁺ cells in vivo. Specifically, a single dose of αPD-1-ABD-PE reduced the fraction of adoptively transferred EL4 cells to 2.86±0.46% among circulating lymphocytes in the hosts; while mice treated with the mixture of αPD-1 and ABD-PE had unchanged EL4 cell fraction as compared to the PBS-treated mice (14.93±1.36% versus 14.37±1.60%; FIG. 12d). The radically different cell depletion results between αPD-1-ABD-PE and the control mixture, again, confirm the importance of the linkage between αPD-1 and ABD-PE.

The ABD Increases the Plasma Exposure of αPD-1-ABD-PE.

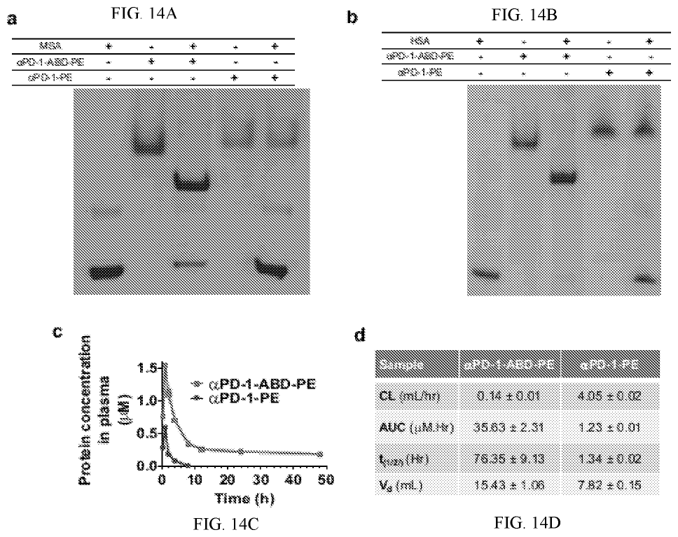

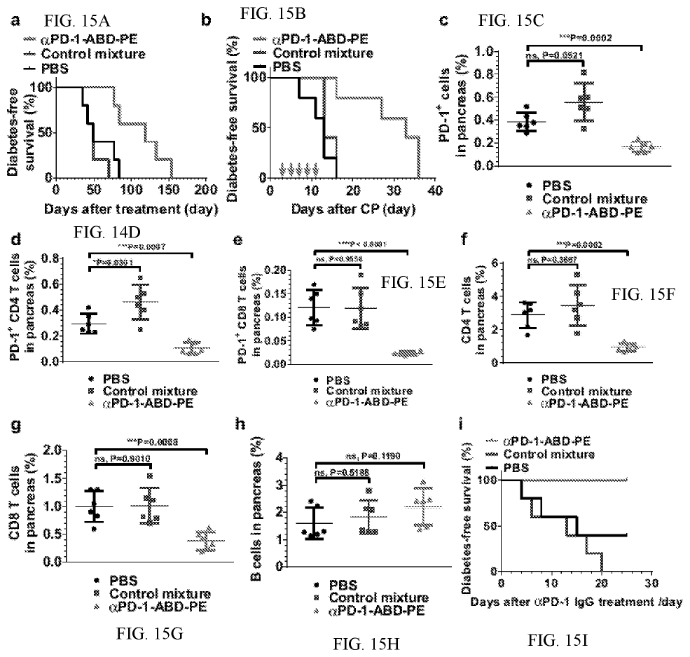

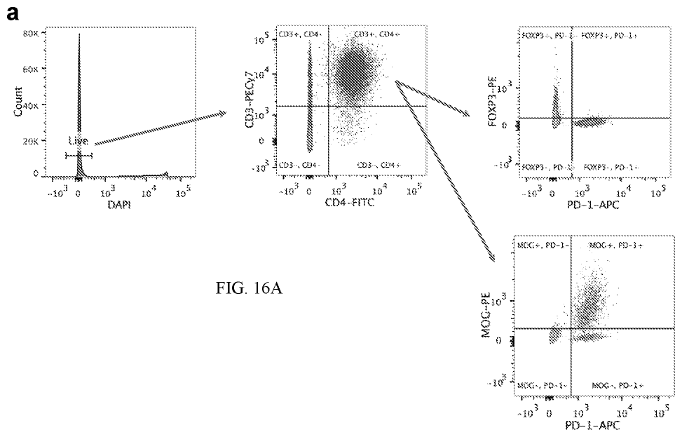

The function of the ABD in αPD-1-ABD-PE was also examined. According to native PAGE results, there was a clear association of αPD-1-ABD-PE with mouse serum albumin (MSA, FIG. 14a) and human serum albumin (HSA, FIG. 14b). In contrast, αPD-1-PE did not associate with either MSA or HSA. These data suggest that the ABD component of αPD-1-ABD-PE retains its albumin-binding capacity and that the interaction between αPD-1-ABD-PE and albumin are due to the presence of the ABD. Next, the pharmacokinetics of αPD-1-ABD-PE and αPD-1-PE was examined after intraperitoneally dosing mice at 5.0 nmol per mouse (FIG. 14c). Based on a non-compartmental analysis, αPD-1-ABD-PE has a systemic clearance (CL) that was approximately 30-fold less than that of αPD-1-PE (0.14±0.01 mL/hr versus 4.05±0.02 mL/hr), resulting in 30-time higher plasma exposure (area under curve, AUC₀₋ₜ); αPD-1-ABD-PE has a 57-fold longer terminal half-life (t_{(1/2)}) than αPD-1-PE (76.35±9.13 hr versus 1.34±0.02 hr). αPD-1-ABD-PE was also noted to be superior to αPD-1-PE in volume distribution (V_{d}) (FIG. 14d). These data demonstrate that the ABD in αPD-1-ABD-PE improved the PK of the construct, which was expected to benefit the efficacy of αPD-1-ABD-PE as poor PK was reported as an efficacy-limiting factor of previously developed immunotoxins (Bera, T. K., Onda, M., Kreitman, R. J. & Pastan, I. An improved recombinant Fab-immunotoxin targeting CD22 expressing malignancies. Leukemia research 38, 1224-1229 (2014).

αPD-1-ABD-PE Delays the Onset of T1D.

Female non-obese diabetic (NOD) mice are prone to develop T1D (Fife, B. T. et al. Insulin-induced remission in new-onset NOD mice is maintained by the PD-1-PD-L1 pathway. J Exp Med 203, 2737-2747 (2006)). Twelve week old female NOD mice that did not yet show hyperglycemia were treated with weekly injections of either, 1) αPD-1-ABD-PE, 2) PBS, or 3) a mixture of αPD-1 and ABD-PE. αPD-1-ABD-PE treatment markedly delayed the onset of T1D (hyperglycemia) as compared to PBS and the control mixture treatment. The median T1D-free survival time of the PBS-treated mice and the control mixture-treated mice were both 49 days after the treatment (P=0.3480 between the two treatments). In contrast, the median T1D-free survival time of the αPD-1-ABD-PE-treated mice was 119 days after the treatment (FIG. 15a; P=0.0180 versus PBS treatment).

The delaying effect of αPD-1-ABD-PE in a pathologically accelerated T1D model was also examined. Ten-week old female NOD mice were pre-treated with cyclophosphamide (CP) to expedite the onset of T1D due to CP's ability to ablate regulatory T-cells (Tregs) in NOD mice (Brode, S., Raine, T., Zaccone, P. & Cooke, A. Cyclophosphamide-induced type-1 diabetes in the NOD mouse is associated with a reduction of CD4+CD25+Foxp3+ regulatory T cells. J Immunol 177, 6603-6612 (2006)). These CP pre-treated mice were subsequently treated with either 1) αPD-1-ABD-PE, 2) PBS, or 3) a mixture of αPD-1 and ABD-PE. Both the PBS-treated and the mixture control-treated mice had a short median T1D-free survival of 13 days. In contrast, the αPD-1-ABD-PE-treated mice had a significantly longer median T1D free survival, 33 days (FIG. 15b; P=0.0068 versus PBS; P=0.0094 versus the mixture control). These data, taken together with the spontaneous T1D data, demonstrate that αPD-1-ABD-PE inhibits the development of T1D.

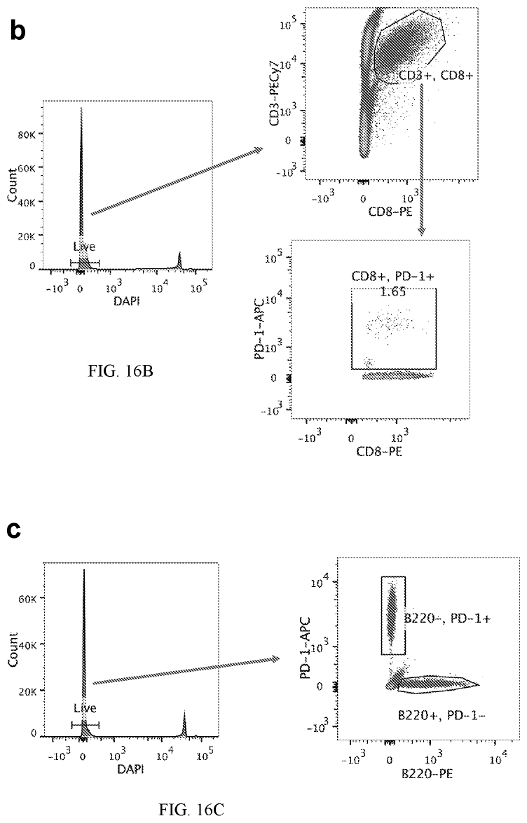

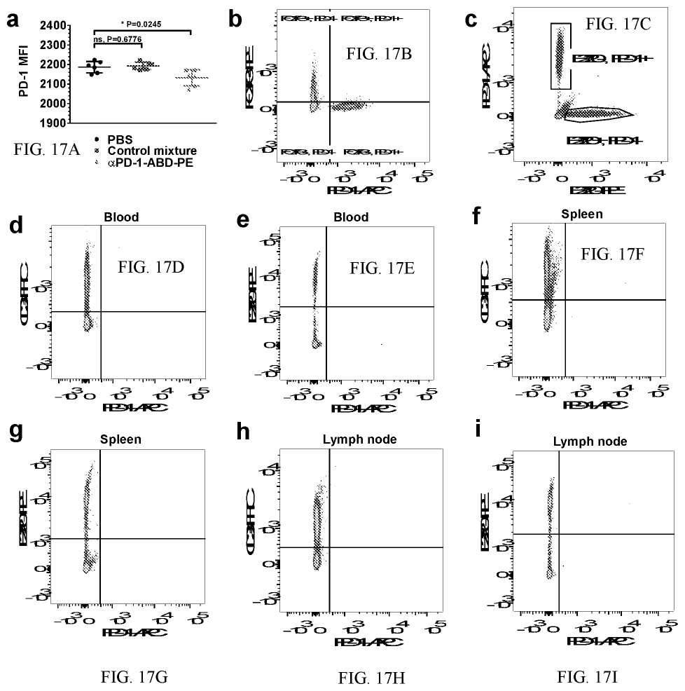

Since the pancreatic infiltrations of PD-1⁺ cells and lymphocytes especially CD4+ lymphocyte parallel the progression of T1D in NOD mice (Ansari, M. J. et al. The programmed death-1 (PD-1) pathway regulates autoimmune diabetes in nonobese diabetic (NOD) mice. J Exp Med 198, 63-69 (2003); Fife, B. T. et al. Insulin-induced remission in new-onset NOD mice is maintained by the PD-1-PD-L1 pathway. J Exp Med 203, 2737-2747 (2006); and Wang, J. et al. Establishment of NOD-Pdcd1−/− mice as an efficient animal model of type I diabetes. Proc Natl Acad Sci USA 102, 11823-11828 (2005)), it was investigated whether αPD-1-ABD-PE treatment would diminish the PD-1⁺ cell and lymphocyte infiltrations (FIG. 16). Indeed, even a single dose αPD-1-ABD-PE significantly reduced the fraction of PD-1⁺ cells in pancreases as compared to PBS (0.17±0.02% versus 0.39±0.03%, P=0.0002; FIG. 15c). In contrast, the control mixture did not reduce the fraction of PD-1⁺ cells (0.56±0.07%, P=0.052). When these PD-1⁺ cells were compared for the level of the PD-1 expression (MFI), the PD-1⁺ cells survived the αPD-1-ABD-PE treatment had slightly but statistically significantly lower MFI (2132.17±16.82, P=0.025) than the PD-1⁺ cells survived the PBS treatment (2187.00±12.12, FIG. 17a). There was no significant difference between the cells of the PBS and the control mixture treatments. The result may be due to a preferential target of αPD-1-ABD-PE to PD-1^{high} cells over PD-1^{low} cells. Further, αPD-1-ABD-PE also significantly reduced the fractions of PD-1⁺ CD4 T and PD-1⁺ CD8 T cells in the pancreas as compared to PBS (P=0.0007 and 0.0002 respectively; FIG. 15d-e). In contrast, the control mixture did not reduce the fractions of these two populations of PD-1+ lymphocytes; the control mixture slightly increased the PD-1⁺ CD4 T fraction. These fraction values also revealed that PD-1⁺ CD4 T cells are the major PD-1⁺ cells in the pancreases of the PBS treated mice; they account for approximately 75% of the PD-1⁺ cells. There was no detectable population of PD-1⁺ Tregs or PD-1⁺ B cells in the pancreases (FIG. 17b-c). The αPD-1-ABD-PE treatment also reduced total CD4 and CD8 cells but not B cells in the pancreases, as compared to PBS (P=0.0002, 0.0008, and 0.119, respectively; FIG. 15f-h). The mixture control, as expected, did not affect these cells. Thus, αPD-1-ABD-PE reduced the fractions of total PD-1⁺ cells, PD-1⁺ CD4 T cells, PD-1⁺ CD8 T cells, CD4 T cells, and CD8 T cells in the pancreas of treated mice, which might contribute to its marked delaying effect on T1D onset. Among these population, PD-1⁺ CD4 T cells are likely a decisive factor given that they represent a major fraction of the PD-1⁺ cells (75%) and their numbers were reduced by almost 3 times after the αPD-1-ABD-PE treatment. Next, PD-1⁺ T cells and the PD-1⁺ B cells in blood, spleens, and lymph nodes of these treated mice were also examined. However, these cells did not form distinguishable populations likely due to their scarcity (FIG. 17d-i). In other words, the baseline numbers of PD-1⁺ cells in blood and peripheral lymphatic organs are low. Because of this result, it is expected that the total T and B cell numbers in these organs are not altered by αPD-1-ABD-PE treatments.

In addition to the pancreatic infiltration of lymphocytes, there was other data suggesting that αPD-1-ABD-PE selectively depletes autoreactive PD-1⁺ cells in NOD mice (FIG. 15i). Normally, αPD-1 accelerates T1D progression in NOD mice because it allows autoreactive PD-1⁺ cells to proliferate (Salama, A. D. et al. Critical role of the programmed death-1 (PD-1) pathway in regulation of experimental autoimmune encephalomyelitis. J Exp Med 198, 71-78 (2003); Godwin, J. L. et al. Nivolumab-induced autoimmune diabetes mellitus presenting as diabetic ketoacidosis in a patient with metastatic lung cancer. Journal for immunotherapy of cancer 5, 40 (2017); Ansari, M. J. et al. The programmed death-1 (PD-1) pathway regulates autoimmune diabetes in nonobese diabetic (NOD) mice. J Exp Med 198, 63-69 (2003); Hughes, J. et al. Precipitation of autoimmune diabetes with anti-PD-1 immunotherapy. Diabetes Care 38, e55-57 (2015); and Zhao, P. et al. An Anti-Programmed Death-1 Antibody (αPD-1) Fusion Protein That Self-Assembles into a Multivalent and Functional αPD-1 Nanoparticle. Molecular pharmaceutics 14, 1494-1500 (2017)). In this study, αPD-1 accelerated the onset of T1D in NOD mice that were pretreated with PBS and the mixture control: the median T1D-free survivals were 15 and 13 days, respectively (FIG. 15i). In contrast, T1D exacerbating effect of αPD-1 diminished in αPD-1-ABD-PE pre-treated NOD mice: the mice did not show hyperglycemia even at the end of the experiment (26 days after the treatment). Because autoreactive PD-1⁺ cells are primary effector cells that cause T1D and execute exacerbating effects of αPD-1, the diminished exacerbation effect of αPD-1 in the αPD-1-ABD-PE pre-treated mice suggests a reduction of autoreactive PD-1+ cells in these mice.

αPD-1-ABD-PE Treated Mice Recover from EAE.

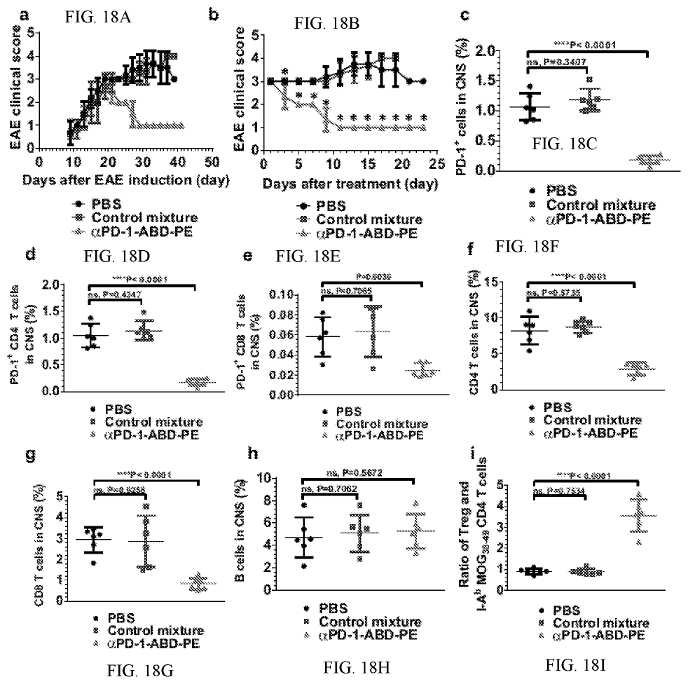

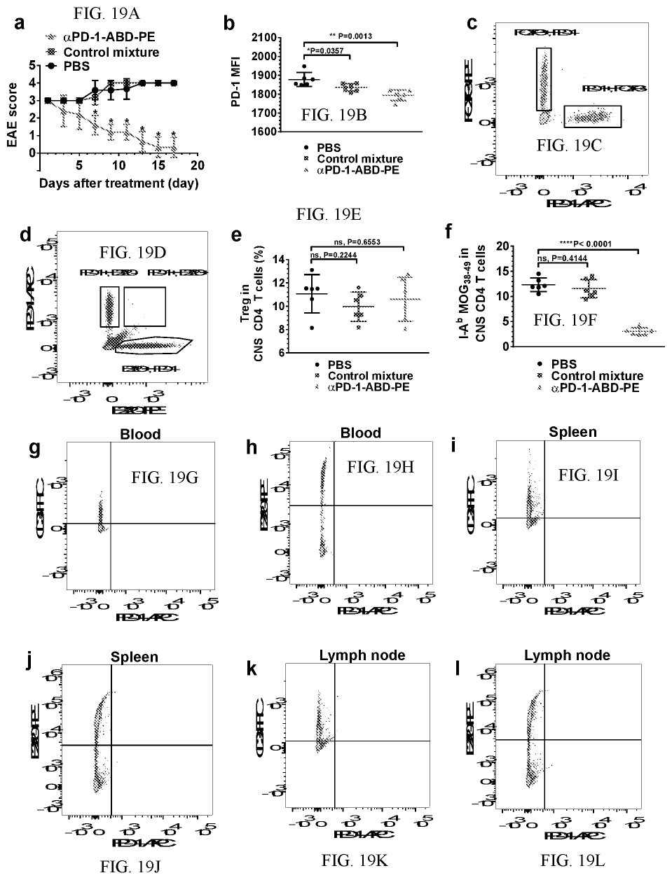

C57BL/6 mice immunized with MOG₃₅₋₅₅ in adjuvant develop a monophasic clinical disease where mice rarely recover (Wang, Y. et al. Neuropilin-1 modulates interferon-gamma-stimulated signaling in brain microvascular endothelial cells. J Cell Sci 129, 3911-3921 (2016) and Rangachari, M. & Kuchroo, V. K. Using EAE to better understand principles of immune function and autoimmune pathology. J Autoimmun 45, 31-39 (2013)). To test whether αPD-1-ABD-PE modulates EAE, C57BL/6 mice with EAE were treated with either, 1) αPD-1-ABD-PE, 2) PBS, or 3) a mixture of αPD-1 and ABD-PE. Before the treatment, the mice had paralyzed hind limbs, a sign of severe EAE (clinical score 3.0). The αPD-1-ABD-PE-treated mice recovered from the paralytic diseases after a single dose (FIG. 18a-b) and had an EAE clinical score of 1.0 at the end of the study. In another replicate study, two of five αPD-1-ABD-PE-treated mice had no clinical sign of EAE (fully recovered) at the end of this study (clinical score 0, FIG. 19a). The other three mice in this replicate study had clinical scores of 1.0. In contrast, none of the paralyzed mice that received PBS or the mixture control treatment recovered. Instead, their EAE steadily worsened. The EAE scores of the mice in these two groups reached 4.00 by the end of this study. Indeed, from day 3 post treatment, the mean clinical score of the αPD-1-ABD-PE treated group was consistently and significantly lower than the scores of the PBS- and the mixture control-treated groups (P<0.0001). It is noteworthy that mice in the three groups developed EAE with the same kinetics before the various treatments (FIG. 18a): there was no significant difference in mean EAE scores among the three groups until day 19 after EAE induction (P>0.05 for the monitored time points). At that time, some mice had received treatments, and the treatment had started to alter their EAE progression. The same EAE development kinetics among the three groups before the treatments, conversely, underscores the power of αPD-1-ABD-PE to interfere with and reverse the disease progression.

It was next investigated whether αPD-1-ABD-PE treatment reduced the infiltrations of PD-1⁺ cells and lymphocytes into CNS of the mice with EAE. The presence of these cells especially CD4 cells in the CNS has been linked to EAE progression (Zhu, B. et al. Differential role of programmed death-ligand 1 [corrected] and programmed death-ligand 2 [corrected] in regulating the susceptibility and chronic progression of experimental autoimmune encephalomyelitis. J Immunol 176, 3480-3489 (2006); Salama, A. D. et al. Critical role of the programmed death-1 (PD-1) pathway in regulation of experimental autoimmune encephalomyelitis. J Exp Med 198, 71-78 (2003); and Schreiner, B., Bailey, S. L., Shin, T., Chen, L. & Miller, S. D. PD-1 ligands expressed on myeloid-derived APC in the CNS regulate T-cell responses in EAE. Eur J Immunol 38, 2706-2717 (2008)). αPD-1-ABD-PE, even at a single dose, reduced the fraction of PD-1⁺ cells in the CNS by more than 5 times as compared to PBS (FIG. 18c; 0.19±0.03% versus 1.07±0.09%, P<0.0001). In contrast, the control mixture of αPD-1 and ABD-PE did not reduce the fraction of PD-1⁺ cells in the CNS (1.19±0.07%, P=0.341). In regard of the PD-1 expression (MFI) of these PD-1⁺ cells, the PD-1⁺ cells collected from the αPD-1-ABD-PE treated mice had significantly lower MFI (1793.83±11.55, P=0.0013) than the cells from the PBS treated mice (1877.83±15.2, FIG. 19b). Interestingly, the cells collected from the control mixture-treated mice had slightly lower MFI (1836.00±8.14) as well. Further, αPD-1-ABD-PE significantly reduced the fractions of PD-1⁺ CD4 T, and PD-1⁺ CD8 T cells in the CNS as compared to PBS (P<0.0001 and P=0.003 respectively; FIG. 18d-e). In contrast, the control mixture did not reduce the fractions of these PD-1⁺ lymphocytes in the CNS. PD-1⁺ CD4 T cells account for the vast majority of PD-1⁺ cells in the CNS of the mice with EAE (98.1% in the PBS treated mice). There was no detectable population of PD-1⁺ Tregs or PD-1⁺ B cells in the CNS (FIG. 19c-d). The αPD-1-ABD-PE treatment also reduced total CD4 T and CD8 T cells but not B cells in the CNS, as compared to PBS (P<0.0001, P=0.0001, and P=0.567 respectively; FIG. 18f-h). The mixture control, as expected, did not affect these cells. Lastly, the ratios between Tregs and MOG peptide-specific CD4 T cells (MOG₃₈₋₄₉: GWYRPPFSRVVH) in the CNS was compared among the three treatments. This MOG-specific clone is the main autoreactive T effector cell clone in this EAE model (Massilamany, C., Upadhyaya, B., Gangaplara, A., Kuszynski, C. & Reddy, J. Detection of autoreactive CD4 T cells using major histocompatibility complex class II dextramers. BMC immunology 12, 40 (2011)). It was found that the αPD-1-ABD-PE treatment boosted the ratio by approximately 4 times, as compared to PBS (P<0.0001, FIG. 18i). The increased ratio was due to the fact that the Treg fraction in the CNS was not changed after the αPD-1-ABD-PE treatment (FIG. 19e) but the MOG-specific CD4 T cells decreased (FIG. 190. The mixture control did not significantly alter the ratio (FIG. 18i). Taken together, these results suggest that the αPD-1-ABD-PE treatment diminished the infiltrations of PD-1⁺ cells and T cells into the CNS of treated mice and the treatment increase the Treg-to-effector cell ratio, a sign favoring immune tolerance.

PD-1⁺ T cells and the PD-1⁺ B cells in blood, spleens, and lymph nodes of treated mice was also examined, same as what was conducted in NOD mice. However, these populations were not distinguished from one another (FIG. 19g-1).

αPD-1-ABD-PE does not Compromise Normal Adaptive Immune Responses.

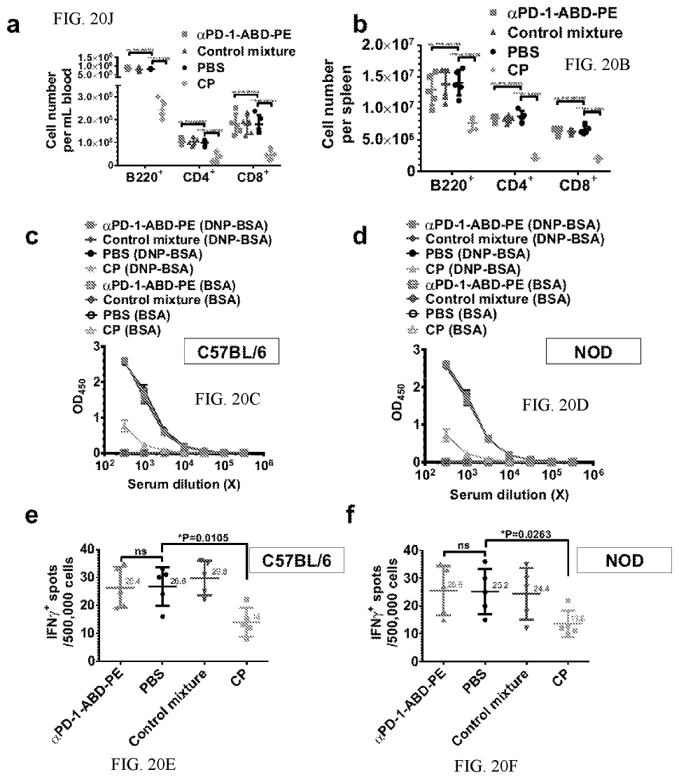

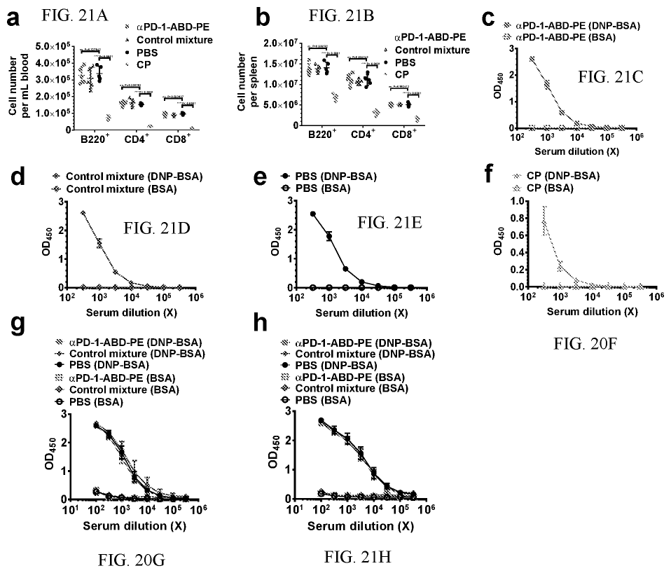

The αPD-1-ABD-PE treatment did not cause lymphopenia that is often associated with therapeutics for autoimmune diseases (Wingerchuk, D. M. & Carter, J. L. Multiple sclerosis: current and emerging disease-modifying therapies and treatment strategies. Mayo Clin Proc 89, 225-240 (2014); Elsegeiny, W., Eddens, T., Chen, K. & Kolls, J. K. Anti-CD20 antibody therapy and susceptibility to Pneumocystis pneumonia. Infection and immunity 83, 2043-2052 (2015); Genzyme (Cambridge, Mass.; 2014); Torkildsen, Ø., Myhr, K. M. & Bø, L. Disease-modifying treatments for multiple sclerosis—a review of approved medications. European Journal of Neurology 23, 18-27 (2016); McNamara, C., Sugrue, G., Murray, B. & MacMahon, P. J. Current and Emerging Therapies in Multiple Sclerosis: Implications for the Radiologist, Part 2-Surveillance for Treatment Complications and Disease Progression. AJNR Am J Neuroradiol (2017); and Turner, M. J. et al. Immune status following alemtuzumab treatment in human CD52 transgenic mice. J Neuroimmunol 261, 29-36 (2013)). B220+, CD4+ and CD8+ lymphocytes in blood (FIG. 20a) and spleens (FIG. 20b) of C57BL/6 mice were compared after mice received one dose of 1) αPD-1-ABD-PE, 2) PBS, 3) a mixture of αPD-1 and ABD-PE, or 4) cyclophosphamide (CP). CP is a non-specific immunosuppressant used in these experiments as a positive control for immune suppression. αPD-1-ABD-PE did not reduce the numbers of these three lymphocyte sub-populations as compared to the PBS treatment (FIG. 20a-b). Similarly, the mixture control did not affect the proportions of these lymphocyte sub-populations. However, CP reduced the numbers of B220+, CD4+ and CD8+ lymphocytes in blood by an average of 70.1%, 69.5%, and 75.6%, respectively (P<0.0001, P=0.0002, and P=0.0002, respectively; FIG. 20a); CP reduced the numbers of B220+, CD4+ and CD8+ lymphocytes in spleens by 46.0%, 76.0%, and 70.1%, respectively (P=0.0002, P<0.0001 and P<0.0001, respectively; FIG. 20b). These findings were reproducible in NOD mice (FIG. 21a-b).

The αPD-1-ABD-PE treatment did not affect antibody responses in treated mice. First C57BL/6 and NOD mice were treated with one dose of 1) αPD-1-ABD-PE, 2) PBS, 3) a mixture of αPD-1 and ABD-PE, or 4) CP. Two days later, these mice were immunized with DNP-Ficoll, a T cell-independent antigen (Sharon, R., McMaster, P. R., Kask, A. M., Owens, J. D. & Paul, W. E. DNP-Lys-ficoll: a T-independent antigen which elicits both IgM and IgG anti-DNP antibody-secreting cells. J Immunol 114, 1585-1589 (1975)). It was found that both the αPD-1-ABD-PE-treated mice and the mixture control-treated mice developed the same level of anti-DNP responses as the PBS-treated mice (FIG. 20c-d, FIG. 21c-f). However, CP-treated mice developed weaker anti-DNP responses. These findings were reproduced in both C57BL/6 (FIG. 20c) and NOD (FIG. 20d) mice. It is noteworthy that the same conclusion was reached in mice that were treated with five-doses of αPD-1-ABD-PE and its controls (FIG. 21g-h).

The αPD-1-ABD-PE treatment also did not affect cytotoxic T lymphocyte (CTL) responses. First C57BL/6 and NOD mice were treated with one dose of 1) αPD-1-ABD-PE, 2) PBS, or 3) a mixture of αPD-1 and ABD-PE, or 4) CP. Two days after the dosing, treated NOD mice were immunized with TYQRTRALV (SEQ ID NO: 16), a CTL epitope vaccine that matched with the MHC class I background of NOD mice; whereas, C57BL/6-treated mice were immunized with SIINFEKL (SEQ ID NO: 17), a CTL vaccine that was derived from ovalbumin (residues 257-264) and matched with the MHC class I background of C57BL/6 mice. It was observed that the αPD-1-ABD-PE-treated mice, the mixture control-treated mice and the PBS-treated mice developed the same degree of CTL responses (FIG. 20e-f). In contrast, the CP-treated mice developed weaker CTL responses as compared to the PBS treated mice (p=0.011 and 0.026 for C57BL/6 and NOD mice, respectively). These findings were noted in both C57BL/6 (FIG. 20e) and NOD (FIG. 200 and mice.

These data, together, showed that αPD-1-ABD-PE treatment did not significantly alter the ability of the treated mice to mount antibody and/or CTL responses.

Discussion.

According to results of this study, αPD-1-ABD-PE has specific toxicity to PD-1⁺ B and T cells; PD-1⁺ cell depletion, enabled by αPD-1-ABD-PE, ameliorates autoimmunity in multiple disease models that are different in pathogenesis; and application of PD-1⁺ cell depletion does not cause adaptive immune deficiency.

PD-1⁺ cell depletion was strikingly effective in several autoimmune disease models. In a chronic EAE model, even a single dose of αPD-1-ABD-PE fully restored mobility in mice that were paralyzed by EAE, which is a rarely achieved therapeutic outcome. In addition, the single dose of αPD-1-ABD-PE significantly reduced PD-1⁺ cell, T lymphocytes, and autoreactive lymphocytes in CNS of the treated mice. Current therapies for MS are termed disease-modifying therapies because they are limited to delaying disease progression and do not reverse (or cure) the disease (Wingerchuk, D. M. & Carter, J. L. Multiple sclerosis: current and emerging disease-modifying therapies and treatment strategies. Mayo Clin Proc 89, 225-240 (2014); and Torkildsen, Ø., Myhr, K. M. & Bø, L. Disease-modifying treatments for multiple sclerosis—a review of approved medications. European Journal of Neurology 23, 18-27 (2016)). The noted efficacy of PD-1⁺ cell depletion may render the depletion able to fill a significant clinical gap in MS treatment. Regarding T1D, PD-1⁺ cell depletion markedly delayed T1D onset in a spontaneous model as well as in two accelerated T1D models (CP treatment and αPD-1 IgG treatment). Under the conditions studied, T1D eventually developed in the spontaneous and the CP-accelerated models after significant delays. Nevertheless, it is possible that the efficacy of PD-1⁺ cell depletion will be further boosted by regimens that include αPD-1-ABD-PE. The improved efficacy may allow the depletion to completely prevent T1D. This possibility is supported by the observed broad impact of a single dose of αPD-1-ABD-PE on immune cells in the pancreas; the dose decreased the fractions of PD-1⁺ CD4 T cells, PD-1⁺ CD8 T cells, as well as total CD4 and CD8 T cells, a global attenuation of the inflammation in the pancreas. PD-1⁺ cell depletion may also be helpful to reverse T1D in combination with α-cell compensation therapy (Atkinson, M. A., Eisenbarth, G. S. & Michels, A. W. Type 1 diabetes. Lancet 383, 69-82 (2014); and van Belle, T. L., Coppieters, K. T. & von Herrath, M. G. Type 1 diabetes: etiology, immunology, and therapeutic strategies. Physiol Rev 91, 79-118 (2011)). It is noteworthy that PD-1⁺ cell depletion delayed the onset of CP-induced T1D. First, the observation implies that PD-1⁺ cells may be the primary effector cells in autoimmunity driven by Treg deficiency since CP causes Treg deficiency (Brode, S., Raine, T., Zaccone, P. & Cooke, A. Cyclophosphamide-induced type-1 diabetes in the NOD mouse is associated with a reduction of CD4+CD25+Foxp3+ regulatory T cells. J Immunol 177, 6603-6612 (2006)). Second, this observation suggests that the depletion may be able to resolve the autoimmunity that does not originate from the abnormality of the PD-1 checkpoint. Together, PD-1⁺ cell depletion is efficacious in EAE and T1D, the two T cell-driven autoimmune disease models; PD-1⁺ CD4 T cells appeared to be the major pathogenic cell population in the two models and responded well to PD-1⁺ cell depletion.

PD-1⁺ cell depletion will likely be effective to B-cell mediated autoimmune diseases. First, these autoimmune diseases like SLE depend on B cells as well as CD4 T cells such as Tfh and extrafollicular Th. These CD4 T cells facilitate the formation of a germinal center, the maturation of B cells, and antibody production (Zhang, Q. & Vignali, D. A. Co-stimulatory and Co-inhibitory Pathways in Autoimmunity. Immunity 44, 1034-1051 (2016)). Meanwhile, these results show that PD-1⁺ cell depletion diminished both PD-1+CD4 T cells and total CD4 T cells (FIGS. 15 and 18). Further, these results confirmed that αPD-1-ABD-PE is able to specifically bind to, penetrate, and eliminate PD-1⁺ primary B cells. Last, PD-1 knockout mice have elevated IgG production and eventually assume lupus-like symptoms (Zhang, Q. & Vignali, D. A. Co-stimulatory and Co-inhibitory Pathways in Autoimmunity. Immunity 44, 1034-1051 (2016)). One interpretation for this observation is that activated autoreactive cells in the mice escape the PD-1 immune checkpoint and cause the lupus-like symptoms since these cells do not express PD-1. In contrast, activated autoreactive cells are PD-1-positive in wildtype mice and hence inhibited by the checkpoint. In mice with SLE, the checkpoint fails to suppress activated autoreactive cells. However, it is possible to utilize PD-1 as a biomarker to identify and deplete the cells and assuage autoimmune destruction in the mice with SLE. Taken together, PD-1⁺ cell depletion impacts both PD-1⁺ B and CD4 T cells and likely alleviates B-cell mediated autoimmune diseases.

PD-1⁺ cell depletion preserves normal adaptive immunity, which sharply contrasts from the generalized immune deficiency caused by currently available immune suppressants (Wingerchuk, D. M. & Carter, J. L. Multiple sclerosis: current and emerging disease-modifying therapies and treatment strategies. Mayo Clin Proc 89, 225-240 (2014); Elsegeiny, W., Eddens, T., Chen, K. & Kolls, J. K. Anti-CD20 antibody therapy and susceptibility to Pneumocystis pneumonia. Infection and immunity 83, 2043-2052 (2015); Genzyme (Cambridge, Mass.; 2014); Torkildsen, Ø., Myhr, K. M. & Bø, L. Disease-modifying treatments for multiple sclerosis—a review of approved medications. European Journal of Neurology 23, 18-27 (2016); McNamara, C., Sugrue, G., Murray, B. & MacMahon, P. J. Current and Emerging Therapies in Multiple Sclerosis: Implications for the Radiologist, Part 2-Surveillance for Treatment Complications and Disease Progression. AJNR Am J Neuroradiol (2017); and Turner, M. J. et al. Immune status following alemtuzumab treatment in human CD52 transgenic mice. J Neuroimmunol 261, 29-36 (2013)). It is acknowledged that PD-1⁺ cell depletion could diminish non-autoreactive PD-1⁺ lymphocytes including PD-1⁺ effector cells and Tregs (Sharpe, A. H. & Pauken, K. E. The diverse functions of the PD1 inhibitory pathway. Nat Rev Immunol 18, 153-167 (2018)). However, this may represent a theoretical concern. First, PD-1⁺ effector cells can be replenished from naïve lymphocytes upon immune stimulation as lymphocyte repertoires are not impaired by PD-1⁺ cell depletion. Second, PD-1⁺ cells are scarce and hardly detectable in blood and peripheral lymphatic organs in mice with T1D and EAE, rather they are relatively concentrated in inflamed organs (FIGS. 15, 18, 17, and 19). This intrinsic distribution of PD-1⁺ cells helps to alleviate the negative impact of PD-1⁺ cell depletion on non-autoreactive lymphocytes. Last, the finding that PD-1⁺ cell depletion preserves adaptive immunity argues against the theoretical concern; mice that experienced the depletion were able to mount normal immune responses as soon as two days after the depletion. There is additional rational supporting the depletion of PD-1⁺ cells in autoimmune diseases including non-autoreactive PD-1⁺ cells. In the context of autoimmune diseases, the immune system is, overall, tilted toward autoimmunity rather than immune tolerance or anergy (Davidson, A. & D'iamond, B. in The Autoimmune Diseases, Edn. Fifth Edition. (ed. N. R. M. Rose, I. R.) 19-37 (Elsevier Inc, San Diego, Calif., USA; 2014)). Autoreactive, PD-1⁺ lymphocytes outweigh their non-autoreactive counterparts including PD-1⁺ Tregs (Sharpe, A. H. & Pauken, K. E. The diverse functions of the PD1 inhibitory pathway. Nat Rev Immunol 18, 153-167 (2018)) in terms of impact to the well-being of patients. Thus, it is arguably beneficial to purge PD-1⁺ cells, reprogram the immune system, and restore immune homeostasis. PD-1⁺ cell depletion, indeed, resembles systemic γ-irradiation or lymphocyte depletion that have both been used to reprogram the immune system (Kasagi, S. et al. In vivo-generated antigen-specific regulatory T cells treat autoimmunity without compromising antibacterial immune response. Sci Transl Med 6, 241ra278 (2014)). Yet, PD-1⁺ cell depletion is a much more focused approach than these reported approaches. One observation that supports this idea of purging PD-1⁺ cells is that the αPD-1-ABD-PE treatment, while reducing PD-1⁺ CD4 T cells, PD-1⁺ CD8 T cells, and autoreactive T cells in the pancreas of NOD mice as well as the CNS of the mice with EAE, did not diminish Tregs in these organs. Indeed, the depletion increased the ratios between Treg and autoreactive cells in the CNS, which favors the treatment of autoimmune diseases.

Targeted depletion of PD-1⁺ cells, enabled by αPD-1-ABD-PE, has shown three appealing features as a new therapeutic option for autoimmune diseases. First, the depletion has a straightforward and robust working mechanism. It relies on a simple cytotoxic mechanism to kill PD-1⁺ cells; yet, a single administration of depletion can drastically suppress autoimmunity completely such as enabling paralyzed mice to regain normal gait. Second, the depletion is capable to suppress both the T and B cell mediated autoimmunity in one autoimmune disease. It also has potential to treat primarily T cell- or B cell-driven autoimmune diseases. Last, the depletion does not cause long-term immune deficiency. Although the depletion can deplete non-autoreactive PD-1⁺ cells and may affect the healthy immunity at the moment of the action, the immunity appears to recover quickly because of the preservation of naive cells. Indeed, as soon as two days after the depletion, the αPD-1-ABD-PE treated mice were able to mount the same strength of adaptive responses as the PBS treated mice. Thus, the depletion did not impact healthy immunity for the long-term. In summary, targeted depletion of PD-1⁺ cells is an effective and broadly applicable approach to treat autoimmune diseases without jeopardizing healthy immunity.

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

1. A fusion protein comprising a targeting moiety, a plasma protein binding domain, and a toxin or biological variant thereof.

2. The fusion protein of claim 1, wherein the targeting moiety is a single chain variable fragment (scFv) of an anti-PD-1 antibody or an anti-CTLA antibody, the plasma protein binding domain is an albumin-binding protein domain and the toxin is a Pseudomonas exotoxin or a biological variant thereof.

3. The fusion protein of claim 2, wherein the fusion protein comprises, from the N-terminus to the C-terminus, a single chain variable fragment (scFv) of an anti-PD-1 antibody, a peptide linker, an albumin-binding protein domain, a second peptide linker, and a Pseudomonas exotoxin or biological variant thereof.

4. The fusion protein of claim 3, wherein the Pseudomonas exotoxin or biological variant thereof comprises SEQ ID NO: 5.

5. (canceled)

6. (canceled)

7. (canceled)

8. (canceled)

9. (canceled)

10. (canceled)

11. (canceled)

12. (canceled)

13. (canceled)

14. The fusion protein of claim 2, wherein the anti-PD-1 antibody is nivolumab, pembrolizumab, pidilizumab, BMS-936559, MEDI0680, clone J116 or a biologically active variant thereof.

15. (canceled)

16. (canceled)

17. (canceled)

18. (canceled)

19. (canceled)

20. (canceled)

21. (canceled)

22. (canceled)

23. The fusion protein of claim 2, wherein the targeting moiety is an anti-CTLA antibody, wherein the anti-CTLA-antibody is ipilimumab, tremelimumab, UC10-4F10 clone or a biologically active variant thereof.

24. (canceled)

25. (canceled)

26. (canceled)

27. (canceled)

28. (canceled)

29. (canceled)

30. (canceled)

31. The fusion protein of claim 2, wherein the albumin-binding protein domain comprises SEQ ID NO: 6.

32. (canceled)

33. (canceled)

34. (canceled)

35. (canceled)

36. (canceled)

37. A pharmaceutical composition comprising the fusion protein of claim 1 and a pharmaceutically acceptable carrier.

38. (canceled)

39. (canceled)

40. A method of treating a subject with an autoimmune disease, the method comprising:
a. Identifying a subject in need of treatment; and
b. Administering to the subject a therapeutically effective amount of the pharmaceutical composition of claim 37.

41. (canceled)

42. The method of claim 40, wherein the autoimmune disease is non-Hodgkin's lymphoma, rheumatoid arthritis, chronic lymphocytic leukemia, multiple sclerosis, systemic lupus erythematosus, autoimmune hemolytic anemia, pure red cell aplasia, idiopathic thrombocytopenic purpura, Evans syndrome, vasculitis, bullous skin disorders, type 1 diabetes mellitus, Sjögren's syndrome, Devic's disease, or Graves' disease ophthalmopathy.

43. (canceled)

44. (canceled)

45. (canceled)

46. A method of treating or ameliorating one or more symptoms of Type I diabetes in a subject, the method comprising:
a. Identifying a subject in need of treatment; and
b. Administering to the subject a therapeutically effective amount of the pharmaceutical composition of claim 37.

47. (canceled)

48. A method of inducing apoptosis, the method comprising: contacting a cell with a composition comprising a fusion protein, wherein the fusion protein comprises a single chain variable fragment (scFv) of an anti-PD-1-antibody or an anti-CTLA-4-antibody, a plasma protein binding domain and a toxin or a biological variant thereof; wherein the contacting of the cells with the composition induces apoptosis.

49. The method of claim 48, wherein the single chain variable fragment (scFv) of the anti-PD-1-antibody is an scFV of nivolumab, pembrolizumab, pidilizumab or BMS-936559, MEDI0680, clone J116, or a biologically active variant thereof.

50. The method of claim 48, wherein the plasma protein binding domain is an albumin-binding protein domain.

51. The method of claim 48, wherein the toxin is Pseudomonas exotoxin or a biologically active variant thereof.

52. (canceled)

53. (canceled)

54. (canceled)

55. The method of claim 48, wherein the cell is in a subject, wherein the subject has Type I diabetes, multiple sclerosis, arthritis or is undergoing an organ transplant.

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. A method of preventing or halting cell death of one or more islet cells, the method comprising: contacting one or more lymphocytes with a composition comprising a fusion protein, wherein the fusion protein comprises a single chain variable fragment (scFv) of an anti-PD-1 antibody, a plasma protein binding domain and a toxin or biological variant thereof, wherein the contacting of the one or more lymphoctyes with the composition prevents or halts cell death.

69. The method of claim 68, wherein the one or more lymphocytes are T cells or B cells.

70. The method of claim 69, wherein the T cells or B cells are not depleted.

71. (canceled)

72. (canceled)

73. (canceled)

74. (canceled)

75. (canceled)

76. (canceled)


SEQUENCE LISTING (27 sequences)

SEQ ID NO: 1 - protein, 15 aa, Artificial Sequence
  Synthetic construct
GGGGSGGGGSGGGGS

SEQ ID NO: 2 - protein, 5 aa, Artificial Sequence
  Synthetic construct
GGGGS

SEQ ID NO: 3 - protein, 10 aa, Artificial Sequence
  Synthetic construct
GGGGSGGGGS

SEQ ID NO: 4 - protein, 20 aa, Artificial Sequence
  Synthetic construct
GGGGSGGGGSGGGGSGGGGS

SEQ ID NO: 5 - protein, 230 aa, Artificial Sequence
  Synthetic construct
RHRQPRGWEQLPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 6 - protein, 46 aa, Artificial Sequence
  Synthetic construct
LAEAKVLANRELDKYGVSDFYKRLINKAKTVEGVEALKLHILAALP

SEQ ID NO: 7 - protein, 8 aa, Artificial Sequence
  Synthetic construct
SIINFEKL

SEQ ID NO: 8 - protein, 9 aa, Artificial Sequence
  Synthetic construct
SVYDFFVWL

SEQ ID NO: 9 - protein, 21 aa, Artificial Sequence
  Synthetic construct
MEVGWYRSPFSRVVHLYRNGK

SEQ ID NO: 10 - protein, 13 aa, Artificial Sequence
  Synthetic construct
HSLGKWLGHPDKF

SEQ ID NO: 11 - protein, 12 aa, Artificial Sequence
  Synthetic construct
VHFFKNIVPRTP

SEQ ID NO: 12 - protein, 13 aa, Artificial Sequence
  Synthetic construct
TGILDSIGRFFSG

SEQ ID NO: 13 - protein, 46 aa, Artificial Sequence
  Synthetic construct
LAEAKVLANRELDKYGVSDYYKNLINNAKTVEGVKALIDEILAALP

SEQ ID NO: 14 - protein, 46 aa, Artificial Sequence
  Synthetic construct
LAEAKVLANRELDKYGVSDYYKNIINRAKTVEGVRALKLHILAALP

SEQ ID NO: 15 - protein, 46 aa, Artificial Sequence
  Synthetic construct
LAEAKVLANRELDKYGVSDYYKNLINKAKTVEGVEALTLHILAALP

SEQ ID NO: 16 - protein, 9 aa, Artificial Sequence
  Synthetic construct
TYQRTRALV

SEQ ID NO: 17 - DNA, 30 bp, Artificial Sequence
  Synthetic construct
ccttgacacacggcgcaatgacagtggcat

SEQ ID NO: 18 - DNA, 25 bp, Artificial Sequence
  Synthetic construct
caccggacacacggcgcaatgacag

SEQ ID NO: 19 - DNA, 25 bp, Artificial Sequence
  Synthetic construct
aaacctgtcattgcgccgtgtgtcc

SEQ ID NO: 20 - protein, 120 aa, Artificial Sequence
  Synthetic construct
EVQLQESGPGLVKPSQSLSLTCSVTGYSITSSYRWNWIRKFPGNCLEWMGYINSAGISNYNPSLKRRISITRDTSKNQFFLQVNSVTTEDAATYYCARSDNMGTTPFTYWGQGTLVTVSS

SEQ ID NO: 21 - protein, 111 aa, Artificial Sequence
  Synthetic construct
DIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGCGTKLELK

SEQ ID NO: 22 - protein, 6 aa, Artificial Sequence
  Synthetic construct
SSYRWN

SEQ ID NO: 23 - protein, 16 aa, Artificial Sequence
  Synthetic construct
YINSAGISNYNPSLKR

SEQ ID NO: 24 - protein, 11 aa, Artificial Sequence
  Synthetic construct
SDNMGTTPFTY

SEQ ID NO: 25 - protein, 16 aa, Artificial Sequence
  Synthetic construct
RSSKSLLYSDGKTYLN

SEQ ID NO: 26 - protein, 7 aa, Artificial Sequence
  Synthetic construct
WMSTRAS

SEQ ID NO: 27 - protein, 8 aa, Artificial Sequence
  Synthetic construct
QQGLEFPT